SEQUENCE LISTING (56 sequences)

SEQ ID NO: 1 - DNA, 30 bp, Artificial
  PCR Primer
gaggtcgacgcgatcgataagcttgatatc

SEQ ID NO: 2 - DNA, 28 bp, Artificial
  PCR Primer
tagaactagtcgatcgcccgggctgcag

SEQ ID NO: 3 - DNA, 72 bp, Artificial
  Synthetic oligonucleotide
gattccaattgctgagttggaatccattttcagcgggagccacctgatgaagcttgatcgggtggctctctt

SEQ ID NO: 4 - DNA, 78 bp, Artificial
  Synthetic oligonucleotide
gatcaaaaaacaggtggctcccgctgaatttggattccaactcagcgagagccacccgatcaagcttcatggaatccg

SEQ ID NO: 5 - DNA, 30 bp, Artificial
  PCR Primer
ccagaagagctcgcaacgtggcatctgcta

SEQ ID NO: 6 - DNA, 29 bp, Artificial
  PCR Primer
gtttggagagctcctgggcacaatgaggc

SEQ ID NO: 7 - DNA, 26 bp, Artificial
  PCR Primer
acaacggtaccgaacttaagctgcag

SEQ ID NO: 8 - DNA, 27 bp, Artificial
  PCR Primer
ccgaaaggtaccacctggatccttatc

SEQ ID NO: 9 - DNA, 29 bp, Artificial
  Mutated target recognition site of firefly luciferase
accaggttgcccctgctgagttggaatcg

SEQ ID NO: 10 - DNA, 27 bp, Artificial
  Synthetic oligonucleotide
accaggttgcccctgctgagttggaat

SEQ ID NO: 11 - DNA, 29 bp, Artificial
  Synthetic oligonucleotide
cgattccaactcagcaggggcaacctggt

SEQ ID NO: 12 - DNA, 64 bp, Artificial
  Synthetic oligonucleotide
gatccccctaacactgggttatacaattcaagagattgtataacccagtgttagtttttggaaa

SEQ ID NO: 13 - DNA, 64 bp, Artificial
  Synthetic oligonucleotide
agcttttccaaaaactaacactgggttatacaatctcttgaattgtataacccagtgttagggg

SEQ ID NO: 14 - DNA, 52 bp, Artificial
  Synthetic oligonucleotide
ggcggctttgccaagtgcttctcgagaagcacttggcaaagccgcccttttt

SEQ ID NO: 15 - DNA, 58 bp, Artificial
  Synthetic oligonucleotide
gatcaaaaagggcggctttgccaagtgcttctcgagaagcacttggcaaagccgcccg

SEQ ID NO: 16 - DNA, 52 bp, Artificial
  Synthetic oligonucleotide
gccgcctccctcatccagaactcgagttctggatgagggaggcggccttttt

SEQ ID NO: 17 - DNA, 58 bp, Artificial
  Synthetic oligonucleotide
gatcaaaaaggccgcctccctcatccagaactcgagttctggatgagggaggcggccg

SEQ ID NO: 18 - DNA, 56 bp, Artificial
  Synthetic oligonucleotide
atgaagaagatcaccctccttactcgagtaaggagggtgatcttcttcatcttttt

SEQ ID NO: 19 - DNA, 62 bp, Artificial
  Synthetic oligonucleotide
gatcaaaaagatgaagaagatcaccctccttactcgagtaaggagggtgatcttcttcatcg

SEQ ID NO: 20 - DNA, 56 bp, Artificial
  Synthetic oligonucleotide
gaagctttcctcgagatccacttcctgtcatggatctcgaggaaagcttccttttt

SEQ ID NO: 21 - DNA, 62 bp, Artificial
  Synthetic oligonucleotide
gatcaaaaaggaagctttcctcgagatccatgacaggaagtggatctcgaggaaagcttccg

SEQ ID NO: 22 - DNA, 56 bp, Artificial
  Synthetic oligonucleotide
gcatcgagcagcacatggatcttcctgtcaatccatgtgctgctcgatgccttttt

SEQ ID NO: 23 - DNA, 62 bp, Artificial
  Synthetic oligonucleotide
gatcaaaaaggcatcgagcagcacatggattgacaggaagatccatgtgctgctcgatgccg

SEQ ID NO: 24 - DNA, 56 bp, Artificial
  Synthetic oligonucleotide
ctcgccaggagaagcggtttcttcctgtcaaaaccgcttctcctggcgagcttttt

SEQ ID NO: 25 - DNA, 62 bp, Artificial
  Synthetic oligonucleotide
gatcaaaaagctcgccaggagaagcggttttgacaggaagaaaccgcttctcctggcgagcg

SEQ ID NO: 26 - DNA, 20 bp, Artificial
  Synthetic oligonucleotide
tcaggactagtggaatgtac

SEQ ID NO: 27 - DNA, 20 bp, Artificial
  Synthetic oligonucleotide
attccactagtcctgagtac

SEQ ID NO: 28 - DNA, 56 bp, Artificial
  Synthetic oligonucleotide
ctgcacctgacgcccttcaccttcctgtcagtgaagggcgtcaggtgcagcttttt

SEQ ID NO: 29 - DNA, 62 bp, Artificial
  Synthetic oligonucleotide
gatcaaaaagctgcacctgacgcccttcactgacaggaaggtgaagggcgtcaggtgcagcg

SEQ ID NO: 30 - DNA, 56 bp, Artificial
  Synthetic oligonucleotide
ggaggattgtggccttctttcttcctgtcaaaagaaggccacaatcctcccttttt

SEQ ID NO: 31 - DNA, 62 bp, Artificial
  Synthetic oligonucleotide
gatcaaaaagggaggattgtggccttcttttgacaggaagaaagaaggccacaatcctcccg

SEQ ID NO: 32 - DNA, 56 bp, Artificial
  Synthetic oligonucleotide
gatccaggataacggaggctcttcctgtcaagcctccgttatcctggatccttttt

SEQ ID NO: 33 - DNA, 62 bp, Artificial
  Synthetic oligonucleotide
gatcaaaaaggatccaggataacggaggcttgacaggaagagcctccgttatcctggatccg

SEQ ID NO: 34 - DNA, 62 bp, Artificial
  Synthetic oligonucleotide
ggcaggcgcaatcttcgcatttcttttttccaggaatctagagatatcgagctcaataaaag

SEQ ID NO: 35 - DNA, 69 bp, Artificial
  Synthetic oligonucleotide
aattctttattgagctcgatatctctagattcctggaaaaaagaaatgcgaagattgcgcctgcctgca

SEQ ID NO: 36 - DNA, 33 bp, Artificial
  PCR Primer
gggtctagagccaccatggaagacgccaaaaac

SEQ ID NO: 37 - DNA, 30 bp, Artificial
  PCR Primer
cccgagctccttacacggcgatctttccgc

SEQ ID NO: 38 - DNA, 97 bp, Artificial
  PCR Primer
tgctgttgacagtgagcgcgctcaccatcttcatcaagtatagtgaagccacagatgtatacttgatgaagatggtgagcatgcctactgcctcgga

SEQ ID NO: 39 - DNA, 97 bp, Artificial
  PCR Primer
tgctgttgacagtgagcgcgctgtttacaggcttcatcaatagtgaagccacagatgtattgatgaagcctgtaaacagcttgcctactgcctcgga

SEQ ID NO: 40 - DNA, 97 bp, Artificial
  PCR Primer
tgctgttgacagtgagcgcggagacagtttcagatgattatagtgaagccacagatgtataatcatctgaaactgtctccatgcctactgcctcgga

SEQ ID NO: 41 - DNA, 66 bp, Artificial
  PCR Primer
gctcaccatcttcatcaagtatagtgaagccacagatgtatacttgatgaagatggtgagcttttt

SEQ ID NO: 42 - DNA, 66 bp, Artificial
  PCR Primer
gctgtttacaggcttcatcaatagtgaagccacagatgtattgatgaagcctgtaaacagcttttt

SEQ ID NO: 43 - DNA, 66 bp, Artificial
  PCR Primer
ggagacagtttcagatgattatagtgaagccacagatgtataatcatctgaaactgtctccttttt

SEQ ID NO: 44 - DNA, 55 bp, Artificial
  PCR Primer
gctcaccatcttcatcaagtagaagcttgtacttgatgaagatggtgagcttttt

SEQ ID NO: 45 - DNA, 55 bp, Artificial
  PCR Primer
gctgtttacaggcttcatcaagaagcttgttgatgaagcctgtaaacagcttttt

SEQ ID NO: 46 - DNA, 55 bp, Artificial
  PCR Primer
ggagacagtttcagatgattagaagcttgtaatcatctgaaactgtctccttttt

SEQ ID NO: 47 - DNA, 72 bp, Artificial
  PCR Primer
gatcaaaaagctcaccatcttcatcaagtatacatctgtggcttcactatacttgatgaagatggtgagccg

SEQ ID NO: 48 - DNA, 72 bp, Artificial
  PCR Primer
gatcaaaaagctgtttacaggcttcatcaatacatctgtggcttcactattgatgaagcctgtaaacagccg

SEQ ID NO: 49 - DNA, 72 bp, Artificial
  PCR Primer
gatcaaaaaggagacagtttcagatgattatacatctgtggcttcactataatcatctgaaactgtctcccg

SEQ ID NO: 50 - DNA, 61 bp, Artificial
  PCR Primer
gatcaaaaagctcaccatcttcatcaagtacaagcttctacttgatgaagatggtgagccg

SEQ ID NO: 51 - DNA, 61 bp, Artificial
  PCR Primer
gatcaaaaagctgtttacaggcttcatcaacaagcttcttgatgaagcctgtaaacagccg

SEQ ID NO: 52 - DNA, 61 bp, Artificial
  PCR Primer
gatcaaaaaggagacagtttcagatgattacaagcttctaatcatctgaaactgtctcccg

SEQ ID NO: 53 - DNA, 3074 bp, Artificial
  PCR Primer
gggggtggggagtgttgttaaccggaggggcagccgcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggagactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcctctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcttttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaaaaaaaaaa

SEQ ID NO: 54 - DNA, 3071 bp, Artificial
  PCR Primer
gggggtggggagtgttgttaaccggaggggcagccgcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggagactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcctctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcttttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaaaaaaaaaa

SEQ ID NO: 55 - DNA, 17 bp, Artificial
  PCR Primer
gtcggagtcaacggatt

SEQ ID NO: 56 - DNA, 17 bp, Artificial
  PCR Primer
aagcttcccgttctcag

(19) United States
(12) Patent Application Publication
(10) Pub. No.: US 2011/0206639 A1
(43) Pub. Date: Aug. 25, 2011

(54) REPLICATION COMPETENT VIRUSES CAPABLE OF SILENCING VIRUS INHIBITORY FACTOR EXPRESSION

(76) Inventors: Christie Vermeulen, Amsterdam (NL); Victor Willem van Beusechem, Amstelveen (NL); Jan Eduard Carette, Cambridge, MA (US)

(21) Appl. No.: 12/875,554

(22) Filed: Sep. 3, 2010

Related U.S. Application Data
(63) Continuation-in-part of application No. 12/715,942, filed on Mar. 2, 2010.
Application No. 12/715,942 is a continuation-in-part of application No. 11/545,095, filed on Oct. 9, 2006.
Application No. 11/545,095 is a continuation of application No. PCT/EP05/04152, filed on Apr. 15, 2005.

(30) Foreign Application Priority Data
Apr. 15, 2004 (EP) 04076154.6

(51) Int. Cl. C12N 7/00; C07H 21/02; C12N 5/00; A61K 48/00; A61K 35/00; A61K 35/76; A61P 35/04

(52) U.S. Cl. 424/93.2; 435/235.1; 536/24.5; 435/375; 424/93.6

(57) ABSTRACT

Described is a replication competent virus, being capable to replicate and having lytic capacity in host cells, the virus comprising in the genome thereof, at least one DNA sequence coding for a silencing factor functional in reducing expression of a target gene in the said host cells, operably linked to one or more expression control sequences, functional in the said host cells, and the use thereof in the preparation of a medicament and to the use thereof in a method for lysing host cells expressing a virus inhibitory factor.

### FIELD OF THE INVENTION

The present invention relates to the fields of genetic modification, biotechnology and medicine. In particular, the invention provides recombinant viruses in particular adenoviruses with a potency to lyse cells in which they replicate and to suppress expression of one or more target genes in said cells in which they replicate. The invention furthermore provides recombinant viruses that silence expression of certain genes in certain cells, which causes said viruses to more effectively replicate in and lyse said cells. The invention thus provides more efficient means to eradicate certain populations of cells. The invention furthermore provides methods and means to identify target genes the silencing whereof causes viruses to more effectively replicate in and lyse cells in which they replicate. The invention furthermore provides recombinant viruses that silence expression of said identified target genes in said cells, which causes said viruses to more effectively replicate in and lyse said cells. The invention finds useful applications in the areas of viral vector production, therapeutic target identification, and medical treatments based on inhibition of protein expression and removal of certain cells from a body, such as e.g. cancer cells.

### BACKGROUND OF THE INVENTION

Recombinant viruses are generated from the genome of viruses through genetic engineering. This genetic engineering often involves insertion of heterologous DNA, including but not limited to DNA encoding a therapeutic product, into the adenovirus genome. It is to be understood, however, that the term recombinant virus is also meant to include virus from which parts of the virus genome have been removed without insertion of heterologous DNA. Another example of a recombinant virus is a chimeric virus containing parts of the genomes of different viruses or of different types of the same virus, such as e.g. different serotype viruses or viruses with different host animal species specificities. For the purpose of the invention, two types of recombinant viruses are discriminated, i.e., replication deficient virus and replication competent virus. The present invention relates only to replication competent virus.

Herein, the recombinant virus is exemplified by adenovirus; the term adenovirus can therefore be replaced by any suitable virus known in the art that is capable of infecting and lysing host cells. Non-limiting examples of other suitable viruses are herpes simplex viruses and vaccinia viruses. It is furthermore to be understood that also in other terms defined herein that contain the term adenovirus, such as e.g. “adenovirus inhibitory factor”, the term adenovirus can be replaced by any suitable virus known in the art that is capable of infecting and lysing host cells.

Replication competent adenovirus is defined herein in that the virus comprises, as part of its genome, the function to be replicated in the host cell, wherein replication is solely dependent on the replication functions provided by the virus, in combination with the endogenous cellular machinery of the host cells. The genome of the host cells is therefore free of any exogenous sequences encoding factors that are necessary for viral replication. These factors are provided by the genome of the replication competent virus.

The term “endogenous” means in this respect that the cellular machinery (including the coding sequences therefor), necessary for virus replication, is the naturally present machinery, e.g. not introduced in the cells by manipulation techniques by man. The latter are defined as “exogenous”. The term “function to be replicated” includes the factors such as proteins, encoded by the virus, necessary for replication of the virus in the host cells (herein also referred to as viral replication factors). Said factors may be endogenous for the said virus, but may also be functional analogues, encoded by the viral genome, e.g. in cases wherein the gene encoding the endogenous viral factor is deleted from the viral genome. It is important to note that these factors are encoded by the viral genome and are not to be complemented by exogenous factors encoded in host cells. Thus, viruses, of which the replication is dependent on one or more replication functions, being deleted from the virus, but introduced in the host cell, are defined to be replication deficient, and are therefore not part of the present invention. The invention as claimed relates to replication competent viruses, i.e. wherein the viral genes encoding viral replication factors, essential for regulation of virus replication in the host cells are present on the viral genome.

In a first type of replication competent adenovirus no parts of the adenovirus genome have been removed or said parts of the adenovirus genome that are removed do not include parts that are essential for at least one step of the adenovirus infectious life cycle. This type of recombinant adenovirus is therefore also defined as a genuine replication competent adenovirus that has a capacity to replicate in cells like the parental unmodified adenovirus does. In general, the replication of adenoviruses is restricted to cells of a particular animal species or group of animal species. E.g., recombinant adenoviruses derived from human adenoviruses can only transverse a complete life cycle in human cells, with very inefficient replication occurring at high dose in cells of some other species.

A second type of replication competent adenovirus is the so-called conditionally replicating adenovirus (CRAd). In CRAds one or more parts of the adenovirus genome are removed, including parts that are essential for at least one step of the adenovirus infectious life cycle under certain physiological conditions (herein also “first conditions”) but not under certain other physiological conditions (herein also “second conditions”). Said first and second conditions could, e.g., be dictated by the physiological conditions that exist in a particular type of cells (herein also “first cells”), but not in another type of cells (herein also “second cells”). Such a first type of cell is e.g. a cell derived from a particular type of tissue, where said cell contains a protein that is not or much less present in cells from other tissues (second type of cells) An example of a second type of cell is a cell that has lost proper cell growth control, such as e.g. a cancer cell, where said cell either lacks a protein that is present in cells that have not lost proper cell growth control or where said cell has gained expression (or over-expression) of a protein that is not or much less present in cells that have not lost proper cell growth control. Another example of said second conditions are the conditions that exist in a particular stage of the cell cycle or in a particular developmental stage of the cell, where a certain protein is expressed specifically. Thus, CRAds can be designed such, that replication thereof is enabled in particular cells, such as cancer cells or a particular type of cancer cells, whereas in normal cells, replication of CRAds is not possible. This is known in the art, and reviewed e.g. by Heise and Kirn, J. Clin. Invest. 105 (2000):847-851; Alemany et al., Nat. Biotech. 18 (2000):723-727; Gomez-Navarro and Curiel, Lancet Oncol. 1 (2000):148-158).

In a third type of replication competent adenovirus, parts of the genome are removed that include the function to be replicated in the host cell, but said function is complemented by inserting one or more functional expression cassettes for heterologous proteins that provide said function in the recombinant adenovirus genome. This type of recombinant adenovirus is referred to herein as a heterologously trans-complemented adenovirus, and therefore, is to be regarded as replication competent according to the definition presented herein.

The adenovirus replication process constitutes the following steps: (1) infection of the host cell by binding of the adenovirus particle to the cell surface, internalization and transport towards the cell nucleus, and import of the adenovirus DNA genome into the cell nucleus, (2) expression of adenovirus proteins encoded by the early regions in the adenovirus genome, (3) replication of the adenovirus genome, which marks the transition of the early replication phase to the late replication phase, (4) expression of adenovirus proteins encoded by the late regions in the adenovirus genome, (5) assembly of progeny adenovirus particles and inclusion of progeny adenovirus genomes into these particles, and (6) induction of cell death, leading to release of adenovirus progeny from the cell. During their life cycle, adenoviruses modulate cell death pathways. In different cell lines, p53 dependent as well as p53 independent apoptosis has been documented after adenovirus infection (Teodoro and Branton, J. Virol. 71 (1997):1739-1746; and references therein). During the early replication phase, cell death is suppressed to prevent premature cell death, thereby allowing the adenovirus to complete its life cycle in the cell. In contrast, at late stages of infection cell death and lysis are promoted to release the virus progeny from the cell.

The production of recombinant adenoviruses usually starts with genetic engineering of at least a part of the adenovirus genome by standard molecular biology techniques known in the art. Next, the adenovirus genome, comprised by one or more (in this case overlapping) constructs, is introduced into cells that allow replication of said recombinant adenovirus by DNA transfer methods known in the art. After the recombinant adenovirus has started to replicate in cells into which the recombinant adenovirus genome has been introduced, said recombinant adenovirus can spread to other cells in the culture. The recombinant adenovirus can also be isolated from the culture medium or from lysates of the cells in which said recombinant adenovirus is replicating. The isolated recombinant adenovirus can then be used to re-infect new cells to further propagate and expand said recombinant adenovirus. In addition, said recombinant adenovirus can be administered to an animal or human body to infect cells in vivo. This administration can be done via several routes, including but not limited to direct injection into a tissue, oral administration, injection into the blood circulation, inhalation, injection into a body cavity, and application to the surface of a certain body area. Following infection of said cells in vivo, the recombinant adenovirus can replicate and spread to other cells in vivo, provided that the infected cells support replication of said recombinant adenovirus.

Replication competent adenoviruses will replicate in many different cells in an animal body, provided that they are derived from adenoviruses with the correct species tropism and that said cells express surface receptors for said adenoviruses. Specific cell surface recognition by recombinant adenoviruses including replication competent adenoviruses can be changed by pseudotyping or targeting (Krasnykh et al., Mol. Ther. 1 (2000):391-405; Havenga et al., J. Virol. 76 (2002):4612-4620; Van Beusechem et al., Gene Ther. 10 (2003):1982-1991). CRAds will only replicate in cells in which the particular conditions exist that are required for replication of the CRAd. CRAds are designed to meet the specific requirements for replication in a chosen (first) type of cell and not in other (second) types of cells. This property makes CRAds particularly useful for several embodiments of the present invention where the intend is to treat a disease by specific lytic replication of the recombinant adenovirus according to the invention in diseased cells in an animal or human body resulting in specific removal of said diseased cells from said body.

Replication competent viruses, in particular adenoviruses, are finding increasing utility for the treatment of cancer and other diseases involving inappropriate cell survival. In particular, CRAds have been developed to selectively replicate in and kill cancer cells. Such cancer-specific CRAds represent a novel and very promising class of anticancer agents (reviewed by Heise and Kirn, supra, Alemany et al., supra; Gomez-Navarro and Curiel, supra). The tumor-selective replication of this type of CRAds is achieved through either of two alternative strategies. In the first strategy, the expression of an essential early adenovirus gene is controlled by a tumor-specific promoter (e.g., Rodriguez et al., Cancer Res. 57 (1997):2559-2563; Hallenbeck et al., Hum. Gene Ther. 10 (1999):1721-1733; Tsukuda et al., Cancer Res. 62 (2002):3438-3447; Huang et al., Gene Ther. 10 (2003):1241-1247; Cuevas et al., Cancer Res. 63 (2003):6877-6884). The second strategy involves the introduction of mutations in viral genes to abrogate the interaction of the encoded RNA or protein products with cellular proteins, necessary to complete the viral life cycle in normal cells, but not in tumor cells (e.g., Bischoff et al., Science 274 (1996):373-376; Fueyo et al., Oncogene 19 (2000):2-12; Heise et al., Clin. Cancer Res. 6 (2000):4908-4914; Shen et al., J. Virol. 75(2001:4297-4307; Cascallo et al., Cancer Res. 63 (2003):5544-5550). During their replication in tumor cells CRAds destroy cancer cells by inducing lysis, a process that is further referred to as “oncolysis”. The release of viral progeny from lysed cancer cells offers the potential to amplify CRAds in situ and to achieve lateral spread to neighboring cells in a solid tumor, thus expanding the oncolytic effect. The restriction of CRAd replication to cancer or hyperproliferative cells dictates the safety of the agent, by preventing lysis of normal tissue cells. Currently, CRAd-based cancer treatments are already being evaluated in clinical trials (e.g., Nemunaitis et al., Cancer Res. 60 (2000):6359-6366; Khuri et al., Nature Med. 6 (2000):879-885; Habib et al., Hum. Gene Ther. 12 (2001):219-226).

However, despite very encouraging results from in vitro and animal studies, the anti-cancer efficacy of CRAds as a single agent in humans has been limited (Kirn et al., Nature Med. 4 (1998):1341-1342; Ganly et al., Clin. Cancer Res. 6 (2000):798-806; Nemunaitis et al., Cancer Res. 60 (2000):6359-6366; Mulvihill et al., Gene Therapy 8 (2001):308-315). Thus, there is a clear need in the field of cancer treatment to increase the potency of replication competent adenoviruses as oncolytic agents. This could be achieved by enhancing their replication and lysis capacities.

Several approaches aimed at improving the replication and lysis capacities of replication competent adenoviruses, or at preventing loss of these functions from the wild-type adenovirus have been taken. It has been shown that it is better to retain the adenovirus E3 region in a replication competent adenovirus (Yu et al, Cancer Res. 60 (2000):4200-4203) or, in case most of the E3 region is deleted, to at least retain the gene encoding the E3-11.6 kDa protein (Tollefson et al, J. Virol. 70 (1996):2296-2306; Doronin et al, J. Virol. 74 (2000):6147-6155). In addition, replication and cell lysis of replication competent adenoviruses have been improved by incorporation of cytotoxic genes (Zhang et al, Proc. Natl. Acad. Sci. USA 93 (1996):4513-4518; Freytag et al, Hum. Gene Ther. 9 (1998):1323-1333; Wildner et al, Gene Ther. 6 (1999):57-62). It was also shown that replication competent adenoviruses are more potent in killing cancer cells when they are deleted of the gene encoding the anti-apoptotic E1B-19 kDa protein (Martin Duque et al, Cancer Gene Ther. 6 (1999):554-563; Sauthoff et al, Hum. Gene Ther. 11 (2000):379-388).

Recently, we found that oncolysis and release of adenovirus progeny from infected cancer cells can be accelerated by restoring p53 functions in said cancer cells (van Beusechem et al., Cancer Res. 62 (2002):6165-6171; WO 03/057892, incorporated by reference herein). Said restoring of p53 functions is done by expressing in said cancer cells a restoring factor, i.e. a functional factor of the p53-dependent apoptosis pathway, the function whereof is not or insufficiently expressed in said cancer cells, wherein said restoring factor preferably comprises a protein (WO 03/057892). Hence, said restoring factor is an essential positive component of the p53-dependent apoptosis pathway.

Cancer cells and cell lines are the result of neoplastic transformation. The genetic events underlying neoplastic transformation include activation of proto-oncogenes and inactivation of tumor-suppressor genes. A major player in this respect is the gene encoding the tumor-suppressor protein p53. The p53 protein is the central coordinator of damage-induced cell-cycle checkpoint control. In a perturbed cell, p53 can induce growth arrest and cell death. p53 exerts these effects by functioning as a specific transcription factor that controls the expression of a large panel of genes involved in growth control, DNA repair, cell-cycle arrest, apoptosis promotion, redox regulation, nitric oxide production, and protein degradation (Polyak et al., Nature 389 (1997):237-238; El-Deiry, Sem. Cancer. Biol. 8 (1998):345-357; Yu et al., Proc. Natl. Acad. Sci. USA 96 (1999):14517-14522; Hupp et al., Biochem. J. 352 (2000):1-17; and references therein). The induction of cell death by p53 is mediated at least in part by activation of pro-apoptotic death genes of the bcl-2 family, such as bax, bak, bad, bid, bik, bim, bok, blk, hrk, puma, noxa and bcl-x_{S} (Miyashita and Reed, Cell 80 (1995):293-299; Han et al., Genes Dev. 10 (1996):461-477; Zoernig et al., Biochim. Biophys. Acta 1551 (2001):F1-F37). On the other hand, anti-apoptotic members of the bcl-2 family, such as bcl-2 itself and bcl-x_{L}, bcl-w, bfl-1, brag-1 and mcl-1 inhibit p53-dependent cell death (Zoernig et al., supra). The anti-apoptotic protein Bax Inhibitor-1 (BI-1) suppresses apoptosis through interacting with bcl-2 and bcl-x_{L} (Xu and Reed, Mol. Cell 1 (1998):337-346). The immediate effector proteins of p53 as well as p53 itself target mitochondria, thereby releasing cytochrome c into the cytosol to activate the caspase cascade via the initiator caspase-9/Apaf-1 complex (Juergensmeier et al., Proc. Natl. Acad. Sci. USA 95 (1998):4997-5002; Fearnhead et al., Proc. Natl. Acad. Sci. USA 95 (1998):13664-13669; Soengas et al., Science 284 (1999):156-159; Marchenko et al., J. Biol. Chem. 275 (2000):16202-16212). Negative regulators of the caspase cascade include but are not limited to members of the Inhibitor of Apoptosis Protein (IAP) family of proteins, such as cIAP1, cIAP2, cIAP3, XIAP and survivin (Zoernig et al., supra).

The loss of normal function of p53 is associated with resistance to programmed cell death, cell transformation in vitro and development of neoplasms in vivo. In approximately 50% of human cancers the gene encoding p53 is non-functional through deletion or mutation (Levine et al, Nature 351 (1991):453-456; Hollstein et al, Science 253 (1991):49-53; Chang et al, J. Clin. Oncol. 13 (1995):1009-1022). In many of the other 50% cancer cells that do express wild-type p53 protein, p53 function is still hampered by the action of a “p53 antagonist”. A “p53 antagonist” is defined herein as a molecule capable of inhibiting p53 function. E.g., loss of the tumor-suppressor protein p14ARF or overexpression of MDM2 protein can lead to functional inactivation of p53 by binding to the MDM2 protein and subsequent degradation (Landers et al., Oncogene 9(1994):2745-2750; Florenes et al., J. Natl. Cancer Inst. 86(1994):1297-1302; Blaydes et al., Oncogene 14 (1997):1859-1868; Stott et al, EMBO J. 17 (1998):5001-5014; Schmitt et al, Genes Dev. 19 (1999):2670-2677). Other nonlimiting examples of molecules that promote p53 degradation include Pirh2 (Leng et al, Cell 112 (2003):779-791), COP1 (Dornan et al, Nature 429(2004):86-92) and Bruce (Ren et al, Proc. Natl. Acad. Sci. USA 102 (2005):565-570). Another example is functional inactivation of p53 as a result of the antagonizing binding of human papilloma virus (HPV) E6 protein in cervical carcinomas (Scheffner et al., Cell 63 (1990):1129-1136) or of herpesvirus-8 latency-associated nuclear antigen (LANA) in Kaposi's sarcoma (Friborg et al., Nature 402 (1999):889-894). Yet another example is functional inactivation of p53 as a result of cytoplasmic retention through binding of p53 to Parc (Nikolaev et al., Cell 112 (2003):29-40) or to mot-2/mthsp70/GRP75/mortalin (Wadhwa et al., Exp. Cell Res. 274 (2002):246-253). Furthermore, some molecules can indirectly reduce the amount of functional p53 in a cell and are therefore herein also considered as p53 antagonists, although they are not considered members of the p53 pathway. For example, elevated expression of polo-like kinase-1 (plk-1) decreases p53 stability (Liu and Erikson, Proc. Natl. Acad. Sci. USA 100 (2003):5789-5794). Another example is Wip1/PPM1D, which dephosphorylates MDM2, thereby increasing its stability and affinity for p53, facilitating p53 degradation (Lu et al., Cancer Cell 12 (2007):342-354). The complexity of p53 activity regulation by interaction with other cellular proteins through post-translational modification was reviewed by Lavin and Gueven (Cell Death Diff. 13 (2006):941-950). In addition, even if p53 function itself is intact, p53-dependent cell death can be hampered due to overexpression of anti-apoptotic proteins acting on the p53 pathway down-stream from p53, such as the anti-apoptotic bcl-2 and IAP family members and BI-1. Another example is p73DeltaN, which binds to p53-responsive promoters competing with p53, thereby antagonizing p53-dependent cell death (Kartasheva et al, Oncogene 21 (2002):4715-4727). For the purpose of the invention, said anti-apoptotic proteins acting on the p53 pathway down-stream from p53 are referred to as “p53 pathway inhibitors”. Thus, in many if not all cancers in vivo and cancer-derived or immortalized cell lines in vitro p53-dependent cell death is hampered as a result of one or more lesions in the p53 pathway. These lesions can occur upstream or downstream of p53 and p53 function may be inhibited by a p53 antagonist of p53 pathway inhibitor directly or indirectly.

Loss of p53 function has also been documented in other diseases involving inappropriate cell survival, such as for example rheumatoid arthritis (Firestein et al., J. Clin. Invest. 96 (1995):1631-1638; Firestein et al., Am. J. Pathol. 149 (1996):2143-2151; Firestein et al., Proc. Natl. Acad. Sci. USA 94 (1997):10895-10900) and vascular smooth muscle cell hyperplasia (Speir et al., Science 265(1994):391-394; Kovacs et al., Am J. Pathol. 149 (1996):1531-1539).

Other molecules involved in regulation of programmed cell death include but are not limited to members of the death effector domain protein family (reviewed by Tibbetts et al., Nat. Immunol. 4 (2003):404-409). It is to be understood that many anti-apoptotic proteins known to be important in the regulation of programmed cell death or cancer cell maintenance do not act on the p53 pathway. They are, therefore, not considered members of the p53 pathway. For example, inhibition of cyclin E or of DNA replication initiation proteins or of fatty acid synthase or of PAX2 caused apoptosis in cancer cells (Li et al, Cancer Res. 63 (2003):3593-3597; Feng et al, Cancer Res. 63 (2003):7356-7364; De Schrijver et al, Cancer Res. 63 (2003):3799-3804; Muratovska et al, Oncogene 22 (2003):6045-6053). Therefore, all these targets the inhibition whereof leads to apoptosis and that are not members of the p53 pathway are considered anti-apoptotic proteins for the purpose of the invention. Many genes known to be important in the regulation of programmed cell death or cancer cell maintenance have been shown to be targets for anti-cancer therapy (reviewed by Jansen and Zangemeister-Wittke, Lancet Oncol. 3 (2002):672-683). Several methods have been used successfully to selectively suppress these targets, including expression of dominant-negative proteins, introduction of small inhibitor molecules, RNA antisense expression and RNA interference. The present invention makes use of RNA interference.

RNA interference (RNAi) is a conserved cellular surveillance system that recognizes double-stranded RNA (dsRNA) and activates a sequence-specific degradation of RNA species homologous to the dsRNA (Hannon, Nature 418 (2002):244-251). In addition, RNAi can cause transcriptional gene silencing by RNA-directed promoter DNA methylation and/or histone methylation (Kawasaki and Taira, Nature 431(2004):211-217; Morris et al, Science 305(2004):1289-1292). Furthermore, in some species RNAi has been implicated in programmed DNA elimination and meiotic silencing (reviewed by Matzke and Birchler; Nature Rev. Genet. 6 (2005):24-35). The observation that dsRNA could elicit a potent and specific gene silencing effect was first made in experiments with Caenorhabditis elegans where dsRNA, a byproduct in the generation of antisense RNA, proved to be more effective than antisense RNA itself (Fire et al. Nature 391 (1998):806-811). In retrospect, this observation offered an explanation for the phenomenon of post transcriptional gene silencing frequently encountered in transgenic plants (Baulcombe, Plant Mol. Biol. 32 (1996):79-88). After these initial reports, RNAi-related processes have been described in almost all eukaryotic organisms, including protozoa, flies, nematodes, insects, parasites, and mouse and human cell lines (reviewed in: Zamore, Nat. Struct. Biol. 8 (2001):746-750; Hannon, Nature 418 (2002):244-251; Agrawal et al., Microbiol. Mol. Biol. Rev. 67 (2003):657-685). By now, RNA interference is the most widely used method to specifically downregulate genes for functional studies.

The molecular mechanism of RNAi involves the recognition and cleavage of dsRNA into small interfering RNAs (siRNAs) by the RNase III enzymes Dicer and Drosha (Carmell and Hannon, Nat. Struct. Biol. 11(2004):214-218), the incorporation of the siRNAs in a multiprotein complex called RISC(RNA-induced silencing complex) and the degradation of homologous RNA (Caudy et al., Nature 425 (2003):411-414). The siRNAs perform an essential role in guiding RISC to the target mRNA. siRNAs consist of double-stranded 18-23 nucleotide RNA duplexes carrying 2 nucleotide 3′-OH overhangs that determine in part the efficacy of gene silencing (Elbashir et al., Genes Dev. 15 (2001):188-200). During the incorporation of the siRNA into the RISC complex, the siRNA is unwinded and only one strand is assembled into the active RISC complex. This process is asymmetric in nature and RISC preferentially accepts the strand of the siRNA that presents the less stable 5′ end (Khvorova et al., Cell 115 (2003):209-216; Schwarz et al. Cell 115 (2003):199-208). This has important ramifications in the selection of siRNA sequences because only siRNAs from which the antisense strand (with regard to the targeted mRNA) is assembled into RISC will be effective. Guidelines have been proposed for the selection of highly effective siRNA sequences based on the free energy profile of the siRNA sequences (Khvorova et al., Cell 115 (2003):209-216; Schwarz et al. Cell 115 (2003):199-208; Reynolds et al. Nat. Biotechnol. 22(2004):326-330; Ui-Tei et al., Nucleic Acids Res. 32(2004):936-948).

Application of dsRNA to silence expression of genes in mammalian cells lagged behind due to the occurrence of a general response triggered by dsRNA molecules larger than 30 basepairs. This response is mediated by dsRNA-activated protein kinase (PKR) and 2′,5′ OligoA-synthetase/RNAseL and results in a shut down of translation followed by apoptosis (Kumar and Carmichael, Microbiol. Mol. Biol. Rev. 62 (1998):1415-1434; Gil and Esteban, Apoptosis 5 (2000):107-114). Therefore, initially, application of RNAi by long dsRNA was confined to mammalian cells that lack the PKR response: i.e. embryonic cells (Billy et al., Proc. Natl. Acad. Sci. USA 98 (2001):14428-14433; Svoboda et al., Development 127 (2000):4147-4156). In a breakthrough experiment Elbashir and coworkers showed that chemically synthesized siRNAs, resembling the siRNAs produced by Dicer and Drosha, induced gene specific silencing in cultured mammalian cells without triggering the PKR response (Elbashir et al., Nature 411 (2001):188-200). Synthesized siRNAs are now widely used as tool to study the function of individual genes offering a convenient and rapid method to silence genes (McManus and Sharp, Nat. Rev. Genet. 3 (2002):737-747). However, the transient nature of the silencing effect and the difficulty of delivering synthetic siRNAs in vivo restrict the utility of this approach.

A further means of generating siRNAs is to express small RNA molecules inside the cell either by co-expression of sense and antisense RNAs (Zheng et al., Proc. Natl. Acad. Sci. 101(2004):135-140; Miyagishi et al., Nat. Biotechnol. 20 (2002):497-500; Lee et al., Nat. Biotechnol. 20 (2002):500-505), or as a single transcript that forms a stem-loop structure. The latter RNA molecules are generally referred to as short hairpin RNAs (shRNAs) and typically consist of a 19-29 nucleotide stem containing complementary sense and antisense strands and a loop of varying size. shRNAs generated inside the cell are processed by Dicer to form siRNAs and are capable of inducing RNAi. A variety of promoters have been used to drive the expression of shRNAs. RNA polymerase III promoters are especially suited because they have well-defined initiation sites and a termination site consisting of a stretch of at least 4 consecutive thymidine nucleotides. The RNA polymerase III (polIII) promoters H1, U6, 7SK and tRNA(Val) have been successfully used to express shRNAs (Brummelkamp et al., Science 296 (2002):550-553; Paddison et al., Genes Dev. 16 (2002) 948-958; Kawasaki and Taira, Nucleic Acids Res. 31 (2003):700-707; Koper-Emde et al., Biol. Chem. 385(2004):791-794). In addition, polIII-based drug-inducible shRNA expression cassettes have been developed that permit the conditional suppression of genes in mammalian cells (Wiznerowicz and Trono, J. Virol. 77 (2003):8957-8961; Gupta et al., Proc. Natl. Acad. Sci. USA 101(2004):1927-1932). RNA polymerase II promoters (polII) seemed less suited to express functional shRNAs and initial attempts failed to induce silencing (Paddison et al., Genes Dev. 16 (2002) 948-958). However, successful use of the polII CMV promoter was reported by using a minimal CMV promoter and a modified polyA signal (Xia et al., Nat. Biotechnol. 20 (2002):1006-1010) or by using ribozyme mediated cleavage of the transcript (Kato and Taira, Oligonucleotides 13 (2003):335-343; Shinagawa and Ishii, Genes Dev. 17 (2003):1340-1345). Another possibility is to use the U1 snRNA promoter that has well defined transcription start and termination sites (Denti et al., Mol. Ther. 10(2004):191-199). PolII promoters can also be used if the shRNA is expressed in a precursor format similar to that of microRNAs (miRNAs), the natural cellular inducers of RNAi. miRNA molecules are transcribed by polymerase II as pri-miRNA with a cap and poly-A tail and processed to short, approximately 70-nucleotide stem-loop structures known as pre-miRNA in the cell nucleus. These pre-miRNAs are exported to the cytoplasm and then processed to mature double stranded miRNAs. Successful PolII promoter driven shRNA expression was accomplished in pre-miRNA design as well as pri-miRNA design (e.g., Zeng et al, Methods Enzymol. 392 (2005):9779-9784; Silva et al., Nat. Genet. 37 (2005):1281-1288; Dickins et al., 37 (2005):1289-1295; Stegmeier et al., Proc. Natl. Acad. Sci. USA 102 (2005):13212-13217).

Reports that shRNAs expressed from plasmids could trigger RNAi allowed the use of viral vectors. Retroviral or lentiviral delivery into mammalian cells leads to stable integration of the shRNA-expression cassette in the genome and long-term, sustained gene suppression and is frequently used (e.g., (Brummelkamp et al., Science 296 (2002):550-553; An et al., Hum. Gene Ther. 14 (2003):1207-1212). Adenoviral vectors infect dividing and non-dividing cells but remain episomal. Non-replicating adenoviral vectors expressing shRNAs have been shown to induce silencing of target genes in vitro and in vivo (Xia et al., Nat. Biotechnol. 20 (2002):1006-1010; Arts et al., Genome Res. 13 (2003):2325-2332; Shen et al., FEBS Lett. 539 (2003):111-114; Zhao et al., Gene 316 (2003):137-141; WO 2004/013355). At the time of the priority filing of this application, RNAi with viral vectors had only been done using replication deficient viral vectors. For clarity, it is repeated here that the present invention relates only to replication competent virus. It had not been suggested before to employ RNAi in the context of a replication competent virus because this was not obvious. RNAi has been recognized as a cellular defence mechanism against viral infection and this had led many viruses to evolve molecules that inhibit RNAi (Cullen, Nature Immunol. 3(200):597-599; Roth et al., Virus Res. 102(2004):97-108). Therefore, prior to the present invention there was reason to assume that the process of RNAi would be hampered in cells in which a virus is replicating. The results disclosed herein, which could not be predicted from prior literature, demonstrate that RNAi can be successfully employed in the context of a replication competent virus.

Since then, RNAi has been successfully accomplished in the context of replication competent adenoviruses (e.g., Carette et al., Cancer Res. 64(2004):2663-2667; Zhang et al., Cancer Res. 66 (2006):9736-9743; Yoo et al., Mol. Ther. 15 (2007):295-302; Zheng et al., Cancer Gene Ther. 16 (2009):20-32). Silencing of various different target genes in host cells infected with replication competent adenoviruses expressing shRNA molecules was shown. Thus, it is now established that the RNAi machinery in host cells remains sufficiently intact upon replication competent adenovirus infection to achieve effective target gene silencing.

RNA interference in mammalian cells is now widely used to analyze the function of individual genes. Numerous genes have been successfully silenced in mammalian cells either by transfection of chemically synthesized siRNA or by expression of shRNAs. Classes of genes targeted include genes involved in signal transduction, cell-cycle regulation, development, cell-death, et cetera (Milhavet et al., Pharmacol. Rev. 55 (2003):629-648). Systematic studies for delineating gene function on a genome scale are feasible when large libraries targeting human genes are available. Large libraries of chemically synthesized siRNAs are already commercially available (from Dharmacon and Qiagen), which cause strong, but transient inhibition of gene expression. By contrast, vector-expressed shRNAs can suppress gene expression over prolonged periods. Two research groups (Berns et al., Nature 428(2004):431-437; Paddison et al., Nature 428(2004):427-431) independently constructed and reported on an shRNA-based library covering 7,914 and 9,610 human genes, respectively. Both libraries use polIII promoters and a retroviral vector based on self-inactivating murine-stem-cell-virus. shRNA libraries are now also commercially available in arrayed format (Sigma, Open Biosystems) and in pooled format (Open Biosystems). These siRNA and shRNA libraries are being used to identify gene function in mammalian cells using high-throughput genetic screens (reviewed in e.g. Echeverri and Perrimon, Nature Rev. Genet. 7 (2006):373-384; Iorns et al, Nature Rev. Drug Discov. 6 (2007):556-568). These screens are rapidly advancing knowledge of biological pathways and are yielding new targets for improving therapy of diseases, including cancer. Berns et al (supra) already used their library to identify new components of the p53 pathway by screening for inhibition of cell senescence. Additional members of the p53 pathway were discovered this way by Llanos et al. (Cell Cycle 5 (2006):1880-1885) and Mullenders et al. (PLoS ONE 4 (2009):e4798). It is expected that synthetic lethal high-throughput screenings will be performed to evaluate shRNAs for their ability to kill engineered tumorigenic cells but not their isogenic normal cell counterparts (Brummelkamp and Bernards, Nat. Rev. Cancer 3 (2003):781-789). Thus, identification of selective anti-cancer shRNAs is foreseen.

### BRIEF DESCRIPTION OF THE INVENTION

In many instances it is preferred that a replication competent adenovirus undergoes a rapid life cycle in a host cell. When a replication competent adenovirus is produced or when a replication competent adenovirus is used as a vector to produce a protein in cells, a rapid adenovirus life cycle speeds up the production process. When a replication competent adenovirus is used as a means to kill a population of cells, a rapid life cycle will add to the efficacy of the process. A rapid life cycle is of particular importance for the use of a replication competent adenovirus in vivo. Adenoviruses induce potent immune responses in the body of animals that inactivate said adenoviruses. This limits the duration of in vivo replication of an administered replication competent adenovirus. A faster life cycle will thus allow more cycles of progeny virus production within the time-span between administration and inactivation of the replication competent adenovirus. A situation where a rapid life cycle of a replication competent adenovirus is of particular importance in vivo is in the context of the treatment of a disease involving inappropriate cell survival. A paradigm example of such a disease is cancer. The anticancer potency of a replication competent adenovirus that is administered to a tumor in vivo depends on (1) the efficiency at which the virus disseminates throughout the tumor by producing progeny that can infect neighboring tumor cells, and (2) the efficiency at which the virus kills tumor cells via replication and lysis of the said cells. Lysis of cancer cells is called oncolysis. Thus, a rapid life cycle will result in a faster oncolysis, more cycles of new virus production per time, infection of more tumor cells in time, and, consequently, more effective tumor destruction.

Therefore, a first objective of the present invention is to provide replication competent adenoviruses that have a short replication time in a host cell. Said replication time is understood to mean the time between entry of the replication competent adenovirus into the cell and the release of progeny of said replication competent adenovirus from the cell.

It is a second objective of the present invention to provide replication competent adenoviruses that have a fast lytic capacity. A fast lytic capacity is understood to mean a short time required to lyse a host cell after entry of the replication competent adenovirus into said host cell.

According to the present invention, said short replication time and/or fast lytic capacity of the replication competent adenovirus is brought about by silencing the expression of one or more target genes in the host cell through RNA interference. It is thus to be clearly understood that the present invention utilizes RNA interference to provide said replication competent adenovirus with said short replication time and/or fast lytic capacity.

In preferred embodiments of the invention, said RNA interference is induced by one or more silencing factors that are expressed from the genome of the replication competent adenovirus, where it is further preferred that said silencing factors are shRNA molecules. In a less preferred variation of the invention, said silencing factors consist of double-stranded RNA molecules that are formed in the cell from two RNA molecules expressed from the genome of the replication competent adenovirus.

In further preferred embodiments of the invention, said host cell in which said replication competent adenoviruses have a short replication time and/or a fast lytic capacity is a cell expressing one or more adenovirus inhibitory factors. In one preferred embodiment of the invention, said host cell is a cell with a defect in a cell death pathway. In another preferred embodiment of the invention, said host cell is hampered in the p53-dependent cell death pathway. Said cell is preferably a human cell. Non-limiting examples of host cells according to the invention are cancer or tumor cells, arthritic cells and hyperproliferative vascular smooth muscle cells.

It is to be understood that a “defect in a cell death pathway” includes inability of a cell to respond to a loss of cell-cycle checkpoint control, to DNA damage and/or to oncogene expression by executing programmed cell death. Said defect in a cell death pathway causes “inappropriate cell survival”.

In one variation of the invention, said host cell is a cell that is being cultured in vitro. In another variation of the invention, said host cell is a cell in an animal body, where it is preferred that said animal body is a human body.

In a preferred embodiment of the invention, said fast lytic capacity is the result of restoration of said defect in a cell death pathway. In a further preferred embodiment of the invention, said restoration is in the p53-dependent cell death pathway.

In a variation of the invention, the replication competent adenovirus expressing one or more silencing factors from its genome according to the invention furthermore expresses a functional factor of the p53-dependent apoptosis pathway according to the invention disclosed in WO 03/057892, included by reference herein. This variation of the invention thus provides the combined effect of restoring the p53-dependent apoptosis pathway by expressing said functional factor and of silencing one or more adenovirus inhibitory factors by said one or more silencing factors.

Thus, the replication competent adenoviruses according to the invention are capable of replicating in a host cell and expressing one or more silencing factors. Said silencing factors silence the expression of a host cell factor capable of inhibiting said short replication time and/or fast lytic capacity of replication competent adenoviruses in said host cell. Said host cell factor that inhibits said short replication time and/or fast lytic capacity of the replication competent adenovirus is further referred to as an “adenovirus inhibitory factor”. The character of said adenovirus inhibitory factor is not dictated in any way other than it being expressed in the host cell and being capable of inhibiting adenovirus replication or adenovirus-induced host cell lysis. Non-limiting examples of said adenovirus inhibitory factor are p53 antagonists and p53 pathway inhibitors. Further non-limiting examples of said adenovirus inhibitory factor are anti-apoptotic proteins that are not members of the p53 pathway. Other non-limiting examples of said adenovirus inhibitory factor are molecules that are identified by using a method to identify an adenovirus inhibitory factor according to the invention (infra). The amount of silencing brought about by said one or more silencing factors should be sufficient to decrease the amount of adenovirus inhibitory factor to a level that allows said short replication time and/or fast lytic capacity of the replication competent adenovirus of the invention in said cell.

In a variation of the invention, the replication competent adenovirus expressing one or more silencing factors from its genome according to the invention furthermore comprises one or more modifications in its genome known in the art that change its tropism, i.e. host cell recognition specificity.

In another variation of the invention, the replication competent adenovirus expressing one or more silencing factors from its genome according to the invention furthermore comprises one or more modifications in its genome known in the art that reduce its immunogenicity.

In yet another variation of the invention, the replication competent adenovirus expressing one or more silencing factors from its genome according to the invention furthermore comprises one or more modifications in its genome known in the art that restrict its replication in a first type of cells but not in a second type of cells.

In yet another variation of the invention, the replication competent adenovirus expressing one or more silencing factors from its genome according to the invention furthermore comprises one or more modifications in its genome known in the art that augment its replication and/or lytic capacity.

As outlined above, it is to be clearly understood that the terms “replication competent” and being “capable of replicating in a host cell” mean that said recombinant adenoviruses alone are capable of completing their infectious life cycle in said host cell with the aid of the endogenous machinery of the said host cell, without a need to provide any functions encoded by any removed parts of the genome of said recombinant adenoviruses by other means, such as the provision thereof in the genome of the host cell. The said recombinant adenovirus is a replication competent adenovirus, preferably a conditionally replicating adenovirus or a heterologously trans-complemented adenovirus. Said recombinant adenovirus is not a replication deficient adenovirus.

The term “silencing factor” means an RNA molecule capable of decreasing expression of a target gene through the process of RNA interference. The said RNA molecule preferably comprises a double stranded portion, preferably having a length of at least 19 nucleotides (per strand). The double stranded portion preferably has a length of less than 30 nucleotides, and the said RNA molecule preferably comprises a short hairpin RNA as outlined above (shRNA), or comprises a double-stranded structure consisting of two different RNA molecules having complementary regions of preferably the above length. In the latter case, the said RNA molecules can be transcribed from different promoters. The said RNA molecules are preferably susceptible to the action (i.e. the recognition and cleavage of dsRNA) of the above-mentioned RNAse III enzymes, resulting in the above-discussed siRNAs. The term “target gene” is understood to indicate that the process of decreasing expression of said gene occurs with specificity.

The invention also provides formulations comprising the replication competent adenoviruses according to the invention that can be used to preserve said replication competent adenoviruses and to administer said replication competent adenoviruses to cells. In one variation, the formulations are used to administer said replication competent adenoviruses to cells in vitro, in another variation the formulations are used to administer said replication competent adenoviruses to cells in vivo.

The invention furthermore provides methods to administer the formulations according to the invention to cells, leading to infection of said cells with the replication competent adenoviruses of the invention. In one variation, the methods are used to administer said formulations to cells in vitro, in another variation the methods are used to administer said formulations to cells in vivo.

The invention also provides compositions of the replication competent adenoviruses according to the invention and cells in which the replication competent adenoviruses according to the invention induce accelerated cell lysis and/or a faster release of virus progeny, compared to replication competent adenoviruses that are not expressing one or more silencing factors according to the invention. In a preferred variation of the invention, said cells are cancer cells and said cell lysis is oncolysis. In a further preferred variation of the invention, said cells are human cells.

In another embodiment, the invention provides compositions of the replication competent adenoviruses according to the invention and tumors in which the replication competent adenoviruses according to the invention induce accelerated cell lysis and/or a faster release of virus progeny, compared to replication competent adenoviruses that are not expressing one or more silencing factors according to the invention. In this aspect of the invention, it is preferred that said accelerated cell lysis and/or a faster release of virus progeny results in an accelerated lateral spread by said replication competent adenoviruses from infected cells to neighboring cells in said tumors, compared to replication competent adenoviruses that are not expressing one or more silencing factors according to the invention, where it is furthermore preferred that said accelerated cell lysis, faster release of virus progeny and/or accelerated lateral spread lead to a more effective destruction or growth inhibition of said tumors. In a preferred variation of the invention, said tumors are growing in an animal body. In a further variation, said animal body is a human body.

The invention furthermore provides methods to construct the replication competent adenoviruses according to the invention and to produce the formulations and compositions according to the invention.

The invention furthermore contemplates the use of the replication competent adenoviruses, methods and formulations according to the invention for the treatment of a disease which involves inappropriate cell survival, where it is preferred that said disease is a disease in a human being. In a particular embodiment of the invention said disease is cancer.

The invention furthermore provides methods to identify adenovirus inhibitory factors that are expressed in cells. In preferred embodiments, said cells are cells with a defect in a cell death pathway and said cells are human cells.

The invention furthermore provides methods to identify and to select silencing factors that are useful for being expressed from the genome of the replication competent adenoviruses according to the invention.

Hereinafter, in several embodiments of the invention a number of ways to provide said replication competent adenoviruses, silencing factors, formulations, methods, compositions, and uses are given. It is to be clearly understood that the description is not meant to in any way limit the scope of the invention as was explained in general terms above. Skilled artisans will be able to transfer the teachings of the present invention to other replication competent adenoviruses, silencing factors, formulations, methods, compositions, and uses that are not mentioned specifically herein without departing from the present invention.

It is also to be understood that the invention includes all combined uses of the replication competent adenoviruses, silencing factors, formulations, methods and compositions of the invention together with other methods and means to kill a population of cells, including but not limited to irradiation, introduction of genes encoding pro-apoptotic proteins, toxic proteins, such as for example toxins or prodrug converting enzymes, and administration of chemical compounds, antibodies, receptor antagonists, and the like.

### DESCRIPTION OF THE FIGURES

FIG. 1. (A) Conditionally replicating adenoviruses encoding shRNAs silence the expression of a target gene. CRAd-shRNA induced silencing of firefly luciferase in four human cell lines. Cells were infected with the indicated CRAds immediately followed by a transfection with the reporter plasmid phAR-FF-RL and silencing was analyzed 30 hours after infection. Ratios of firefly luciferase to Renilla luciferase activity were normalized to the ratio obtained after infection with the Ad5-Δ24E3 control virus. Data shown are the mean results+SD of a representative experiment performed in triplicate. (B) Time course of CRAd-shRNA-induced silencing of firefly luciferase in A549 cells. Cells were either infected with Ad5-Δ24E3 or with Ad5-Δ24E3.Ff1-R and analyzed at various time points after infection. At each time point, the ratio of firefly luciferase activity to Renilla luciferase activity obtained with Ad5-Δ24E3.Ff1-R was normalized to that of Ad5-Δ24E3 virus. Data shown are the mean results±SD of a representative experiment performed in triplicate. (C) CRAd-shRNA induced silencing of firefly luciferase is sequence specific. A549 cells were infected with the indicated CRAds immediately followed by a transfection with the reporter plasmid phAR-FF-RL or the mutated plasmid phAR-FF*-RL and analyzed as in (A). Data shown are the mean results±SD of a representative experiment performed in triplicate.

FIG. 2. Influence of cell-cycle inhibitor apigenin on luciferase expression from recombinant adenoviruses. A549 cells were infected with Ad5Δ24-SA-luc, Ad5Δ24-CMV-Luc or Ad5ΔE1-CMV-Luc at MOI 20 PFU/cell and cultured in the presence of increasing amounts of apigenin. Thirty-two hours post infection cells were lysed and the luciferase activities were determined. Data shown are the mean results±SD of a representative experiment performed in triplicate.

FIG. 3. Restoration of p53 function in cells expressing the p53 antagonist HPV18E6 by a replication competent adenovirus expressing p53 and an shRNA directed against HPV18E6. HeLa HPV18-positive cervical cancer cells were transfected with p53 reporter construct PG13-Luc and 24 hours later infected with AdΔ24, AdΔ24-p53, or AdΔ24.p53(L).H1_{—}18E6. Seventy-two hours post infection cells were lysed and the luciferase activities were determined. Values were normalized on the basis of mock-infected controls. Data shown are the mean results of a representative experiment performed in triplicate.

FIG. 4. Specific augmentation of adenovirus lytic replication in cells expressing the p53 antagonist HPV18E6 by a replication competent adenovirus expressing p53 and an shRNA directed against HPV18E6. MDA-MB-231 HPV-negative cells (A), SiHa HPV16-positive cells (B) and HeLa HPV18-positive cells (C) were infected with AdΔ24, AdΔ24-p53, or AdΔ24.p53(L).H1_{—}18E6 at a range of virus doses as indicated. After 20 days culture, remaining cells that survived adenovirus lytic replication were stained. MOI, multiplicity of infection, i.e., number of infectious viruses per cell in the inoculum.

FIG. 5. SYVN1 silencing induces p53 activity and increases oncolysis in A549 cells. P53 activation upon silencing of indicated genes was determined in (A) a PG13LUC derivative of U2OS and (B) two independent A549-PG13LUC derivative cell lines (cell clone 1, black bars; cell clone 2, white bars). Cancer cells stably expressing PG13-Luc were transfected with siRNAs against 17 putative p53 inhibitors. Seventy-two hours later, luciferase expression was measured. To determine p53-dependent transcriptional activation, the fold induction in luciferase activity was calculated over negative control siRNA (NT#1). Results are the mean of 3 independent experiments of triplicate wells; error bars are SD. Asterisk in B marks sample with significant difference in relative luciferase activity compared to siCONTROL-1 negative control siRNA (NT#1) (p≦0.005 Student's t test). C) Effect of AdWT on cell viability upon p53 inhibitor silencing. A549 cells were transfected with siRNAs against 17 putative p53 inhibitors and infected with AdWT. Cell viability was determined by measuring CellTiter-Blue conversion, 3 (black bars) and 5 (white bars) days after infection. Mean of 3 independent experiments in triplicate; error bars are SDs.

FIG. 6. Restoring p53 function by SYVN1 silencing increases oncolysis. A549 cells were transfected with SYVN1 or control siRNAs or left untransfected; and infected with AdWT or not. Cell viability was determined by measuring CellTiter-Blue conversion on days 2 to 8 after infection. Three independent experiments in triplicate are shown in panels A, B and C; error bars are SDs.

FIG. 7. SYVN1 silencing accelerates AdWT-induced death of A549 cells. A549 cells were transfected with SYVN1 or control siRNAs or left untransfected; and infected with AdWT or not. Viable cells were counted on days 1 to 5 after infection. Data shown is the mean of 2 independent experiments each done in triplicate; error bars are SDs.

FIG. 8. Effect of SYVN1 silencing on AdWT propagation. A549 cells were transfected with SYVN1 or control siRNAs or not transfected (mock); and subsequently infected with AdWT. Cells and medium were harvested at days 3, 4 and 5 after infection and infectious virus titers were determined. Data are the mean of 3 independent experiments in triplicate; error bars are SDs. Asterisk marks significant difference in virus titer compared to mock and siRNA control (p≦0.05 Student's t test).

FIG. 9. Silencing of SYVN1 using shRNA enhances AdWT oncolysis. A549 cells were stable transfected with three different GIPZ SYVN1 shRNAmir lentiviral vectors or with a GIPZ lentiviral empty vector control; and infected with AdWT at MOI 100 or left uninfected. Cell viability was determined by BCA protein assay on days 2 to 5 after infection. Data are the mean of 3 to 4 independent experiments in triplicate; error bars are SDs. Significant difference in protein concentrations after AdWT infection were observed between A549 cells stably expressing SYVN1 shRNA with SEQ ID NO 38 or SEQ ID NO 39 and A549 cells expressing the empty vector control, 3, 4 and 5 days after infection; and between A549 cells stably expressing SYVN1 shRNA with SEQ ID NO 40 and A549 cells expressing the empty vector control, 4 and 5 days after infection (p≦0.05 Student's t test).

FIG. 10. List of sequences.

FIG. 11. Enhanced adenovirus induced cell kill by incorporation of expression cassettes for shRNAs against SYVN1 in conditionally replication competent adenovirus. Oncolysis by AdΔ24E3-shSYVN1-1 and AdΔ24E3-shSYVN1-4 was enhanced compared to AdΔ24E3. A549 cells were infected with AdΔ24E3, AdΔ24E3-shSYVN1-1 or AdΔ24E3-shSYVN1-4 at MOI 10 (A) and 100 (B) or left uninfected. Cell viability was determined by BCA protein assay on day 3 after infection. Data are the mean of triplicate wells of one experiment. Protein concentrations were substantially lower after infection with AdΔ24E3-shSYVN1-1 and Ad24E3-shSYVN1-4 than after infection with Ad24E3.

### DETAILED DESCRIPTION OF THE INVENTION

The invention and preferred embodiments appear from the appended claims.

In one embodiment, the present invention provides a replication competent adenovirus capable of replicating in a host cell, characterized in that the said virus comprises at least one DNA sequence coding for a silencing factor, in particular a short hairpin RNA, capable of reducing expression of a target gene in the said host cell, said DNA sequence being functionally linked to regulatory DNA sequences in such a manner that said silencing factor is expressed in said host cell into which said replication competent adenovirus is introduced.

In a second embodiment, the present invention provides a replication competent adenovirus capable of replicating in a host cell, characterized in that the said virus comprises at least one DNA sequence coding for a silencing factor, in particular a short hairpin RNA, capable of reducing expression of an adenovirus inhibitory factor in the said host cell, said DNA sequence being functionally linked to regulatory DNA sequences in such a manner that said silencing factor is expressed in said host cell into which said replication competent adenovirus is introduced.

In a variation of the present invention, a replication competent adenovirus is provided that comprises more than one DNA sequence coding for a silencing factor capable of reducing expression of a target gene in the said host cell functionally linked to regulatory DNA sequences in such a manner that said silencing factor is expressed in a cell into which said replication competent adenovirus is introduced. In one variation, at least two DNA sequences of said more than one DNA sequence each encode for a different silencing factor capable of reducing expression of a different target gene.

This variation of the invention is used to silence expression of more than one target gene in the said host cell. In another variation, at least two DNA sequences of said more than one DNA sequence each encode for a different silencing factor capable of reducing expression of the same target gene. This variation of the invention is used to more effectively silence expression of said target gene in said host cell.

In another embodiment of the present invention, a replication competent adenovirus is provided that comprises at least one DNA sequence coding for a silencing factor capable of reducing expression of a target gene in the said host cell, said DNA sequence being functionally linked to regulatory DNA sequences in such a manner that said silencing factor is expressed in said host cell into which said replication competent adenovirus is introduced, and furthermore comprises at least one open reading frame for a restoring factor capable of restoring the p53-dependent apoptosis pathway in the said host cell according to WO 03/057892, said open reading frame being functionally linked to regulatory DNA sequences in such a manner that said restoring factor is expressed in said host cell into which said recombinant adenovirus is introduced.

Useful silencing factors that are to be expressed from the genome of the replication competent adenoviruses of the invention in host cells in which said replication competent adenoviruses are replicating form double-stranded RNA molecules that are processed by Dicer in said host cells to form siRNAs that are capable of inducing RNAi in said host cells.

The virus can be replicated in the host cell in several ways as discussed above; the skilled person will be aware of suitable replication strategies useful for the practice of the invention.

The recombinant adenovirus according to the invention is a replication competent adenovirus, such as (1) a genuine replication competent adenovirus (2) a conditionally replicating adenovirus, or (3) a heterologously trans-complemented adenovirus, or (4) a two-component replication competent, heterologously trans-complemented, or conditionally replicating adenovirus consisting of as a first component a recombinant adenovirus that comprises at least one DNA sequence coding for a silencing factor capable of reducing expression of a target gene in the said host cell, said DNA sequence being functionally linked to regulatory DNA sequences in such a manner that said silencing factor is expressed in said host cell into which said recombinant adenovirus is introduced and as a second component a replication competent, heterologously trans-complemented, or conditionally replicating adenovirus, where a single-component replication competent adenovirus according to possibilities 1, 2 or 3 is preferred.

Non-limiting examples of conditionally replicating adenoviruses according to the invention are derived from adenoviruses with controlled expression of at least one essential early adenovirus gene by a tumor-specific promoter, including but not limited to those described by Rodriguez et al. (Cancer Res. 57 (1997):2559-2563), Hallenbeck et al. (Hum. Gene Ther. 10 (1999):1721-1733), Tsukuda et al. (Cancer Res. 62 (2002):3438-3447), Huang et al. (Gene Ther. 10 (2003):1241-1247) and Cuevas et al. (Cancer Res. 63 (2003):6877-6884), or adenoviruses with mutations in viral genes to abrogate the interaction of the encoded proteins with cellular proteins, necessary to complete the viral life cycle in normal cells, but not in tumor cells, including but not limited to those described by Heise et al. (Nature Med. 6 (2000):1134-1139), Balague et al. (J. Virol. 75 (2001):7602-7611), Howe et al. (Mol. Ther. 2 (2000):485-494), Fueyo et al. (Oncogene 19 (2000):2-12), Shen et al. (J. Virol. 75 (2001):4297-4307) and Cascallo et al. (Cancer Res. 63 (2003):5544-5550), or adenoviruses comprising both types of modifications. A non-limiting example of a heterologously trans-complemented adenovirus according to the invention is derived from a recombinant adenovirus with a functionally deleted E1 region that expresses the HPV E6 and E7 proteins (Steinwaerder et al., Mol. Ther. 4 (2001):211-216).

In one embodiment, the replication competent adenovirus according to the invention is characterized in that said silencing factor is capable of reducing expression of a p53 antagonist, including but not limited to MDM2, Pirh2, COP1, Bruce, HPV-E6, Parc, mortalin and plk-1.

In another embodiment, the replication competent adenovirus according to the invention is characterized in that said silencing factor is capable of reducing expression of a p53 pathway inhibitor, including but not limited to BI-1, p73DeltaN and the anti-apoptotic bcl-2 and IAP family members (supra).

In another embodiment, the replication competent adenovirus according to the invention is characterized in that said silencing factor is capable of reducing expression of an adenovirus inhibitory factor, wherein said adenovirus inhibitory factor is characterized in that it delays replication of a replication competent adenovirus lacking said silencing factor in a host cell or that it delays lysis of a host cell in which a replication competent adenovirus lacking said silencing factor is replicating. It is to be clearly understood that in this embodiment of the invention the character of said adenovirus inhibitory factor is not dictated in any way other than it being expressed in said host cell and being capable of inhibiting adenovirus replication or adenovirus-induced host cell lysis. Said ‘being capable of inhibiting adenovirus replication or adenovirus-induced host cell lysis’ means that in the presence of a sufficient amount of said adenovirus inhibitory factor in a host cell 10% completion of the process of adenovirus replication or 50% completion of the process of adenovirus-induced host cell lysis is delayed in time by at least 10% compared to the otherwise identical situation in the absence of said adenovirus inhibitory factor, wherein it is preferred that said process is delayed by at least 20% and wherein it is more preferred that said process is delayed by at least 50%. In a variation of the invention, said adenovirus inhibitory factor is identified by a method according to the invention (infra).

Control sequences operably linked to the sequences encoding the silencing factor include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers. The promoter is typically selected from promoters that are functional in mammalian cells, although promoters functional in other eukaryotic cells may be used. The type of promoter is chosen to accomplish a useful expression profile for said silencing factor in the context of said replication competent adenovirus. As described supra, RNA polymerase III promoters, including but not limited to the U6, 7SK, H1 and tRNA(Val) promoters are especially suitable for the invention, but other promoters are not excluded.

In one variation of the invention, said DNA sequence coding for a silencing factor is functionally linked to one or more control sequences, i.e. regulatory DNA sequences, in such a manner that said silencing factor is expressed ubiquitously in a cell into which said replication competent adenovirus is introduced. In another variation of the invention, said DNA sequence coding for a silencing factor is functionally linked to one or more control sequences, i.e. regulatory DNA sequences, in such a manner that said silencing factor is only expressed or is expressed at a higher level in a cell into which said replication competent adenovirus is introduced under certain conditions that can be modulated by an external signal, where the term “external” means having its origin outside of the DNA fragment encompassing said DNA sequence coding for a silencing factor and said regulatory DNA sequences. In this aspect of the invention, the expression of said silencing factor is driven by a so-called regulatable or inducible promoter. In yet another variation of the invention, said DNA sequence coding for said silencing factor is functionally linked to regulatory DNA sequences in such a manner that said silencing factor is only expressed during the late phase of adenovirus replication in a cell into which said replication competent adenovirus is introduced. In this aspect of the invention, it is preferred that expression of said silencing factor is driven by the adenovirus major late promoter (MLP), preferably the endogenous MLP.

The invention does not dictate the site of insertion of said DNA sequence coding for a silencing factor functionally linked to regulatory DNA sequences in the genome of said replication competent adenovirus; said insertion may be at any location in said genome that does not inhibit replication of said replication competent adenovirus in said cell into which said replication competent adenovirus is introduced and where endogenous expression cassettes in said genome do not interfere with proper expression of said silencing factor. In non-limiting examples of the invention, said insertion is a replacement of the adenovirus E3-region or insertion between the E4 promoter and the right-hand ITR. DNA constructs to generate recombinant adenoviruses with insertions in the E3-region are known in the art, including but not limited to pBHG10 and pBHG11 (Bett et al., Proc. Natl. Acad. Sci. USA 91(1994):8802-8806), a DNA construct to generate recombinant adenoviruses with insertions between the E4 promoter and the right-hand ITR is described in Example 1 and insertions at other sites within the adenovirus genome can be made using standard molecular biology methods known in the art. In specific situations, it is preferred for proper expression of said open reading frame to shield said DNA sequence coding for a silencing factor functionally linked to regulatory DNA sequences from other regulatory DNA sequences present in said adenovirus genome by flanking said DNA sequence coding for a silencing factor functionally linked to regulatory DNA sequences by so-called insulator elements (Steinwaerder and Lieber, Gene Therapy 7 (2000):556-567). In another variation of the invention, said DNA sequence coding for a silencing factor is inserted in place of an adenovirus gene, where it is preferred that said adenovirus gene is expressed during the late phase of adenovirus replication, and where it is further preferred that said adenovirus gene is functionally linked to the endogenous MLP.

It is to be understood that the invention also includes replication competent adenoviruses according to the invention that additionally comprise one or more further modifications to change their characteristics, including but not limited to a change of tropism through pseudotyping or targeting, such as e.g. described by Krasnykh et al. (Mol. Ther. 1 (2000):391-405), Suzuki et al. (Clin. Cancer Res. 7(20012):120-126), Van Beusechem et al. (Gene Ther. 10 (2003):1982-1991) and Havenga et al. (J. Virol. 76 (2002):4612-4620); expression of one or more transgenes, such as e.g. a gene encoding a cytokine, a pro-apoptotic protein, an anti-angiogenic protein, a membrane fusogenic protein or a prodrug converting enzyme; or mutations that increase their replication potential, such as e.g. retention of the E3 region (Suzuki et al., Clin. Cancer Res. 8 (2002):3348-3359) or deletion of the E1B-19K gene (Sauthoff et al. Hum. Gene Ther. 11 (2000):379-388), or their replication selectivity for a certain type of cells, including but not limited to the modifications to make CRAds (supra), or that reduce their immunogenicity (i.e., their potency to induce an immune response when introduced into an animal body), such as e.g. retention of the E3B region (Wang et al., Nature Biotechnol. 21 (2003):1328-1335).

The replication competent adenoviruses of the invention are produced using molecular biology, virology and cell biology methods known in the art. A way to produce the replication competent adenoviruses according to the invention is described in detail in the examples section. It is to be understood, however, that this description is not meant in any way to limit the scope of the invention. Those skilled in the art will be able to derive the replication competent adenoviruses of the invention using other methods or by using variations of the methods described herein.

In another embodiment, the invention provides methods to identify an adenovirus inhibitory factor that is a useful silencing target for the replication competent adenoviruses of the invention, by comparing the replication time or the lytic capacity of a replication competent adenovirus in a first type of host cells, in which said adenovirus inhibitory factor is silenced by a silencing factor, to the replication time or the lytic capacity of said replication competent adenovirus in a second type of host cells, in which said adenovirus inhibitory factor is not silenced by a silencing factor, but that are otherwise identical to said first type of host cells, and by observing a shorter replication time or faster lytic capacity in said first type of host cells than in said second type of host cells. Differences in replication time of replication competent adenoviruses in two types of host cells can be measured for example by using a replication competent adenovirus that expresses a marker gene, such as e.g. the firefly luciferase gene, under regulation of the MLP. In this case, luciferase expression in said host cells depends on replication of said replication competent adenovirus. Typically, luciferase expression by such a virus that is replicating in host cells will increase by more than 100-fold or even more than 1000-fold until replication is completed. A convenient measure for adenovirus replication rate in this case is the time required to reach 10% of the maximum value of luciferase activity. Luciferase activity is monitored in time by a method known in the art, including but not limited to the Luciferase Chemiluminescent Assay System (Promega) or the ReportaLight Plus Kit (Cambrex), and a first type of host cells is identified in which the luciferase activity increases faster than in said second type of host cells. Faster in this case means in at least 10% less time, preferably at least 20% less time and more preferably at least 50% less time. The factor that is being silenced in said first type of host cells but not in said second type of host cells is then identified as being an adenovirus inhibitory factor. Differences in lytic capacity can be measured for example by monitoring lysis of said host cells infected with a replication competent adenovirus in time, using one of many methods known in the art, including but not limited to the ToxiLight BioAssay (Cambrex), and identifying a first type of host cells that lyses faster than said second type of host cells. A convenient measure for adenovirus lytic capacity in this case is the time required to lyse 50% of the infected cells, i.e., the time required to reach 50% of the maximum value of the marker compound measured in the cytotoxicity assay used. Also in this case, faster means in at least 10% less time, preferably at least 20% less time and more preferably at least 50% less time. The factor that is being silenced in said first type of host cells but not in said second type of host cells is then identified as being an adenovirus inhibitory factor. In a variation of this embodiment, said first type of host cells contain more than one silencing factor, wherein said silencing factors each have a different target sequence on the same target gene.

In a variation of the invention, the RNA interference by the silencing factor in said methods to identify an adenovirus inhibitory factor is done by transfecting siRNA into said first type of host cells. In another variation, the RNA interference by the silencing factor in said method to identify an adenovirus inhibitory factor is done by transfecting said first type of host cells with a plasmid vector expressing shRNA. In yet another variation, the RNA interference by the silencing factor in said method to identify an adenovirus inhibitory factor is done by transducing said first type of host cells with a viral vector expressing shRNA. Non-limiting examples of viral vectors useful in this variation of the invention include vectors derived from retrovirus, lentivirus, adenovirus, herpes simplex virus, simian virus 40, Eppstein Barr virus, vaccinia virus and adeno associated virus.

In a variation of the invention, said methods to identify an adenovirus inhibitory factor are done in a functional genomics high-throughput format wherein said silencing factor is part of a library of silencing factors comprising an array of silencing factors with different target specificities. In a preferred variation, each component of said array comprises more than one silencing factor, wherein each silencing factor in one component of said array has a different target sequence on the same target gene.

In another embodiment, the invention provides methods to select a silencing factor useful for incorporation in replication competent adenoviruses according to the invention, by executing a method to identify an adenovirus inhibitory factor according to the invention and selecting a silencing factor that after being introduced in a host cell causes a shorter replication time or faster lytic capacity of a replication competent adenovirus.

In a variation of the invention, said methods to select a silencing factor useful for incorporation in replication competent adenoviruses according to the invention are done in a functional genomics high-throughput format wherein said silencing factor is part of a library of silencing factors comprising an array of silencing factors with different target specificities. In a preferred variation, each component of said array comprises more than one silencing factor, wherein each silencing factor in one component of said array has a different target sequence on the same target gene. In this variation, said methods comprise a first step in which a component of the array is selected that comprises one or more silencing factors useful for the invention and a second step in which a silencing factor useful for the invention is selected from said more than one silencing factors present in said selected component of the array.

It is to be understood that the methods to identify an adenovirus inhibitory factor or to select a silencing factor useful for incorporation in replication competent adenoviruses according to the invention do not necessarily require a priori knowledge of the nature of said adenovirus inhibitory factor or silencing factor, nor of the biological process by which said adenovirus inhibitory factor inhibits adenovirus replication or adenovirus-induced host cell lysis. The simple fact that said adenovirus inhibitory factor delays adenovirus replication or adenovirus-induced host cell lysis or that said silencing factor accelerates adenovirus replication or adenovirus-induced host cell lysis make them a useful adenovirus inhibitory factor or a useful silencing factor for incorporation in replication competent adenoviruses according to the invention, respectively.

The invention also provides formulations comprising the replication competent adenoviruses according to the invention that can be used to preserve said replication competent adenoviruses and to administer said replication competent adenoviruses to cells. Said formulations preferably consist of said replication competent adenovirus and a diluent. Said diluent allows storage of said replication competent adenovirus for extended time and/or administration of said replication competent adenovirus to cells in culture and/or cells in an animal body, where it is preferred that said animal body is a human body. It is preferred that said diluent allows storage under lyophilized conditions. It is also preferred that said diluent allows both storage and administration of said replication competent adenovirus to cells in culture and/or cells in an animal body. It is to be understood that “to allow storage” means that during storage of said formulation the capability of said replication competent adenovirus to infect a cell is retained with a half-life higher than one week, where it is preferred that said half-life is more than one month, and where it is most preferred that said half-life is more than 6 months. Said storage may be at any temperature below 40° C., but it is preferred that said temperature is between 1° C. and 10° C., or that said temperature is below minus 60° C. It is to be understood that said administration to cells in culture and/or cells in an animal body means that said formulation and said cells are brought together resulting in introduction of said replication competent adenovirus into said cells. It is preferred that said diluent is not toxic to said cells and to said animal body. The invention does not dictate the exact composition of said diluent, but several useful diluents for the purpose of the invention are known in the art. Non-limiting examples of diluents useful in the invention are disclosed in WO 03/057892, incorporated by reference herein. Optionally, said diluent may be further supplemented with additional constituents to increase physical stability of said replication competent adenovirus during storage, to increase said introduction into said cells, or to improve the administration of said recombinant adenoviruses to said cells in an animal body. Said constituents may be different for each specific use of said formulation. Non-limiting examples of said additional constituents are disclosed in WO 03/057892, incorporated by reference herein. Those skilled in the art will be able to define by proper investigation useful diluents and supplements to prepare a formulation according to the invention that results in the introduction of said replication competent adenovirus into cells for each particular use of the invention and each particular method of administration according to the invention.

The invention furthermore provides methods to administer the formulations according to the invention to cells, leading to introduction of the replication competent adenoviruses of the invention into said cells. In one variation, the methods are used to administer said formulations to cells in vitro, in another variation the methods are used to administer said formulations to cells in vivo. The methods according to the invention do not differ in any way from those known in the art to administer other recombinant adenoviruses to cells. In general, the replication competent adenoviruses of the invention are diluted to reach a useful concentration in a diluent according to the invention. In general, said diluent is isotonic to the conditions in an animal body, but in some cases it may be desired to use a diluent at a non-isotonic concentration. Said useful concentration of said replication competent adenovirus will be different for each different use of the invention. Skilled artisans will be able to determine said useful concentration by experimentation. Said formulation is brought into contact with said cells under either static conditions, such as in the case of administration to cells in culture or in the case of injection into an animal tissue, or under dynamic conditions, such as in the case of injection into the blood circulation of an animal body. Said formulation and said cells are brought into contact at a temperature between 0° C. and 40° C., where it is preferred that said temperature is between 30° C. and 40° C. In case said formulation is administered into an animal body, it is preferred that said formulation and said cells are brought into contact at the existing temperature in said animal body. In one variation of the invention, said administration is done at an ambient atmospheric pressure. In another variation of the invention, said administration is done at a pressure above atmospheric pressure. In another variation of the invention, said administration is done by very slow infusion, also known as Convection-Enhanced Delivery (Voges et al., Ann. Neurol. 54 (2003):479-487; Bankiewicz et al., Exp. Neurol. 164 (2000):2-14). Said contact is maintained for a time period sufficiently long to allow introduction of said replication competent adenoviruses into said cells.

The invention also provides compositions of a replication competent adenovirus that comprises at least one open reading frame for a silencing factor according to the invention and cells in which said replication competent adenovirus replicates. Said replication competent adenoviruses have a host range that allows replication in said cells. In a preferred variation of the invention, said cells are cells that have lost capacity to respond to a loss of cell-cycle checkpoint control by undergoing programmed cell death. In particular examples of this variation of the invention, said cells are rheumatoid arthritis cells or cancer cells. For the purpose of the invention, the terms “cancer cells” and “tumor cells” are defined as cells, having lost proper cell growth control, leading to uncontrolled growth and/or replication of the said cells in e.g. a mammalian body, or to accelerated growth/replication or immortality in vitro. Thus, the term includes malignant, premalignant and benign cancer cells. In a not mutually excluding preferred variation of the invention, said cells are human cells. In another not mutually excluding variation of the invention, said cells are cells in an animal body, where it is preferred that said animal body is a human body. The compositions of the invention are obtained by administering a formulation containing a replication competent adenovirus according to the invention to said cells by means of a method according to the invention.

In one embodiment of the invention, said cells that are part of a composition according to the invention, are cells in a solid tumor. In one variation of this embodiment, said tumor is maintained in culture in vitro. In this variation of the invention, said tumor may be artificially derived from cancer cells, such as for example a cell line-derived spheroid, or said tumor may be derived from an explant of a tumor in an animal body. In another variation of this embodiment, said tumor is present in an animal body. In this variation of the invention, said tumor may be surgically implanted into said animal body or said tumor may have arisen from said animal body. In the latter case, it is preferred that said animal body is a human body. In this embodiment of the invention, it is preferred that said short replication time and/or fast lytic capacity result in an accelerated lateral spread by said replication competent adenoviruses from infected cells to neighboring cells in said tumor, compared to replication competent adenoviruses lacking the silencing factor according to the invention. In this aspect of the invention, it is furthermore preferred that said short replication time, fast lytic capacity and/or accelerated lateral spread lead to a more effective destruction or growth inhibition of said tumor.

In another embodiment of the invention, said cells that are part of a composition according to the invention, are rheumatoid synovium cells. In one variation of this embodiment, said rheumatoid synovium cells are maintained in culture in vitro. In another variation of this embodiment, said rheumatoid synovium cells are present in an animal body, where it is preferred that said rheumatoid synovium cells are present in a chronically inflamed joint and where it is furthermore preferred that said animal body is a human body. In this embodiment of the invention, it is preferred that said short replication time and/or fast lytic capacity result in an accelerated lateral spread by said replication competent adenoviruses from infected cells to neighboring cells in said inflamed joint, compared to replication competent adenoviruses lacking the silencing factor according to the invention. In this aspect of the invention, it is furthermore preferred that said short replication time, fast lytic capacity and/or accelerated lateral spread lead to a more effective destruction or growth inhibition of said rheumatoid synovium cells.

In yet another embodiment of the invention, said cells that are part of a composition according to the invention, are vascular smooth muscle cells. In one variation of this embodiment, said vascular smooth muscle cells are maintained in culture in vitro. In another variation of this embodiment, said vascular smooth muscle cells are present in an animal body, where it is preferred that said vascular smooth muscle cells are present in an area of intimal hyperplasia, such as e.g. in atherosclerosis, restenosis or vascular graft occlusion, and where it is furthermore preferred that said animal body is a human body. In this embodiment of the invention, it is preferred that said short replication time and/or fast lytic capacity result in an accelerated lateral spread by said replication competent adenoviruses from infected cells to neighboring cells in said area of intimal hyperplasia, compared to replication competent adenoviruses lacking the silencing factor according to the invention. In this aspect of the invention, it is furthermore preferred that said short replication time, fast lytic capacity and/or accelerated lateral spread lead to a more effective destruction or growth inhibition of said vascular smooth muscle cells.

The invention furthermore contemplates the use of the replication competent adenoviruses, methods and formulations according to the invention for the treatment of a disease which involves inappropriate cell survival, where it is preferred that said disease is a disease in a human being. A treatment according to the invention will comprise administration of a replication competent adenovirus according to the invention, in a formulation according to the invention, to diseased cells in an animal body using a method according to the invention. In a particular embodiment of the invention said disease is cancer and said diseased cells are cancer cells, where it is preferred that said cancer cells are part of a solid tumor or a tumor metastasis. Depending on the type of disease and the nature of the diseased cells, a useful replication competent adenovirus, a useful formulation and a useful route of administration will be chosen. With respect to said replication competent adenovirus, a useful silencing factor may be chosen on the basis of prior investigation, but preferably also on the basis of knowledge of the genetic background of said disease in general, or more preferably of the genetic background of said diseased cells in particular. A useful formulation and route of administration will be chosen on the basis of knowledge on the localization of said diseased cells in said animal body, the characteristics of said diseased cells and the characteristics of other cells present in the part of said animal body to which said formulation is administered. Non-limiting examples of said route of administration include direct injection into a tissue containing diseased cells, for example by convection-enhanced delivery, oral administration, injection into the blood circulation, inhalation, injection into a body cavity, such as the pleural or peritoneal cavity, a joint, or a brain ventricle, injection into the lumen of a part of the gastro-intestinal or urogenital tract, and application to the surface of a certain body area, such as the skin or the otolaryngeal mucosa, for example by means of a mouth wash. If said route of administration is via injection into the blood circulation, it is preferred that said injection is done into an artery that leads to a part of said animal body that contains said diseased cells.

The invention furthermore contemplates that a treatment of a disease according to the invention is combined with other methods and means to kill a population of diseased cells known in the art, including but not limited to irradiation, introduction of genes encoding toxic proteins, such as for example diphteria or pseudomonas toxin, or pro-apoptotic proteins such as Apoptin or TNF-Related Apoptosis-Inducing Ligand, or prodrug converting enzymes like thymidine kinase, cytosine deaminase or carboxylesterase, or cytokines like interleukin-2, interleukin-12 or GM-CSF, or anti-angiogenic products like endostatin or angiostatin, or fusogenic membrane proteins such as e.g. those derived from Human Immunodeficiency Virus, coronavirus or Gibbon Ape Leukemia Virus and administration of chemical compounds, antibodies, receptor antagonists, signaling inhibitors, molecules inhibiting protein-protein interactions, such as e.g. interaction between p53 and a p53 antagonist or between a component of the p53 pathway and a p53 pathway inhibitor, and the like. It is anticipated that such a combined treatment may result in a more effective killing of said population of diseased cells than either treatment alone. In addition, one treatment may potentiate the effect of the other treatment. For example, irradiation and certain chemical compounds are known to induce programmed cell death. Thus, such treatments may potentiate the efficient cell lysis and virus progeny release of a replication competent adenovirus that restores programmed cell death by silencing an inhibitor of programmed cell death according to the invention.

The invention thus relates to a replication competent adenovirus, being capable to replicate and having lytic capacity in host cells, the virus comprising in the genome thereof, at least one DNA sequence coding for a silencing factor functional in reducing expression of a target gene in the said host cells, operably linked to one or more expression control sequences, functional in the said host cells.

In a preferred embodiment the silencing factor comprises double stranded RNA. The length of the double strand preferably comprises per strand at least 19 nucleotides and less than 30 nucleotides. A preferred RNA is an RNA molecule, wherein the said RNA molecule comprises a hairpin RNA. In one embodiment the invention provides a replication competent adenovirus, being capable to replicate and having lytic capacity in host cells, the virus comprising in the genome thereof, at least one DNA sequence coding for a protein expression repression factor functional in reducing expression of a target gene in the said host cells, operably linked to one or more expression control sequences, functional in the said host cells. The protein expression repression factor decreases the expression of a target protein in a cell. The protein expression repression factor is preferably an RNAi, an siRNA and/or an shRNA. In another preferred embodiment said protein expression repression factor is a protein capable of selectively decreasing expression of a target protein in a host cell. Selectively here has the meaning that expression of said target protein is decreased more than that expression of the majority of the other proteins in the host cells is decreased. In this embodiment the invention provides a replication competent virus, being capable to replicate and having lytic capacity in host cells, the virus comprising in the genome thereof, at least one DNA sequence coding for a protein expression repression factor capable of selectively decreasing expression of a target protein in a host cell, operably linked to one or more expression control sequences, functional in the said host cells. A preferred example of such a factor comprises an intracellular antibody that is specific for said target protein. In other preferred embodiments said factor comprises a dominant-negative protein, a small inhibitor molecule, an RNA antisense nucleic acid, an RNA ribozyme, a recombinant zinc finger protein and/or an RNA interference molecule. The present invention preferably makes use of RNA interference.

Said antisense nucleic acid preferably comprises a stretch of more than 50 nucleic acid molecules that are antisense to and can base pair with an RNA transcript from the target gene. Expression of antisense RNA often leads to the formation of double stranded RNA molecules, comprising the antisense RNA and the endogenous sense mRNA. This double stranded RNA molecule prevents the mRNA from being translated into protein. Said RNA ribozyme is preferably a full length hammerhead ribozyme. Said recombinant zinc finger protein is capable of binding specifically to a promoter region or enhancer region of the target gene, thereby inhibiting or preventing transcriptional activation of said target gene, for example by competing with a positively acting transcription factor. Said zinc finger protein comprises a sequence-specific zinc finger DNA binding domain and preferably a negative acting transcription domain (transcriptional repressor domain) which acts in a dominant way to inhibit or prevent transcription of said target gene such as, for example, a Krüppel-associated box domain. In case of a protein expression repression factor, the target protein is preferably encoded by SYNV1, MDC1 and/or PPM1D. Preferably said target protein is encoded by SYNV1. A preferred example of a protein expression repression factor is a silencing factor.

Preferably said replication competent virus is a human adenovirus, preferably of serotype 5. Further preferred is a replication competent virus, wherein the virus is a conditionally replicating adenovirus. A virus according to the invention preferably carries a mutation in the E1A region encompassing at least a part of the CR2 domain of E1A, preferably a deletion encompassing amino acids 122 to 129 (LTCHEAGF) of E1A.

The target gene preferably encodes an adenovirus inhibitory factor. Said adenovirus inhibitory factor is preferably an anti-apoptotic protein. Preferably said adenovirus inhibitory factor is a p53 antagonist or p53 pathway inhibitor. Said virus inhibitory factor is preferably chosen from the group consisting of MDM2, Pirh2, COP1, Bruce, HPV-E6, herpesvirus-8 LANA, Parc, Mortalin, Plk-1, BI-1, p73DeltaN, bcl-2, bcl-xL, bcl-w, bfl-1, brag-1, mcl-1, cIAP1, cIAP2, cIAP3, XIAP, MDM4, cullin 4a (CUL4A), cullin 7 (CUL7), HECT, UBA and WWE domain containing 1 (Huwe1; also termed Mule or ARF-BP1), synoviolin, SET and MYND domain containing 2 (Smyd2; also termed KMT3C), Proteasome activator complex subunit 3 (PMSE3), nuclear receptor subfamily 4, group A (NR4A1), protein phosphatase 1, magnesium-dependent, delta isoform (PM1D), topoisomerase I-binding protein (TOPORS), sirtuin (silent mating type information regulation 2 homolog) 2 (SIRT2), mediator of DNA-damage checkpoint 1 (MDC1) and survivin.

In a preferred embodiment the replication competent adenovirus further comprises the coding sequence of at least one restoring factor functional in restoring the p53 dependent apoptosis pathway in the said host cells, operably linked to one or more expression control sequences, functional in the said host cells.

Said host cells preferably are human cells, preferably chosen from the group, consisting of cancer cells, arthritic cells or vascular smooth muscle cells.

The invention further relates to the use of a replication competent virus according to the invention in a medicament. Said use preferably is for the manufacture of a medicament for suppressing uncontrolled cell growth, in particular malignant cell growth.

The invention also relates to a method for lysing host cells expressing an adenovirus inhibitory factor, comprising the steps of infecting the said host cells with an adenovirus having lytic capacity in the said host cells, replicating the said adenovirus within the said host cells, further comprising the step of providing, in the virus genome at least one DNA sequence coding for a silencing factor, functional in reducing expression of the said adenovirus inhibitory factor in the said host cells, the said at least one DNA sequence capable to be expressed in the host cells upon infection thereof by the said virus. The host cells in a preferred method are infected with a replication competent virus according to the invention.

A preferred method according to the invention comprises the step of subjecting said host cells to irradiation, a toxic chemical compound, a molecule inhibiting protein-protein interaction or inhibiting a signaling pathway, a protein, including but not limited to an antibody, a pro-apoptotic protein, an anti-angiogenic protein and a fusogenic membrane protein, or of introducing a gene encoding said protein, operably linked to one or more expression control sequences, functional in the said host cells. Said host cells are preferably present in an animal body, preferably a human body.

The invention also relates to a method for treatment of a subject body suffering from a condition involving body cells with inappropriate cell survival, comprising the step of administering to the said subject body an effective amount of a replication competent (adeno)virus according to the invention, where it is preferred that said subject body is a human body.

A preferred condition that is treated with a virus of the invention is chosen from the group consisting of cancer, arthritis, in particular rheumatoid arthritis, or vascular smooth muscle cell hyperplasia.

The invention also relates to a method for identifying a silencing factor functional in reducing expression of adenovirus inhibitory factor being expressed in host cells, comprising the steps of creating two types of the said host cells, wherein the first type expresses a silencing factor and the second type does not express said silencing factor, infecting said two types of host cells with an adenovirus, having lytic capacity in the said host cells, replicating the said adenovirus in the said two types of host cells, comparing the rate of replication of the said adenovirus in the said two types of host cells or comparing the rate of lysis of the said two types of host cells, wherein a faster replication of the said virus in the first type of host cells than in the second type of host cells or a faster lysis of the first type of host cells than of the second type of host cells identifies said silencing factor functional in reducing expression of said adenovirus inhibitory factor in said host cells.

A preferred method for identifying a adenovirus inhibitory factor being expressed in host cells comprises the steps of creating two types of the said host cells, wherein the first type expresses a silencing factor and the second type does not express said silencing factor, infecting said two types of host cells with a adenovirus, having lytic capacity in the said host cells, replicating the said adenovirus in the said two types of host cells, comparing the rate of replication of the said adenovirus in the said two types of host cells or comparing the rate of lysis of the said two types of host cells, wherein a faster replication of the said adenovirus in the first type of host cells than in the second type of host cells or a faster lysis of the first type of host cells than of the second type of host cells identifies said silencing factor functional in reducing expression of said adenovirus inhibitory factor in said host cells, and using the identified silencing factor to characterize said adenovirus inhibitory factor.

Said first type of host cells in a preferred method of the invention is a component of an array of first types of host cells, further characterized in that each component of said array expresses a different silencing factor. Said host cells in a preferred method of the invention are human cells, preferably chosen from the group, consisting of cancer cells, arthritic cells or vascular smooth muscle cells.

In a further embodiment, the invention relates to a replication competent adenovirus, being capable of replicating and having lytic capacity in host cells, the virus comprising in the genome thereof, at least one DNA sequence coding for a silencing factor functional in reducing expression of a target gene in the said host cells, said virus further provided with one or more expression control sequences that mediate expression of the DNA sequence in the host cell.

The term “silencing factor” means an RNA molecule capable of selectively decreasing expression of a target protein in a host cell, where selectively means that expression of said target protein is decreased more than expression of the majority of other proteins is decreased in said host cell. The term “protein” means to include complete proteins, as well as peptides or functional fragments of proteins or peptides.

A silencing factor is preferably an RNA molecule capable of decreasing expression of a target gene, encoding a virus inhibitory factor, preferably an adenovirus inhibitory factor. Said virus inhibitory factor is preferably an anti-apoptotic protein such as, preferably, a p53 antagonist or p53 pathway inhibitor. Thusfar, none of the reported screens for regulators of p53 (supra) have identified SYVN1. SYVN1 (NCBI gene ID 84447), also called HRD1, encodes synovial apoptosis inhibitor 1 or synoviolin, an endoplasmic reticulum (ER)-resident E3 ubiquitin ligase. Synoviolin was found highly expressed in synoviocytes of patients with rheumatoid arthritis and the SYVN1 gene was cloned from rheumatoid synovial cells (Amano et al., Genes Dev. 17 (2003):2436-2449). Overexpression of synoviolin in transgenic mice led to advanced arthropathy caused by reduced apoptosis of synoviocytes (Amano et al., Genes Dev. 17 (2003):2436-2449). Yamasaki et al. (EMBO J. 26 (2007):113-122) demonstrated that synoviolin sequestrates p53 in the cytoplasm and targets p53 for ubiquitination, reducing its cellular level and biological functions. Among the ubiquitin ligases currently known to inhibit p53, synoviolin is unique because of its ER localization and its independency from other ligases and transcriptional regulation by p53. Normal expression levels of synoviolin as analyzed by immunohistochemistry are high in parts of the brain, in glandular cells of parts of the gastrointestinal tract and in the tonsil (www.proteinatlas.org). Elevated expression of synoviolin has been reported in the hippocampus in Alzheimer's disease (Hou et al., J. Neurosci. Res. 84 (2006):1862-1870) and in substantia nigra dopaminergic neurons in Parkinson's disease (Omura et al., J. Neurochem. 99 (2006):1456-1469); and synoviolin was found to protect cells against death induced by Huntington protein (Yang et al., Exp. Cell Res. 313 (2007):538-550). Synoviolin expression was, as expression of many other genes, also analysed in cancer tissues, there synoviolin expression is variable, with high expression found in breast cancer, head and neck cancer, liver cancer, melanoma and thyroid cancer; and moderate to high in colorectal and urothelial cancers (www.proteinatlas.org). A role for synoviolin in the etiology or pathophysiology of cancer has until the present invention not been established.

It was surprisingly established by the present inventors that synoviolin functions as a virus inhibitory factor in cancer cells. Therefore, a further preferred silencing factor according to the invention is functional in reducing expression of synoviolin in a host cell. In another preferred embodiment said replication competent adenovirus comprises a DNA sequence coding for a factor functional in reducing expression of synoviolin in a host cell, operably linked to one or more expression control sequences, functional in the said host cell. Said factor can be a protein such as an intracellular antibody specific for synoviolin. A preferred example of such a factor comprises an intracellular antibody that is specific for said target protein. In other preferred embodiments said factor comprises a dominant-negative protein, a small inhibitor molecule, an RNA antisense nucleic acid, an RNA ribozyme, a recombinant zinc finger protein and/or an RNA interference molecule. In a preferred embodiment said factor is a silencing factor for synoviolin.

A silencing factor according to the invention preferably mediates siRNA-mediated silencing. This can be achieved in a host cell by providing the host cell with sense and antisense RNAs that are complementary and are able to form a double stranded molecule by base pairing. Alternatively, a host cell can be provided with a transcript that is able to form a stem-loop structure, also referred to as short hairpin RNA (shRNA). In a preferred embodiment, the at least one DNA sequence in an adenovirus according to the invention codes for a silencing factor comprising a double stranded RNA molecule. Said silencing factor preferably comprises a short hairpin RNA (shRNA). It is further preferred that the length of the double stranded region of the shRNA molecule is between 19 nucleotides and 30 nucleotides per strand. The double stranded portion of the shRNA may be derived from any part of the synoviolin mRNA such as, for example, the human synoviolin mRNA of SEQ ID NO 53 and/or SEQ ID NO 54. A preferred shRNA that mediates silencing of synoviolin comprises a sequence selected from SEQ ID NO 38-46. A preferred replication competent virus may comprise in the genome thereof, more than one DNA sequences coding for different silencing factors that are functional in reducing expression of synoviolin in the host cells, operably linked to one or more expression control sequences, functional in the said host cells. The at least one DNA sequence coding for a silencing factor functional in reducing expression of synoviolin thus may comprise any part of SEQ ID NO 53 and/or SEQ ID NO 54, preferably one or more of SEQ ID NO 38-46. In this variation, each DNA sequence encodes for a different silencing factor capable of reducing expression of synoviolin. This variation of the invention is preferred to more effectively silence expression of synoviolin in said host cell.

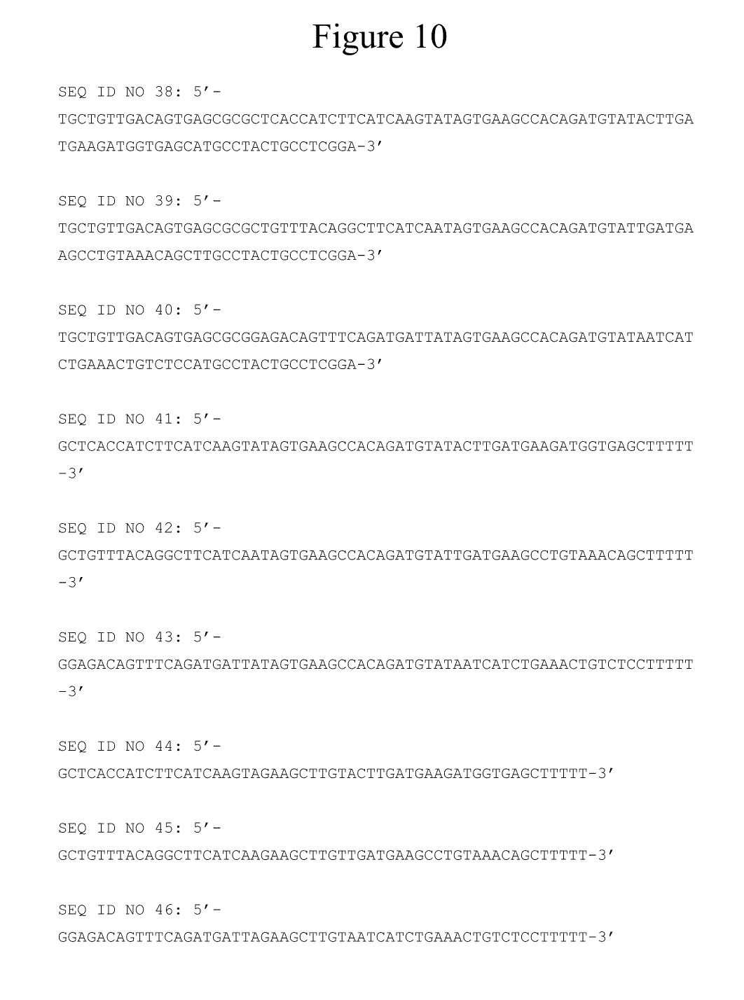

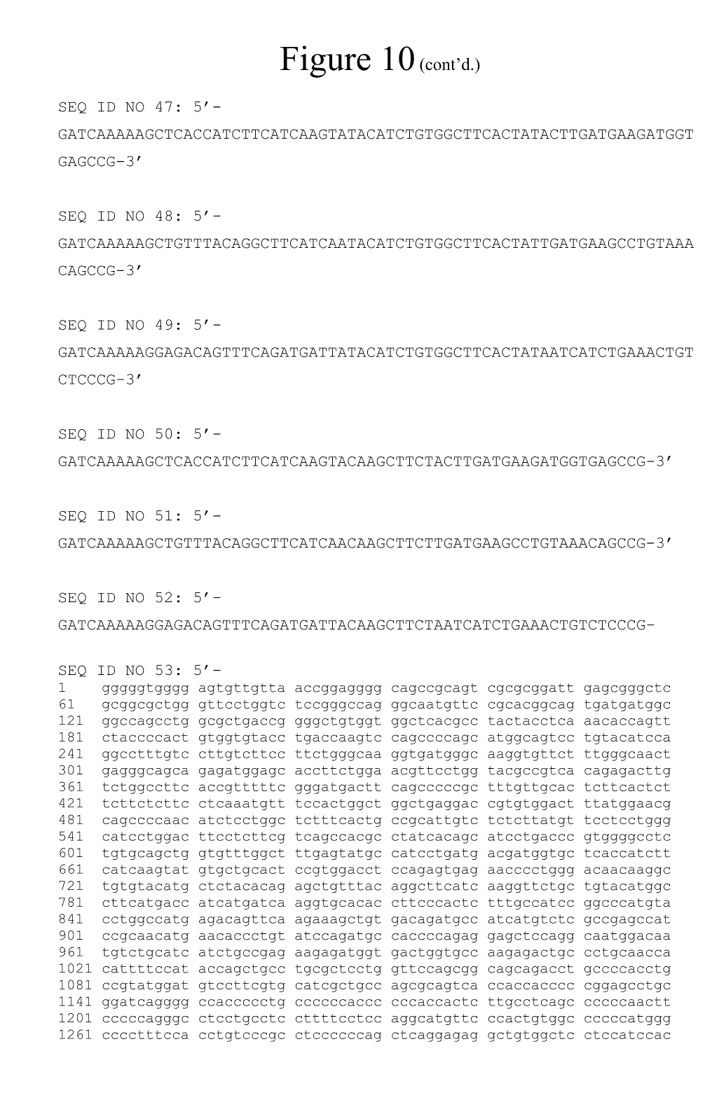

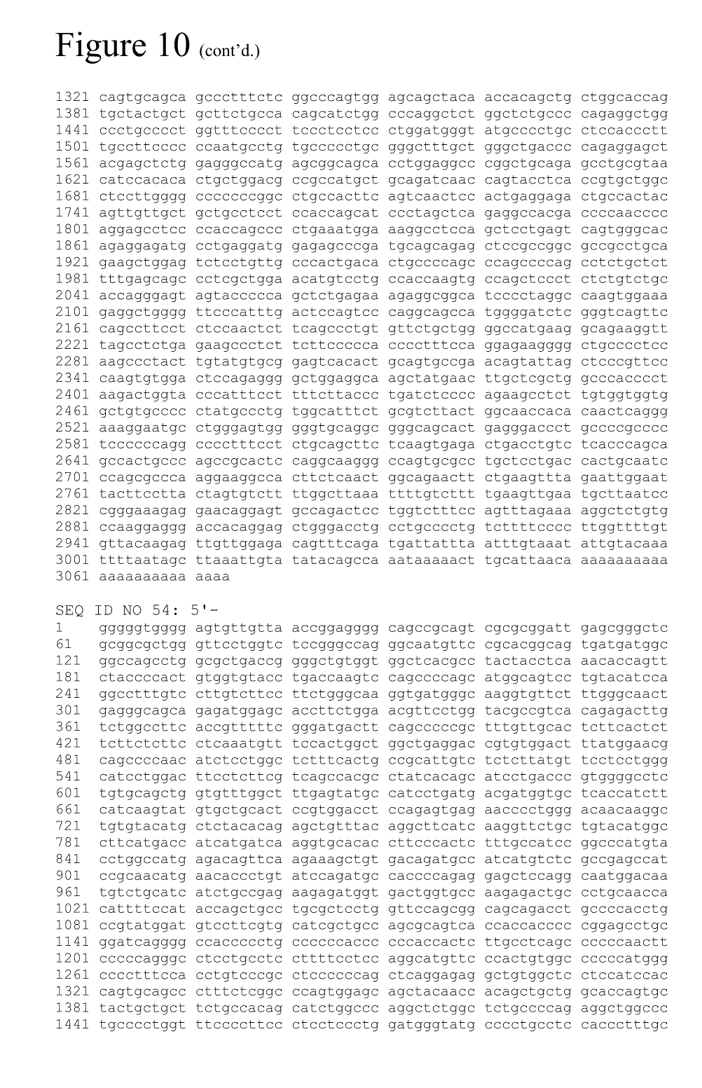

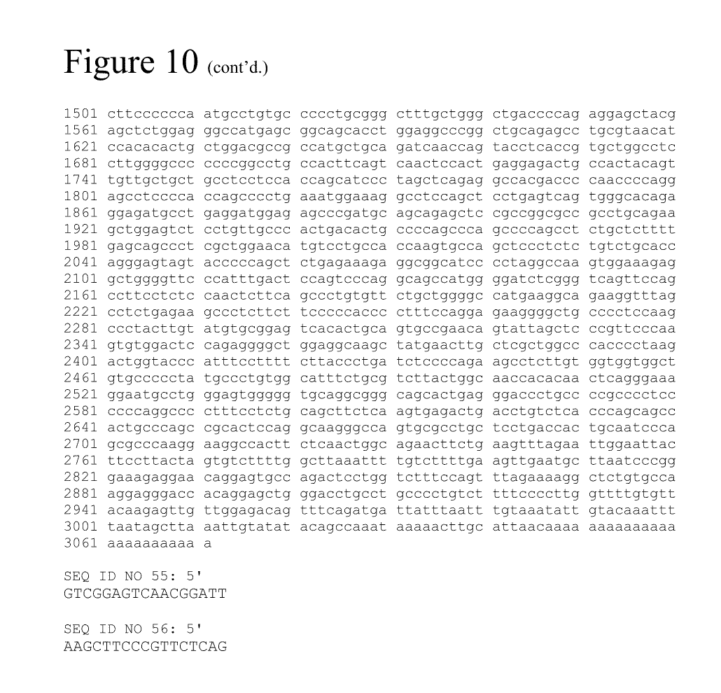

Variations and modifications of the shRNAs can be introduced into them. The shRNAs may have a somewhat shorter and/or somewhat larger region of overlap with the target synoviolin mRNA. Thus also provided the present invention is an shRNA as depicted in FIG. 10 as SEQ ID NO: 38-46, wherein the region of overlap with the target synoviolin mRNA is reduced and/or increased by 1, 2 or 3 nucleotides. Further provided is an adenovirus of the invention comprising an expression cassette encoding an shRNA of FIG. 10 depicted as SEQ ID NO: 38-46, wherein the region of overlap with the target synoviolin mRNA is reduced and/or increased by 1, 2 or 3 nucleotides. Further provided is a shRNA of FIG. 10 depicted as SEQ ID NO: 38-46 comprising 1 or 2 nucleotide alterations with respect tot the target sequence. Said alterations preferably are introduced into both strands of the target recognition sequence in the stem of the shRNA to allow base pairing of introduced nucleotides. Allowing base pairing thus leads to respectively 2 or 4 alterations in the shRNA (Amarzguioui et al. 2003. Nucleic Acids Research 31: 589). For example, the shRNAs comprising a sequence selected from SEQ ID NO 38-46 may comprise one or two nucleotide alterations, preferably G/C transversions, in the target recognition region which is derived from the synoviolin mRNA. In addition, the skilled person will understand that alterations in the loop sequences of the shRNAs, which connect the 5′ target recognition region and the complementary 3′ target recognition region, are well tolerated. It is preferred that alterations in the loop sequences do not result in additional base pairing and do not affect base pairing of stem sequence. Also the exact sequence of a leader, when present, is not that critical and allows sequence variation. Also for leader sequence variation it is preferred that alterations do not result in additional base pairing and do not affect base pairing of stem sequence.

The skilled person will understand that a replication competent virus of the invention may comprise more than one DNA sequences coding for different silencing factors that are functional in reducing expression of synoviolin and of one or more other genes selected from MDM2, Pirh2, COP1, Bruce, HPV-E6, herpesvirus-8 LANA, Parc, Mortalin, Plk-1, BI-1, p73DeltaN, bcl-2, bcl-xL, bcl-w, bfl-1, brag-1, mcl-1, cIAP1, cIAP2, cIAP3, XIAP, MDM4, cullin 4a (CUL4A), cullin 7 (CUL7), HECT, UBA and WWE domain containing 1 (Huwe1; also termed Mule or ARF-BP1), synoviolin, SET and MYND domain containing 2 (Smyd2; also termed KMT3C), Proteasome activator complex subunit 3 (PMSE3), nuclear receptor subfamily 4, group A (NR4A1), protein phosphatase 1, magnesium-dependent, delta isoform (PM1D), topoisomerase I-binding protein (TOPORS), sirtuin (silent mating type information regulation 2 homolog) 2 (SIRT2), mediator of DNA-damage checkpoint 1 (MDC1) and survivin in the host cells, operably linked to one or more expression control sequences, functional in the said host cells. In addition, a replication competent virus of the invention may comprise two or more DNA sequences coding for different silencing factors that are functional in reducing expression of two or more genes selected from MDM2, Pirh2, COP1, Bruce, HPV-E6, herpesvirus-8 LANA, Parc, Mortalin, Plk-1, BI-1, p73DeltaN, bcl-2, bcl-xL, bcl-w, bfl-1, brag-1, mcl-1, cIAP1, cIAP2, cIAP3, XIAP, synoviolin, MDM4, cullin 4a (CUL4A), cullin 7 (CUL7), HECT, UBA and WWE domain containing 1 (Huwe1; also termed Mule or ARF-BP1), synoviolin, SET and MYND domain containing 2 (Smyd2; also termed KMT3C), Proteasome activator complex subunit 3 (PMSE3), nuclear receptor subfamily 4, group A (NR4A1), protein phosphatase 1, magnesium-dependent, delta isoform (PM1D), topoisomerase I-binding protein (TOPORS), sirtuin (silent mating type information regulation 2 homolog) 2 (SIRT2), mediator of DNA-damage checkpoint 1 (MDC1) and survivin in the host cells.

Expression of multiple silencing factors such as shRNA molecules from a single vector can be accomplished in different ways. One way is to insert multiple expression cassettes into the vector, one for each RNAi molecule (e.g., Ter Brake et al., Mol. Ther. 14 (2006):883-892). Another way is to express an extended shRNA that can be processed by the RNAi machinery into multiple siRNA duplex molecules (e.g., Liu et al., Nucl. Acids Res. 35 (2007):5683-5693). Yet another way is to express multiple shRNA molecules from a single polycistronic transcript, e.g. derived from a natural miRNA cluster such as the miR-17-92 polycistron (Liu et al., Nucl. Acids Res. 36 (2008):2811-2824).

It is to be understood that the invention also includes replication competent adenoviruses according to the invention that additionally comprise one or more further modifications to change their characteristics, including but not limited to a change of tropism through pseudotyping or targeting, such as e.g. described by Krasnykh et al. (Mol. Ther. 1 (2000):391-405), Suzuki et al. (Clin. Cancer Res. 7(20012):120-126), Van Beusechem et al. (Gene Ther. 10 (2003):1982-1991), Schagen et al. (Mol. Ther. 13 (2006):997-1005) and Havenga et al. (J. Virol. 76 (2002):4612-4620). A preferred adenovirus according to the invention is a human adenovirus, preferably of serotype 5.

A preferred replication competent adenovirus is a conditionally replicating adenovirus (CRAd). As indicated hereinabove, a CRAd will only replicate in cells in which the particular conditions exist that are required for replication of the CRAd. CRAds are designed to meet the specific requirements for replication in a chosen (first) type of cell and not in other (second) types of cells. This property makes CRAds particularly useful for several embodiments of the present invention where the intend is to treat a disease by specific lytic replication of the recombinant adenovirus according to the invention in diseased cells in an animal or human body resulting in specific removal of said diseased cells from said body. CRAds have been developed to selectively replicate in and kill cancer cells. Such cancer-specific CRAds represent a novel and very promising class of anticancer agents (reviewed by Heise and Kirn, supra, Alemany et al., supra; Gomez-Navarro and Curiel, supra). The tumor-selective replication of this type of CRAds is achieved through either of two alternative strategies. In a first strategy, the expression of an essential early adenovirus gene is controlled by a tumor-specific promoter (e.g., Rodriguez et al., Cancer Res. 57 (1997):2559-2563; Hallenbeck et al., Hum. Gene Ther. 10 (1999):1721-1733; Tsukuda et al., Cancer Res. 62 (2002):3438-3447; Huang et al., Gene Ther. 10 (2003):1241-1247; Cuevas et al., Cancer Res. 63 (2003):6877-6884). The second strategy involves the introduction of mutations in viral genes to abrogate the interaction of the encoded RNA or protein products with cellular proteins, necessary to complete the viral life cycle in normal cells, but not in tumor cells (e.g., Bischoff et al., Science 274 (1996):373-376; Fueyo et al., Oncogene 19 (2000):2-12; Heise et al., Clin. Cancer Res. 6 (2000):4908-4914; Shen et al., J. Virol. 75(2001:4297-4307; Cascallo et al., Cancer Res. 63 (2003):5544-5550). During their replication in tumor cells CRAds destroy cancer cells by inducing lysis, a process that is further referred to as “oncolysis”. The restriction of CRAd replication to cancer or hyperproliferative cells dictates the safety of the agent, by preventing lysis of normal tissue cells. Currently, CRAd-based cancer treatments are already being evaluated in clinical trials (e.g., Nemunaitis et al., Cancer Res. 60 (2000):6359-6366; Khuri et al., Nature Med. 6 (2000):879-885; Habib et al., Hum. Gene Ther. 12 (2001):219-226).

A preferred conditionally replicating adenovirus according to the invention comprises a mutation in the ElA region encompassing at least a part of the CR2 domain of ElA, preferably a deletion encompassing amino acids 122 to 129 (LTCHEAGF) of ElA, providing conditional replication properties.

A replication competent adenovirus of the invention may further be modified to express one or more transgenes, such as e.g. a gene encoding a cytokine, a pro-apoptotic protein, an anti-angiogenic protein, a membrane fusogenic protein or a prodrug converting enzyme; or may carry mutations that increase its replication potential, such as e.g. retention of the E3 region (Suzuki et al., Clin. Cancer Res. 8 (2002):3348-3359) or deletion of the E1B-19K gene (Sauthoff et al. Hum. Gene Ther. 11 (2000):379-388), or that increase the replication selectivity for a certain type of cells, including but not limited to the modifications to make CRAds (supra), or that reduce the immunogenicity (i.e., their potency to induce an immune response when introduced into an animal body), such as e.g. retention of the E3B region (Wang et al., Nature Biotechnol. 21 (2003):1328-1335).

The at least one DNA sequence coding for a silencing factor functional in reducing expression of a target gene is preferably inserted into the viral genome as an expression cassette that includes the one or more expression control sequences that mediate expression of the DNA sequence in the host cell. The invention does not dictate the site of insertion of the at least one DNA sequence coding for a silencing factor functionally linked to regulatory DNA sequences in the genome of said replication competent adenovirus; said insertion may be at any location in said genome that does not inhibit replication of said replication competent adenovirus in said cell into which said replication competent adenovirus is introduced and where endogenous expression cassettes in said genome do not interfere with proper expression of said silencing factor. In non-limiting examples of the invention, said insertion is a replacement of the adenovirus E3-region or insertion between the E4 promoter and the right-hand ITR. DNA constructs to generate recombinant adenoviruses with insertions in the E3-region are known in the art, including but not limited to pBHG10 and pBHG11 (Bett et al., Proc. Natl. Acad. Sci. USA 91(1994):8802-8806), and insertions at other sites within the adenovirus genome can be made using standard molecular biology methods known in the art. In specific situations, it is preferred for proper expression of said open reading frame to shield said DNA sequence coding for a silencing factor functionally linked to regulatory DNA sequences from other regulatory DNA sequences present in said adenovirus genome by flanking said DNA sequence coding for a silencing factor functionally linked to regulatory DNA sequences by so-called insulator elements (Steinwaerder and Lieber, Gene Therapy 7 (2000):556-567). In another variation of the invention, said DNA sequence coding for a silencing factor is inserted in place of an adenovirus gene, where it is preferred that said adenovirus gene is expressed during the late phase of adenovirus replication, and where it is further preferred that said adenovirus gene is functionally linked to the endogenous major late promoter (MLP). Two or more expression cassettes may be inserted at the same of at different positions in the adenoviral genome.

The one or more expression control sequences preferably comprise a PolIII promoter, selected from the U6, H1, 7SK and tRNA(Val) promoters. Preferred expression control sequences comprise a U6 promoter. When more than one silencing factor is expressed, it is preferred that each silencing factor is driven by a different promoter, e.g. using a selection of the U6, H1, 7SK and tRNA(Val) promoters. In another variation of the invention, said more than one silencing factors are expressed from the virus genome on a single transcript encoding an extended short hairpin. In another variation of the invention, said more than one silencing factors are expressed from the virus genome on a single polycistronic transcript. In this variation, it is preferred that said expression is driven by a PolII promoter.

In one variation of the invention, said host cell is a cell that is being cultured in vitro. In another variation of the invention, said host cell is a cell in an animal body, where it is preferred that said animal body is a human body.

A preferred host cell in which said replication competent adenovirus comprising in the genome thereof, one or more DNA sequences coding for a silencing factor that is functional in reducing expression of synoviolin in said host cell, operably linked to one or more expression control sequences, functional in the said host cell, is a cell expressing synoviolin. Preferably said synoviolin expressing cell is a cancer cell. The amount of silencing brought about by the one or more silencing factors is preferably sufficient to decrease the amount of synoviolin to a level that allows said effective lytic propagation capacity of the replication competent adenovirus of the invention in said cell. The invention contemplates that knowledge of synoviolin expression in cells is useful to stratify between cells that are more susceptible and cells that are less susceptible to lytic replication of replication competent adenoviruses. The invention furthermore provides that knowledge of synoviolin expression in cells is useful to differentiate between cells in which the replication competent adenoviruses suppressing synoviolin according to the invention exert more effective lytic propagation capacity compared to replication competent adenoviruses that are not suppressing synoviolin according to the invention; and cells in which the replication competent adenoviruses suppressing synoviolin according to the invention do not exert more effective lytic propagation capacity compared to replication competent adenoviruses that are not suppressing synoviolin according to the invention. In preferred embodiments, said cells are cells with a defect in a cell death pathway, in particular cancer cells and/or said cells are human cells. In variations of the invention, said knowledge is obtained at the synoviolin mRNA level; at the synoviolin protein level; or at the functional synoviolin protein activity level, in particular the activity of synoviolin to inhibit p53 activity in cells. Methods to determine synoviolin expression in cells are known in the art. Non-limiting examples of said methods include quantitative reverse transcription polymerase chain reaction (qRT-PCR) or hybridization to DNA or oligonucleotide probes, such as e.g. in microarray analysis to determine expression at the mRNA level; and ELISA, Western analysis, immunocytochemical staining and immunohistochemical staining to determine expression at the protein level. A non-limiting example of a method to determine the impact of synoviolin expression on p53 activity in cells is given in Example 15. In a variation of said method, the p53 reporter plasmid is transfected into the cell transiently in stead of stably and a transfection efficiency control is included (e.g., van Beusechem et al., Cancer Res. 62 (2002):6165-6171; SABiosciences Cignal p53 reporter kit CCS-004L).

In one preferred embodiment of the invention, said host cell expresses a functional p53 tumor suppressor gene, wherein it is further preferred that the activity of the p53 protein encoded by said functional p53 gene is inhibited by synoviolin. In this embodiment, the amount of synoviolin silencing brought about by said one or more silencing factors should be sufficient to increase the activity of the p53 protein to a level that allows said effective lytic propagation capacity of the replication competent adenovirus of the invention in said cell. Said cell is preferably a human cell, preferably a cancer or tumor cell. Preferred cancer cells are cancer cells with an elevated level of synoviolin expression. According to present knowledge, elevated synoviolin expression is common in cancer cells selected from breast cancer cells, lung cancer cells, in particular non small cell lung cancer cells, head and neck cancer cells, liver cancer cells, melanoma cells, colorectal cancer cells, urothelial cancer cells and thyroid cancer cells. Most preferred cancer cells are lung cancer cells, preferably non small cell lung cancer cells. A preferred cancer cell line is the A549 non small cell lung cancer cell line.

In a further embodiment, the invention provides an adenovirus according to the invention for use in a medicament for the treatment of cancer, wherein the cancer is preferably selected from lung cancer, breast cancer, head and neck cancer, liver cancer, melanoma, thyroid cancer, colorectal cancer and urothelial cancer, more preferred lung cancer cells such as non small cell lung cancer cells.

In a further embodiment, the invention provides a kit of parts comprising at least one DNA sequence coding for a silencing factor functional in reducing expression of synoviolin in a cancer cell and a replication competent virus, preferably an adenovirus. Further provided is a kit of parts according to the invention for use in a medicament for the treatment of cancer, preferably a carcinoma, wherein the cancer is preferably selected from lung cancer, breast cancer, head and neck cancer, liver cancer, melanoma, thyroid cancer, colorectal cancer and urothelial cancer.

The invention also provides a method of lysing a cancer cell comprising the step of providing the cancer cell with a virus according to the invention, thereby inducing lysis of the cancer cell and release of virus progeny from the cancer cell. Alternatively, or in addition, the invention provides a method of lysing a cancer cell comprising the step of providing the cancer cell with the kit of parts according to the invention, thereby inducing lysis of the cancer cell and release of virus progeny from the cancer cell.

Also provided by the present invention is a method for treatment of a subject suffering from a cancer, whereby the cancer is selected from lung cancer, breast cancer, head and neck cancer, liver cancer, melanoma, thyroid cancer, colorectal cancer and urothelial cancer, the method comprising the step of administering to the said subject an effective amount of the replication competent virus according to the invention. Alternatively, or in addition, the invention provides a method for treatment of a subject suffering from a cancer, whereby the cancer is selected from lung cancer, breast cancer, head and neck cancer, liver cancer, melanoma, thyroid cancer, colorectal cancer and urothelial cancer, the method comprising the step of administering to the subject an effective amount of the kit of parts according to the invention.

In another preferred embodiment of the invention, a host cell is a cell with a defect in a cell death pathway, such as a host cell that is hampered in the p53-dependent cell death pathway. The viral genome of a replication competent virus according to the invention may therefore, in addition to the at least one DNA sequence coding for a silencing factor functional in reducing expression of a target gene in the said host cells and the one or more expression control sequences that mediate expression of the DNA sequence in the host cell, comprise the coding sequence of at least one restoring factor functional in restoring the p53-dependent apoptosis pathway in said host cells, operably linked to one or more expression control sequences, functional in the said host cells. Said restoring factor preferably is selected from the pro-apoptotic death genes of the bcl-2 family, such as bax, bak, bad, bid, bik, bim, bok, blk, hrk, puma, noxa and bcl-xS (Miyashita and Reed, Cell 80 (1995):293-299; Han et al., Genes Dev. 10 (1996):461-477; Zoernig et al., Biochim. Biophys. Acta 1551 (2001):F1-F37), and/or p53, or a functional part or derivative thereof.

The invention further provides the use of an inhibitor of synoviolin expression and/or an inhibitor of synoviolin activity in a synoviolin expressing cell for enhancing replication of an adenovirus in said cell. The invention further provides the use of an inhibitor of synoviolin expression and/or an inhibitor of synoviolin activity in a synoviolin expressing cell for enhancing lysis of a cancer cell. Preferably said adenovirus is a replication competent adenovirus. Preferably said replication competent adenovirus is a conditionally replicating adenovirus. In a preferred embodiment said inhibitor of synoviolin expression comprises an antisense RNA specific for and capable of hybridizing to synoviolin RNA. Said antisense RNA is preferably a member of an RNAi molecule. In a preferred embodiment said inhibitor of synoviolin activity comprises an intracellular antibody specific for synoviolin. In a preferred embodiment said antibody is specific for the site with which synoviolin binds p53 in a cell. In other preferred embodiments said inhibitor comprises a dominant-negative protein, a small inhibitor molecule, an RNA antisense nucleic acid, an RNA ribozyme, a recombinant zinc finger protein and/or an RNA interference molecule In a preferred embodiment said adenovirus comprises a coding region coding for said inhibitor of synoviolin expression and/or an inhibitor of synoviolin activity. The coding region is preferably operably linked with suitable transcription signals and suitable translation signals such as a suitable promoter, a suitable polyA and/or a suitable transcription termination signal as mentioned elsewhere herein. An inhibitor of synoviolin expression in a cell typically also inhibits synoviolin activity in the cell. An inhibitor of synoviolin can but does not have to inhibit synoviolin expression in the cell.

The invention further provides a replication competent adenovirus comprising an expression cassette for an inhibitor of synoviolin expression and/or an inhibitor of synoviolin activity in a host cell. Said expression cassette preferably codes for a synoviolin antisense RNA. The synoviolin antisense RNA is preferably specific for and capable of hybridizing with synoviolin RNA. Preferably, said expression cassette comprises a transcription unit for a synoviolin specific RNAi molecule. Said RNAi molecule is preferably a shRNA coding for both a synoviolin antisense RNA and the corresponding sense strand connected via a loop sequence. The shRNA can be encoded by a larger RNA from which it is subsequently cleaved upon expression of the larger RNA in the cell. Such a larger precursor RNA is preferably derived from a pri-miRNA.

The invention further provides a method for lysing a synoviolin expressing host cell, preferably a synoviolin expressing cancer cell, comprising providing said host cell with a replication competent adenovirus that comprises an expression cassette for an inhibitor of synoviolin expression and/or an inhibitor of synoviolin activity in a host cell.

The invention further provides a replication competent adenovirus comprising expression cassettes for two or more RNAi molecules specific for two or more p53 antagonists and/or inhibitors of the p53 pathway or a combination thereof in a cell. It has been found that a cancer cell can contain more than one p53 antagonists and/or inhibitors of the p53 pathway. Such cells are more effectively lysed when they are provided with RNAi molecules against at least two of those p53 antagonists and/or inhibitors of the p53 pathway.

Hereinafter, the invention will be further exemplified in examples and figures. The several examples show a number of ways to provide said replication competent adenoviruses, formulations, methods, compositions, and uses according to the invention. It is to be clearly understood that the description is not meant to in any way limit the scope of the invention as was explained in general terms above. Skilled artisans will be able to transfer the teachings of the present invention to other replication competent adenoviruses, silencing factors, formulations, methods, compositions, and uses without departing from the present invention.

### EXAMPLES

### Example 1

### Construction of Adenovirus Shuttle Vectors Carrying a Gateway Recombination Destination Cassette

To construct a shuttle vector carrying a Gateway recombination destination cassette between the adenovirus E4 region and the right-hand ITR, the construct pEndK/SpeI (generously provided by Dr. R. Alemany, Institut Catala d'Oncologia, Barcelona, Spain) was used. pEndK/SpeI was made by first digesting pTG3602 (Chartier et al., J. Virol, 70 (1996):4805-4810) with KpnI and religating the vector fragment comprising Ad5 map units 0-7 and 93-100 to create pEndK. Next, a unique SpeI site was introduced into pEndK by changing Ad5 nucleotide 35813 from A to T by site directed mutagenesis to create pEndK/SpeI. PEndK/SpeI carries PacI restriction sites flanking the two Ad5 ITRs. pEndK/SpeI was made compatible with the Gateway system by ligating the Gateway destination cassette rfa (Gateway Vector Conversion System; Invitrogen, Carlsbad, Calif.) as a blunt fragment into the SpeI site (filled in with Klenow polymerase). Plasmids were selected that contained the Gateway destination cassette with the coding sequence of the ccdB gene on the adenovirus R or L strand and were designated pEndK/DEST-R and pEndK/DEST-L, respectively.

To construct a shuttle vector carrying a Gateway recombination destination cassette in place of the adenovirus E3 region, the GATEWAY destination cassette rfa (from the Gateway Vector Conversion System) was first cloned into pBluescript SK(−)(Stratagene) digested with EcoRV to obtain pBSK-DEST. From this template, the DEST cassette was PCR amplified using primers 5′-GAGGTCGACGCGATCGATAAGCTTGATATC-3′ (SEQ ID NO 1) and 5′-TAGAACTAGTCGATCGCCCGGGCTGCAG-3′ (SEQ ID NO 2) with overhanging PvuI sites and digested with PvuI. This fragment was ligated in pBHG11 (Microbix) digested with PacI to obtain pBHG11-DEST_R.

To construct a shuttle vector carrying the full-length genome of a CRAd with a Gateway recombination destination cassette in place of the adenovirus E3 region, first pEndK/SpeI (supra) was digested with EcoRV and the EcoRV-fragment comprising the fiber gene from pBHG11 was inserted to create pEndK-Fiber. Next, the HpaI-fragment containing DEST_R from pBHG11-DEST_R was inserted into HpaI-digested pEndK-Fiber to create pEndK-Fiber_DEST_R. Finally, the Fiber_DEST_R containing SpeI fragment from pEndK-Fiber_DEST_R was inserted into pAdΔ24E3 (see example 7) digested with SpeI to replace the E3 region and fiber gene with the DEST_R_Fiber fragment from pEndK-Fiber_DEST_R. The resulting plasmid is pAdΔ24-DEST_R.

### Example 2

### General Method to Construct Plasmids with an shRNA Expression Cassette that can be Transported into an Adenovirus Shuttle Vector According to Example 1 by Gateway Recombination

The plasmid pSHAG-1 (Paddison et al., Genes Dev. 16 (2002) 948-958; generously provided by Dr. G. J. Hannon, Cold Spring Harbor Laboratory, NY) is used as entry clone for the GATEWAY system (Invitrogen, Carlsbad, Calif.). pSHAG-1 contains a U6 promoter-driven expression cassette flanked by the attL1 and attL2 recombination sites such that the expression cassette can be transported into destination plasmid vectors including pEndK/DEST-R, pEndK/DEST-L, pBHG11-DEST_R and pAdΔ24-DEST_R of example 1 using the Gateway system. shRNA-encoding sequences can be introduced by ligation of pSHAG-1 digested with BseRI and BamHI with two annealed synthetic oligonucleotides with compatible overhanging DNA sequences. The first of the two oligonucleotides should be designed to contain in the 5′ to 3′ order: a first stretch of at least 19 and preferably no more than 29 nucleotides complementary to the target mRNA (i.e., antisense), a loop sequence, a second stretch of nucleotides of the same length and of reverse complementary sequence to the first stretch of nucleotides, and a stretch of at least 4 thymidines. The second oligonucleotide should be reverse complementary to the first oligonucleotide. Furthermore, when annealed the then double-stranded oligonucleotides should form overhanging sites compatible with BseRI and BamHI restriction sites. Depending on the choice of the sequence of 19 to 29 nucleotides a useful shRNA for the invention directed against a target of choice can be made. For example, useful oligonucleotide sequences to target firefly luciferase are: Oligonucleotide 1: 5′-GATTCCAATTCAGCGGGAGCCACCTGATgaagcttgATCGGGTGGCTCTCGCTGAGTTGGAA TCCATTTTTT-3′ (SEQ ID NO 3); and Oligonucleotide 2: 5′-GATCAAAAAATGGATTCCAACTCAGCGAGAGCCACCCGATcaagcttcATCAGGTGGCTCCC GCTGAATTGGAATCCG-3′ (SEQ ID NO 4), where the lower case letters represent the loop sequence. Annealing of oligonucleotides 1 and 2 followed by ligation into pSHAG-1 digested with BseRI and BamHI results in the formation of pSHAG-shRNA. In the case of the luciferase-specific shRNA example this results in pSHAG-Ff1 that encodes a shRNA homologous to nucleotide 1340 to 1368 of the coding sequence of the firefly luciferase gene.

### Example 3

### General Methods to Construct Adenovirus Shuttle Vectors Carrying an shRNA Expression Cassette Using the Plasmids from Examples 1 and 2

To construct an adenoviral shuttle vector carrying an shRNA-expression cassette inserted between the E4 region and the right-hand ITR, the shRNA expression cassette is transferred from the pSHAG-shRNA construct of example 2 to the pEndK/DEST-R or pEndK/DEST-L plasmid of example 1 via an in vitro GATEWAY LR recombination reaction using the GATEWAY LR Clonase enzyme mix (Invitrogen) according to manufacturer's protocol. This results in pEndK/shRNA-R or pEndK/shRNA-L. For example, pSHAG-Ff1 is recombined with pEndK/DEST-R or pEndK/DEST-L creating pEndK-Ff1-R or pEndK-Ff1-L, respectively.

To construct an adenoviral shuttle vector carrying an shRNA-expression cassette inserted in place of the E3 region, the shRNA expression cassette is transferred from the pSHAG-shRNA construct of example 2 to the pBHG11-DEST_R plasmid of example 1 via the same in vitro GATEWAY LR recombination reaction to create pBHG11-shRNA. To construct a plasmid carrying the full-length genome of an AdΔ24-type CRAd (Fueyo et al., Oncogene 19 (2000):2-12) with an shRNA-expression cassette inserted in place of the adenovirus E3 region, the shRNA expression cassette is transferred from the pSHAG-shRNA construct of example 2 to the pAdΔ24-DEST_R plasmid of example 1 via the same in vitro GATEWAY LR recombination reaction to create pAdΔ24-shRNA.

### Example 4

### General Methods to Construct Replication Competent Adenoviruses Expressing shRNA Molecules Using a Plasmid According to Example 3

The plasmids pEndK/shRNA-R and pEndK/shRNA-L can be linearized with KpnI and/or EcoRV. This separates the Ad5 map units 0-7 from Ad5 map units 93-100 with the inserted shRNA expression cassette. These linearized molecules can be recombined in bacteria, for example in E. coli BJ5183, with full-length replication competent adenovirus DNA. Said full-length replication competent adenovirus DNA can be isolated from adenovirus particles or, alternatively can be released by digestion from a plasmid carrying a full-length replication competent adenovirus DNA insert. Double homologous recombination then creates a plasmid with a full-length replication competent adenovirus genome insert, in which the shRNA expression cassette is inserted between the E4 region and the right-hand ITR. It should be noted that any full-length replication competent adenovirus can be used to insert shRNA expression cassettes according to this method, including recombinant adenoviruses with additional modifications, such as e.g. enhanced tumor-selectivity or oncolytic potential, a changed tropism or transgene insertion. It is preferred, however, that said full-length replication competent adenovirus does not include a PacI restriction site in its genome. The complete replication competent adenovirus genome with inserted shRNA expression cassette is subsequently released from the plasmid by PacI digestion. This DNA is transfected into human cells using, e.g., lipofectamine reagent. The resulting recombinant replication competent adenovirus according to the invention is isolated and further propagated and purified according to standard cell culture and virology methods known in the art.

pBHG11-shRNA plasmids are transfected into human cells together with pXC1 (Microbix Biosystems) or pXC1-derived plasmids with modifications of choice, e.g., in the E1 region to create CRAds including but not limited to the Δ24-mutation (infra), to allow homologous recombination reconstituting a complete replication competent adenovirus genome with the shRNA expression cassette inserted in place of the E3 region. This virus can then be isolated, propagated, purified and used according to methods known in the art. The pAdΔ24-shRNA plasmid can be digested with PacI and transfected into human cells to isolate a Δ24-type CRAd with the shRNA expression cassette inserted in place of the E3 region. This virus can then also be isolated, propagated, purified and used according to methods known in the art.

### Example 5

### Construction of Firefly Luciferase-Silencing Conditionally Replicating Adenoviruses Ad5-Δ24E3-Ff1-R and Ad5-Δ24E3-Ff1-L

The Ad5 derived conditionally replicating adenovirus (CRAd) Ad5-Δ24E3 carrying a 24-bp deletion in the pRb-binding CR2 domain in E1A (Suzuki et al., Clin. Cancer Res. 8 (2002): 3348-3359) was used as backbone to construct CRAds expressing shRNA molecules against firefly luciferase. According to the general method described in example 4, homologous recombination was performed in E. coli BJ5183 between full-length Ad5-Δ24E3 viral DNA and KpnI-digested pEndK-Ff1-R or pEndK-Ff1-L (see example 3) to form plasmids pAdΔ24E3-Ff1-R and pAdΔ24E3-Ff1-L, respectively. These plasmids were digested with PacI to release the full-length adenoviral DNA with Ff1 shRNA expression cassette insert from the plasmid backbone and were transfected into human 293 cells (Graham et al., J. Gen. Virol. 36 (1977):59-74). Ad5-Δ24E3.Ff1-R and Ad5-Δ24E3.Ff1-L CRAds were harvested and further propagated on A549 cells (obtained from the ATCC). The E1Δ24 deletion and the U6-Ff1 insertion and orientation were confirmed by PCR on the final products and functional PFU titers were determined by limiting-dilution plaque titration on 293 cells according to standard techniques.

### Example 6

### Specific Silencing of Firefly Luciferase by Conditionally Replicating Adenoviruses Ad5-Δ24E3-Ff1-R and Ad5-Δ24E3-Ff1-L in Human Cancer Cells

In order to accurately quantify silencing efficiency and to adequately compensate for experimental variation, we employed the reporter plasmid phAR-FF-RL that expresses firefly and Renilla luciferase genes from the bidirectional hAR promoter (Barski et al., Biotechniques 36(2004): 382-4, 386, 388), thus allowing normalization of firefly luciferase silencing relative to Renilla luciferase expression. To construct phAR-FF-RL, the hAR promoter (nt −124 to +29 relative to the transcription start site, Genbank accession number AF112482) was obtained by PCR using subcloned genomic DNA as template and primers 5′-CCAGAAGAGCTCGCAACGTGGCATCTGCTA-3′ (SEQ ID NO 5) and 5′-GTTTGGAGAGCTCCTGGGCACAATGAGGC-3′ (SEQ ID NO 6), creating flanking SacI sites (underlined). The PCR product was inserted in the SacI site of pGL3-basic (Promega, Madison, Wis.) with the 3′-end of the promoter towards the firefly luciferase gene, creating phAR-FF. Next, the Renilla luciferase cDNA was obtained by PCR using pRL-TK (Promega) as template and primers 5′-ACAACGGTACCGAACTTAAGCTGCAG-3′ (SEQ ID NO 7) and 5′-CCGAAAGGTACCACCTGGATCCTTATC-3′ (SEQ ID NO 8), creating flanking KpnI sites (underlined) and inserted into the KpnI site of phAR-FF in an orientation opposite to that of the firefly luciferase gene, such that the 5′-end of the hAR promoter faces the 5′ side of the Renilla luciferase gene.

Human non small cell lung carcinoma A549 cells, breast carcinoma MCF-7 cells and cervix carcinoma HeLa cells were obtained from the American Type Culture Collection (Manassas, Va.). Osteosarcoma SaOs-2 cells were a kind gift of Dr. F. van Valen (Westfalische Wilhelms-Universität, Munster, Germany). Cells were maintained in F12-supplemented DMEM with 10% fetal calf serum, 50 IU ml⁻¹ penicillin and 50 μg ml⁻¹ streptomycin (Life Technologies, Inc., Paisley, United Kingdom). The human cancer cells lines were plated at 50-70% confluence in 24-well plates and transfected with 50 ng phAR-FF-RL and 250 ng irrelevant plasmid pBluescript SK(−) carrier DNA (Stratagene, La Jolla, Calif.) using Lipofectamine Plus (Invitrogen) according to the manufacturer's protocol. Infections with control CRAd Ad5-Δ24E3 or with silencing CRAds Ad5-Δ24E3.Ff1-R or Ad5-Δ24E3.Ff1-L (supra) were performed at a multiplicity of infection of 500 PFU/cell for 2 hours at 37° C., immediately followed by transfection. Activities of firefly and Renilla luciferase were determined 30 h post infection using the Dual-Luciferase Reporter Assay System (Promega) according to the manufacturer's protocol.

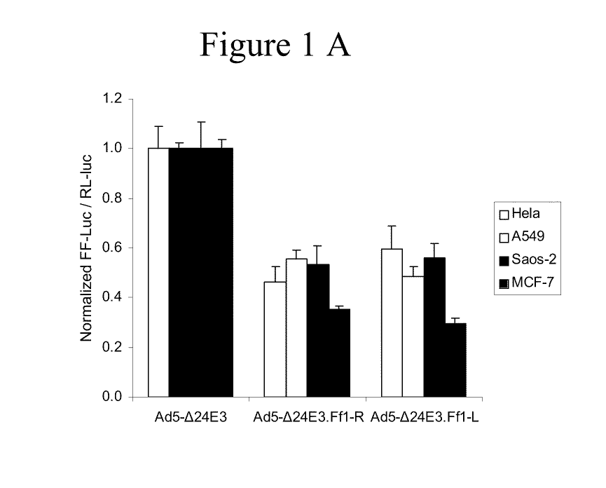

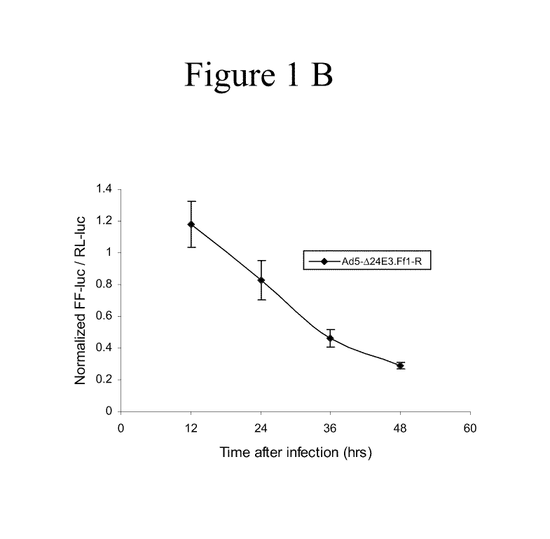

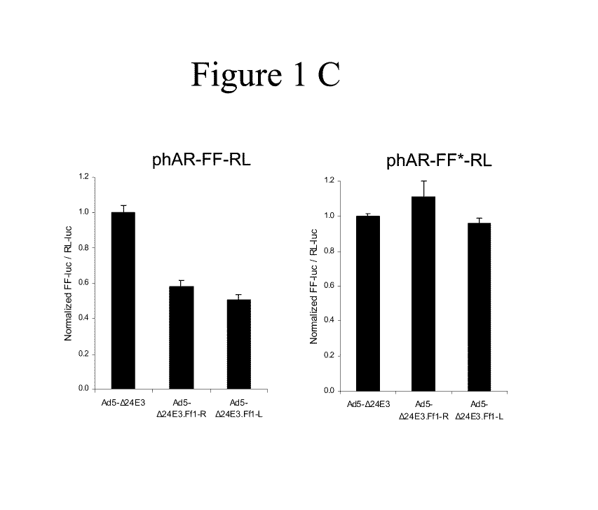

On different cell lines, Ad5-Δ24E3.Ff1-R and Ad5-Δ24E3.Ff1-L each suppressed the normalized firefly luciferase expression to approximately 60% to 30% of the expression level observed after infection with the parental control CRAd Ad5-Δ24E3 (FIG. 1A). This demonstrated that shRNAs expressed from CRAds are able to suppress the expression of a target gene in host cells infected with said CRAds.

Because the shRNAs are encoded by replicating adenoviruses it was assumed likely that as a consequence of virus genome replication their expression would increase in time. Pilot experiments using Ad5Δ24-CMV-luc (example 10) revealed that in A549 an exponential increase in transgene expression started at 20 hours post-infection to reach a plateau at 32 hours. Assuming that the expression of the shRNAs driven by the U6-promoter follows a similar expression profile we anticipated that the silencing effect induced by the shRNAs would increase during the replicative cycle. To test this, we performed a time course experiment measuring the silencing by Ad5-Δ24E3.Ff1-R in A549 cells at 12, 24, 36 and 48 hours post-infection. This experiment showed that Ad5-Δ24E3.Ff1-R progressively suppressed firefly luciferase expression in A549 cells over the first two days post-infection (FIG. 1B). At 48 hours post-infection, when CPE became apparent, Ad5-Δ24E3.U6-Ff1-R had silenced firefly luciferase down to approximately 30% of the Ad5-Δ24E3 control.

Since the firefly luciferase expression values were normalized using Renilla luciferase expression as an internal control and effects of shRNA-expressing CRAds were compared to expression after infection with the Ad5-Δ24E3 parental virus, it is unlikely that the observed suppression of firefly luciferase expression was due to nonspecific effects caused by viral replication. However, to formally exclude this possibility, we investigated silencing of a mutant firefly luciferase. To this end, we introduced six silent point mutations in the shRNA target sequence in the firefly luciferase gene encoded by phAR-FF-RL. We made control reporter plasmid phAR-FF*-RL that is similar to phAR-FF-RL but contains silent mutations in the target recognition site of firefly luciferase changing the recognition sequence into: 5′-ACCAGGTTGCCCCTGCTGAGTTGGAATCG-3′ (SEQ ID NO 9) (mutations are underlined). The mutations were first introduced in pGL2 (Promega) were the target recognition site is fortuitously flanked by EcoRV and ClaI restriction sites allowing the use of a small linker to introduce the mutations. For this purpose the oligonucleotides 5′-ACCAGGTTGCCCCTGCTGAGTTGGAAT-3′ (SEQ ID NO 10) and 5′-CGATTCCAACTCAGCAGGGGCAACCTGGT-3′ (SEQ ID NO 11) were annealed and treated with kinase. This linker was ligated into pGL2 digested with EcoRV and ClaI, replacing the unmodified target sequence. From this vector, the 765 bp SphI-SgrAI fragment containing the firefly luciferase sequence with the mutated target site was ligated into phAR-FF-RL replacing the corresponding SphI-SgrAI region. As expected, the mutations introduced in phAR-FF*-RL did not influence the activity of the firefly luciferase protein but abolished Ff1 shRNA-mediated silencing, as was confirmed by co-transfection of the mutated reporter plasmid (phAR-FF*-RL) with pSHAG-Ff1. A549 cells were infected with Ad5-Δ24E3, Ad5-Δ24E3.U6-Ff1-R, or Ad5-Δ24E3.U6-Ff1-L, followed by transfection with the reporter plasmid phAR-FF-RL or the mutated reporter plasmid phAR-FF*-RL. FIG. 1C shows that both silencing CRAds suppressed firefly luciferase expression 30 hours post-infection as before to approximately 50%, but did not change expression of mutant firefly luciferase. This confirmed that the observed silencing of firefly luciferase expression was dependent on the shRNA target sequence and is brought about via the mammalian RNAi pathway.

### Example 7

### Construction of Conditionally Replicating Adenoviruses that Silence a p53 Antagonist

A derivative of Ad5-Δ24E3 (Suzuki et al., Clin. Cancer Res. 8 (2002):3348-3359) expressing an shRNA directed against HPV18-E6 driven by the PolIII H1 promoter was made the following way. First, Ad5-Δ24E3 linear dsDNA was isolated from virions and recombined with linearized pEndK/Spe (supra) in BJ5183 bacteria to obtain plasmid clone pAdΔ24E3, from which full length AdΔ24E3 DNA was released by PacI digestion. Next, a synthetic double-stranded oligonucleotide consisting of a 19 nt sequence from HPV18 E6 (nucleotides 385-403, numbering according to Cole and Danos, J. Mol. Biol. 193 (1987):599-608), followed by a 9-nt loop linker (lower case letters) and the reverse complement of the 19-nt HPV18 E6 sequence, was inserted into the plasmid pSUPER (Brummelkamp et al., Science 296 (2002):550-553) to create pSUPER-18E6. To this end, the oligonucleotides FP_super18E6 (5′-gatccccCTAACACTGGGTTATACAAttcaagagaTTGTATAACCCAGTGTTAGtttttgga aa-3′) (SEQ ID NO 12) and RP_super18E6 (5′-agcttttccaaaaaCTAACACTGGGTTATACAAtctcttgaaTTGTATAACCCAGTGTTAGg gg-3′) (SEQ ID NO 13) were annealed and ligated into BglII/HindIII digested pSUPER. pSUPER-18E6 was then digested with HindIII, overhanging ends filled with Klenow polymerase, and relegated to create an NheI site. Subsequently, it was digested with SpeI and NheI to release the H1_{—}18E6 fragment, which was then inserted into SpeI-digested pEndK/SpeI, creating plasmid pEndK.H1_{—}18E6. Functional silencing of HPV-18 E6 resulting in a decreased inhibition of p53 activity in HPV-18 transformed human cancer cells was confirmed by transfecting pEndK.H1_{—}18E6 together with p53-specific reporter plasmid PG13-Luc (el-Deiry et al., Cell 75 (1993):817-825) into HeLa cervical carcinoma cells and measuring luciferase expression. This revealed significantly higher luciferase activity than was measured following control transfection of pEndK/SpeI with PG13-luc or following transfection of PG13-Luc alone. Thus, the shRNA expressed by pEndK.H1_{—}18E6 silenced a p53 antagonist. Next, pEndK.H1_{—}18E6 was linearized by digestion with KpnI and EcoRV and recombined with the PacI-linearized Ad5-Δ24E3 DNA in E. coli BJ5183 cells. This created pAdΔ24.H1_{—}18E6. PacI-linearized pAdΔ24.H1_{—}18E6 was transfected onto 911 cells (Fallaux et al., Hum. Gene Ther. 7 (1996):215-222) and AdΔ24.H1_{—}18E6 virus was isolated and further propagated on A549 cells.

Several CRAds with the Δ24-mutation in the E1 region (Fueyo et al., Oncogene 19 (2000):2-12) and each expressing a different shRNA specific for the p53 antagonists polo-like kinase-1 (plk-1) and parc were made the following way. For both target genes, three different silencing constructs were designed that target different sequences of the target mRNA. For each silencing construct, a set of two oligonucleotides was synthesized, the sequences whereof are given below. These oligonucleotides were allowed to anneal and were inserted into pSHAG-1 digested with BseRI and BamHI according to example 2. The shRNA expression cassettes from the resulting pSHAG-shRNA constructs were transferred into pAdΔ24-DEST_R (example 1) via an LR GATEWAY in vitro recombination reaction according to example 3. Full-length clones were digested with PacI and transfected in 911 cells to obtain shRNA-expressing replication competent adenovirus, which was further propagated on A549 cells.

The oligonucleotide sets used were: for plk-1; set-A: 5′-GGCGGCTTTGCCAAGTGCTTCTCGAGAAGCACTTGGCAAAGCCGCCCTTTTT-3′ (SEQ ID NO 14) and 5′-GATCAAAAAGGGCGGCTTTGCCAAGTGCTTCTCGAGAAGCACTTGGCAAAGCCGCCCG-3′ (SEQ ID NO 15); set-B: 5′-GCCGCCTCCCTCATCCAGAACTCGAGTTCTGGATGAGGGAGGCGGCCTTTTT-3′ (SEQ ID NO 16) and 5′-GATCAAAAAGGCCGCCTCCCTCATCCAGAACTCGAGTTCTGGATGAGGGAGGCGGCCG-3′(SEQ ID NO 17); and set-C: 5′-ATGAAGAAGATCACCCTCCTTACTCGAGTAAGGAGGGTGATCTTCTTCATCTTTTT-3′ (SEQ ID NO 18) and 5′-GATCAAAAAGATGAAGAAGATCACCCTCCTTACTCGAGTAAGGAGGGTGATCTTCTTCATCG-3′ (SEQ ID NO 19); and for parc; set-A: 5′-GAAGCTTTCCTCGAGATCCACTTCCTGTCATGGATCTCGAGGAAAGCTTCCTTTTT-3′ (SEQ ID NO 20) and 5′-GATCAAAAAGGAAGCTTTCCTCGAGATCCATGACAGGAAGTGGATCTCGAGGAAAGCTTCCG-3′ (SEQ ID NO 21); set-B: 5′-GCATCGAGCAGCACATGGATCTTCCTGTCAATCCATGTGCTGCTCGATGCCTTTTT-3′(SEQ ID NO 22) and 5′-GATCAAAAAGGCATCGAGCAGCACATGGATTGACAGGAAGATCCATGTGCTGCTCGATGCCG-3′ (SEQ ID NO 23); and set-C: 5′-CTCGCCAGGAGAAGCGGTTTCTTCCTGTCAAAACCGCTTCTCCTGGCGAGCTTTTT-3′ (SEQ ID NO 24) and 5′-GATCAAAAAGCTCGCCAGGAGAAGCGGTTTTGACAGGAAGAAACCGCTTCTCCTGGCGAGCG-3′ (SEQ ID NO 25).

### Example 8

### Construction of a Conditionally Replicating Adenovirus that Silences a p53 Antagonist and Additionally Expresses a Functional Factor of the p53 Dependent Apoptosis Pathway

A derivative of Ad5-Δ24E3 (Suzuki et al., Clin. Cancer Res. 8 (2002):3348-3359) expressing an shRNA directed against HPV18-E6 driven by the PolIII H1 promoter and additionally expressing human p53 was made the following way. First, the plasmid pEndK/p53 was made. To this end, a derivative of pBluescript SK(−) (Stratagene, La Jolla, Calif.) lacking the EcoRV site was made by digesting pBluescript SK(−) with SmaI and EcoRV followed by self-ligation. This vector was digested with KpnI and SalI and the KpnI/SalI fragment containing the SV40 promoter-driven human p53 expression cassette from pABS.4-p53 (Van Beusechem et al., Cancer Res. 62 (2002):6165-6171) was inserted to create pBSK-p53. Subsequently, the KpnI site in pBSK-p53 was changed into a SpeI site. This was done by digesting pBSK-p53 with KpnI and inserting a synthetic double-stranded oligonucleotide made by annealing oligo 5′-TCAGGACTAGTGGAATGTAC-3′ (SEQ ID NO 26) and oligo 5′-ATTCCACTAGTCCTGAGTAC-3′ (SEQ ID NO 27). The 2.6 kb SV40-p53 fragment was then released by SpeI digestion and inserted into SpeI-digested pEndK/Spe (supra). A clone with an insert in the orientation that places the SV40-p53 cassette on the adenovirus L-strand was isolated and designated pEndK/p53. Expression of functional p53 from pEndK/p53 was confirmed by transfecting pEndK/p53 or control construct pEndK/SpeI together with p53-specific reporter plasmid PG13-Luc or with negative control plasmid MG15-Luc (el-Deiry et al., Cell 75 (1993):817-825) into p53-null SaOs-2 osteosarcoma cells and measuring luciferase expression the next day. Transfection of pEndK/p53 resulted in a p53-specific PG-13/MG-15 ratio of 63, whereas the PG-13/MG-15 ratio after transfection of empty pEndK/SpeI vector was only 0.7. Next, pEndK/p53 was digested with ClaI and filled in with Klenow polymerase following which the HincII/SmaI fragment containing the H1-18E6 fragment from pSUPER-18E6 (supra) was inserted, creating pEndK/p53.H1_{—}18E6. Finally, pEndK/p53.H1_{—}18E6 was linearized by digestion with KpnI and EcoRV and recombined with PacI-linearized Ad5-Δ24E3 DNA (supra) in E. coli BJ5183 cells to create pAdΔ24.p53(L).H1_{—}18E6. This vector was PacI-linearized and transfected onto 911 packaging cells. AdΔ24.p53(L).H1_{—}18E6 virus was isolated and further propagated on A549 cells.

### Example 9

### Construction of a Conditionally Replicating Adenovirus that Silences a p53 Pathway Inhibitor

CRAds with the Δ24-mutation in the E1 region (Fueyo et al., Oncogene 19 (2000):2-12) and each expressing a different shRNA specific for bcl-2 were made using the same procedure described in example 7 to make CRAds with shRNAs for plk-1 or parc. Only different sets of oligonucleotides were used, specific for target sequences on the bcl-2 mRNA, i.e., set-A: 5′-CTGCACCTGACGCCCTTCACCTTCCTGTCAGTGAAGGGCGTCAGGTGCAGCTTTTT-3′(SEQ ID NO 28) and 5′-GATCAAAAAGCTGCACCTGACGCCCTTCACTGACAGGAAGGTGAAGGGCGTCAGGTGCAGCG-3′ (SEQ ID NO 29); set-B: 5′-GGAGGATTGTGGCCTTCTTTCTTCCTGTCAAAAGAAGGCCACAATCCTCCCTTTTT-3′ (SEQ ID NO 30) and 5′-GATCAAAAAGGGAGGATTGTGGCCTTCTTTTGACAGGAAGAAAGAAGGCCACAATCCTCCCG-3′ (SEQ ID NO 31); and set-C: 5′-GATCCAGGATAACGGAGGCTCTTCCTGTCAAGCCTCCGTTATCCTGGATCCTTTTT-3′ (SEQ ID NO 32) and 5′-GATCAAAAAGGATCCAGGATAACGGAGGCTTGACAGGAAGAGCCTCCGTTATCCTGGATCCG-3′ (SEQ ID NO 33).

### Example 10

### Construction of a Conditionally Replicating Adenovirus that Silences a p53 Antagonist and that has a Changed Tropism

A derivative of Ad5-Δ24RGD (Suzuki et al., Clin. Cancer Res. 7 (2001):120-126) expressing an shRNA directed against HPV18-E6 driven by the PolIII H1 promoter and with a modified fiber gene that expands the adenovirus tropism causing enhanced infectivity and improved oncolytic potency on many human cancer cells is made the following way. Linear full-length double-stranded Ad5-Δ24RGD DNA is isolated from Ad5-Δ24RGD viruses using a method known in the art and this DNA is recombined with KpnI/EcoRV-digested pEndK.H1_{—}18E6 (supra) in E. coli BJ5183 cells to create pAdΔ24RGD.H1_{—}18E6. Subsequently, PacI-linearized pAdΔ24RGD.H1_{—}18E6 is transfected onto 911 packaging cells and pAdΔ24RGD.H1_{—}18E6 virus is isolated and further propagated on A549 cells.

### Example 11

### Construction of the Replication Competent Adenovirus Ad5Δ24-SA-Luc that Expresses Firefly Luciferase Directed by the MLP and that is Useful for Methods to Identify an Adenovirus Inhibitory Factor and to Select shRNA Molecules Capable of Silencing Said Adenovirus Inhibitory Factor

To construct replication competent adenoviruses with a transgene directed by the endogenous adenovirus MLP, a splice acceptor sequence followed by a multiple cloning site and a polyadenylation site was inserted in place of the E3 region of a plasmid containing a partial Ad5 genome. To this end, synthetic oligonucleotides 5′-GGCAGGCGCAATCTTCGCATTTCTTTTTTCCAGGAATCTAGAGATATCGAGCTCAATAAAG-3′ (SEQ ID NO 34) and 5′-AATTCTTTATTGAGCTCGATATCTCTAGATTCCTGGAAAAAAGAAATGCGAAGATTGCGCCT GCCTGCA-3′ (SEQ ID NO 35) were allowed to anneal and were cloned in pABS.4 (Microbix Biosystems, Toronto, Canada) digested with EcoRI and PstI. The resulting plasmid pABS.4-SA-MCS contains a 32 nucleotides long sequence of adenovirus serotype 40 encompassing the long fiber gene splice acceptor site, a small multiple cloning site including restriction sites for XbaI, EcoRV and SacI, and a polyadenylation site and is flexibly designed to insert a transgene of choice. To construct replication competent adenoviruses with a transgene directed by the endogenous adenovirus MLP that can be measured by a simple and quantitative method, the firefly luciferase gene was inserted into the MCS of pABS.4-SA-MCS. To this end, the cDNA of firefly luciferase was obtained by PCR using the pSP-Luc+ vector (Promega) as template DNA and oligonucleotides 5′-GGGTCTAGAGCCACCATGGAAGACGCCAAAAAC-3′ (SEQ ID NO 36) and 5′-CCCGAGCTCCTTACACGGCGATCTTTCCGC-3′ (SEQ ID NO 37) which contain overhanging XbaI and SacI sites as primers. The PCR product was digested with XbaI and SacI and ligated into pABS.4-SA-MCS digested with the same enzymes to yield pABS.4-SA-Luc. This plasmid was digested with PacI and the fragment containing SA-Luc and kanamycin resistance gene was inserted into PacI-digested pBHG11 (Microbix biosystems). A clone with the insert in the orientation that places the SA-Luc on the adenovirus R-strand was isolated, and the kanamycin resistance gene was removed subsequently by digestion with SwaI followed by self-ligation, yielding pBHG11-SA-Luc.

To construct a control adenovirus containing the CMV promoter instead of the SA, the human CMV promoter was released from pAdTrack (He et al., Proc. Natl. Acad. Sci. USA 95 (1998):2509-2514) with NheI and BglII and subcloned in pBluescript SK(−)(−)(Stratagene) digested with SpeI and BamHI. Subsequently, this plasmid was digested with XbaI and PstI and the fragment containing the CMV promoter was ligated in pABS.4.5A.MCS digested with XbaI and PstI, thereby replacing the splice acceptor site with the CMV promoter. This plasmid (pABS.4-CMV-MCS) was used to construct pBHG11-CMV-Luc in a similar way as was used to obtain pBHG11-SA-Luc (supra).

Conditionally replicating adenoviruses expressing luciferase under regulation of the MLP or the CMV were made by homologous recombination in 293 cells between the pXC1 (Microbix Biosystems) derivative pXC1-Δ24, which carries a 24-bp deletion corresponding to amino acids 122-129 in the CR2 domain of E1A necessary for binding to the Rb protein (Fueyo et al., Oncogene 19 (2000): 2-12), with pBHG11-SA-Luc or pBHG11-CMV-Luc, respectively. This way, CRAds Ad5Δ24-SA-Luc and Ad5Δ24-CMV-Luc were generated.

### Example 12

### Expression of Luciferase by the Replication Competent Adenovirus Ad5Δ24-SA-Luc in Host Cells is Dependent on Adenovirus Replication

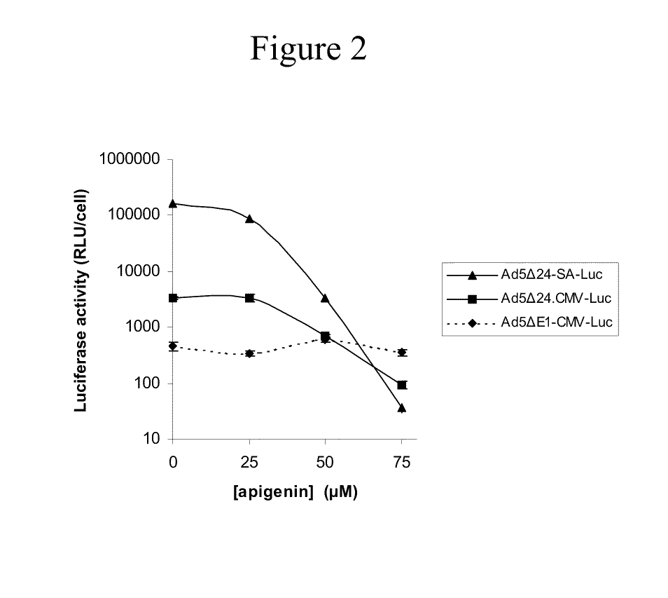

To investigate transgene expression in relation to adenovirus replication, A549 cells seeded in 96-wells plates were infected with 20 PFU/cell recombinant adenovirus expressing luciferase for 2 hours at 37° C. following which the infection medium was replaced with fresh medium with or without the cell cycle inhibitor apigenin. Because adenovirus replication requires cell cycle progression through S-phase, apigenin treatment inhibits adenovirus replication. This was confirmed by the observation that at 32 hours after infection with Ad5-Δ24E3 (supra), cells cultured in the presence of 75 micromolar apigenin contained 1,000 to 10.000-fold less Ad5-Δ24E3 viral genomes as determined by quantitative PCR. The luciferase activity was measured in adenovirus-infected cells that were cultured without or with different concentrations apigenin at 32 hours post-infection, using the luciferase chemiluminescence assay system (Promega). FIG. 2 shows the results obtained with Ad5Δ24-SA-Luc and Ad5Δ24-CMV-Luc (supra) and with replication deficient control virus Ad5-4E1-CMV-Luc. Ad5-4E1-CMV-Luc has deleted E1 and E3 regions and an expression cassette in the E1 region consisting of the CMV promoter derived from pCEP4 (Invitrogen) and the luciferase gene from pGL3-Basic (Promega) (Yamamoto et al., Mol. Ther. 3 (2001): 385-394); generously provided by Dr. M. Yamamoto, University of Alabama at Birmingham, Ala.). Luciferase expression by the replication deficient adenovirus vector Ad5-ΔE1-CMV-Luc was not affected by apigenin treatment, confirming that as expected transgene expression was not dependent on adenovirus replication. Luciferase expression by the replication competent adenovirus vector Ad5Δ24-CMV-Luc was reduced approximately 35-fold by apigenin treatment, showing that transgene expression was partially dependent on adenovirus replication. Luciferase expression by the replication competent adenovirus vector Ad5Δ24-SA-Luc was reduced approximately 4,600-fold by apigenin treatment, showing that the MLP-driven transgene expression in this virus was strongly dependent on adenovirus replication. Thus, Ad5Δ24-SA-Luc is a useful tool for use in methods according to the invention where adenovirus replication in a first type of cells is compared to adenovirus replication in a second type of cells. Luciferase activity in cells infected with Ad5Δ24-SA-Luc is directly related to replication by Ad5Δ24-SA-Luc in said cells and can be measured by simple assays (infra).

### Example 13

### Method to Identify an Adenovirus Inhibitory Factor and to Select shRNA Molecules Capable of Silencing Said Adenovirus Inhibitory Factor

Inhibitory factors of adenoviral replication or lysis as well as silencing factors capable of reducing the expression of these inhibitory factors can be identified by silencing cellular genes in host cells using RNAi, infecting these cells with a replication competent virus and measuring the effect of the silencing on replication and/or lysis. As mentioned in the background of the invention, large-scale silencing factor libraries are already available in the form of synthetic siRNAs or of plasmids encoding shRNAs. Methods are in place to perform large scale transfection of individual members of the library (Berns et al., Nature 428(2004):431-437; Paddison et al., Nature 428(2004):427-431), which can be readily combined with infection by a replication competent virus according to methods known in the art. For successful identification of inhibitory factors and silencing factors using such libraries, it is preferred to use a robust and quantitative assay for measuring virus replication and/or cell lysis, which should preferably be compatible with a robotic platform to automate the screening process. To measure adenoviral replication we developed the replication competent Ad5Δ24-SA-Luc virus that expresses the marker gene luciferase under regulation of the major late promoter (see example 12). Because expression of luciferase from the genome of this virus is dependent on viral replication, luciferase expression in cells infected with this virus can be used as a sensitive marker for viral replication. To measure lysis of cells infected by the virus, colorimetric, fluorimetric and chemiluminescent assays for the quantification of cell death and cell lysis, based on the measurement of lactate dehydrogenase activity released from the cytosol of damaged cells into the supernatant are already commercially available (Roche Applied Science; Cambrex; Promega).

### Example 14

### A Conditionally Replicating Adenovirus According to Example 8 Restores p53 Function in Cells Expressing an Adenovirus Inhibitory Factor being a p53 Antagonist and Overcomes Delayed Adenovirus Replication and/or Lysis Due to Expression of Said Adenovirus Inhibitory Factor in Host Cells

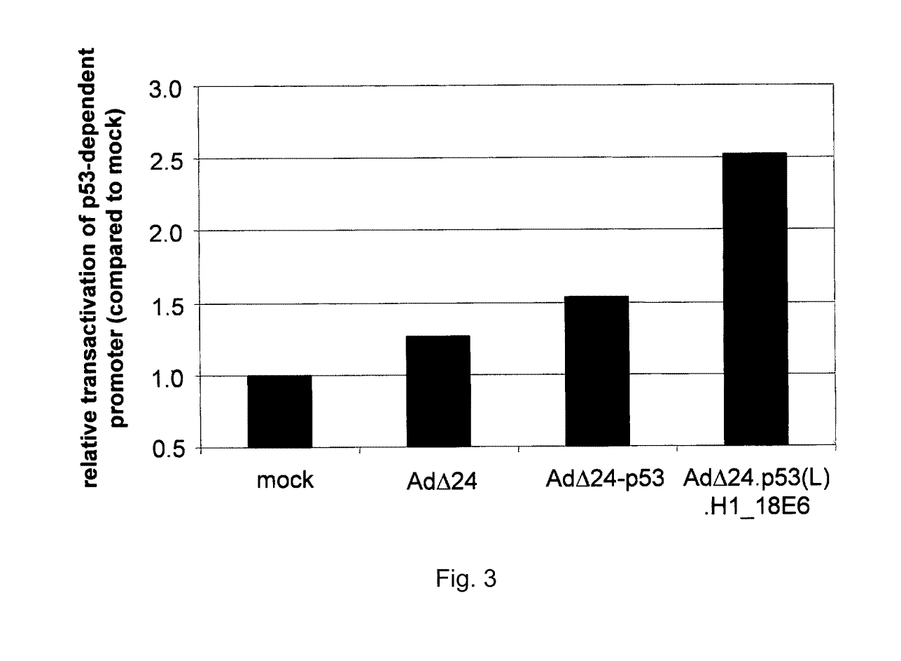

The CRAds AdΔ24 and AdΔ24-p53 (Van Beusechem et al, Cancer Res. 62 (2002):6165-6171) and AdΔ24.p53(L).H1_{—}18E6 (see example 8) were used to infect HPV-18 positive HeLa cervical cancer cells (obtained from the ATCC) at 10 PFU/cell, 24 hours after the HeLa cells had been transfected with 200 ng PG13-Luc plasmid (el-Deiry et al, Cell 75 (1993):817-825) using LipofectAMINE Plus (Invitrogen) according to the manufacturer's protocol. PG13-Luc expresses the firefly luciferase gene driven by a p53-dependent promoter. Seventy-two hours after infection, the cells were lysed in Reporter Lysis Buffer (Promega) and chemiluminescence was measured using the Luciferase Chemiluminescent Assay System (Promega) and a Lumat LB 9507 luminometer. Measured relative light units were normalized on the basis of mock-infected controls. As can be seen in FIG. 3, functional p53 expression was elevated only marginally in AdΔ24-p53 infected cells compared to AdΔ24 infected cells, showing that p53 was effectively inhibited by HPV-18 E6 protein in HeLa cells. In AdΔ24.p53(L).H1_{—}18E6 infected cells, a substantially higher level of p53 activity was measured, showing that the HPV-18E6 specific shRNA in AdΔ24.p53(L).H1_{—}18E6 had suppressed p53 inhibition in HeLa cells.

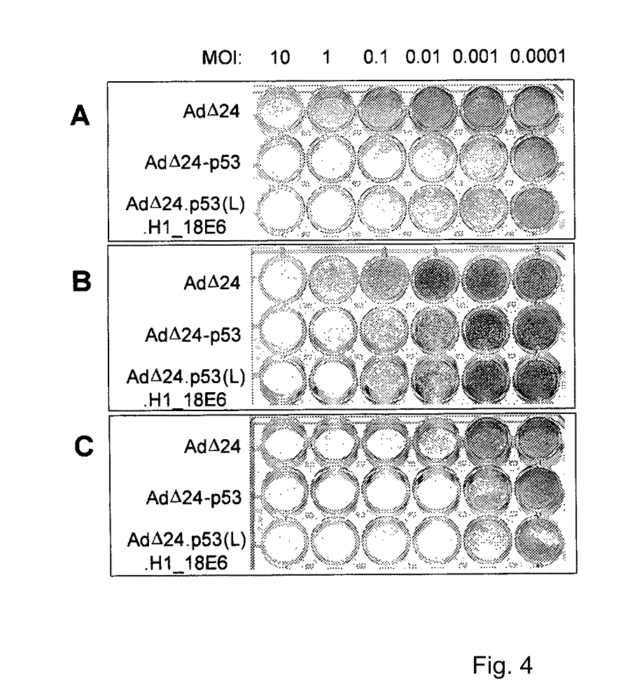

To investigate if silencing of HPV-18E6 by AdΔ24.p53(L).H1_{—}18E6 would lead to specific enhancement of adenovirus replication and/or lysis in HPV-18 positive cancer cells, AdΔ24, AdΔ24-p53 and AdΔ24.p53(L).H1_{—}18E6 were used to infect HeLa cells or HPV-16 positive SiHa cervical cancer cells (obtained from the ATCC). Since the H1_{—}18E6 shRNA is specific for HPV-18E6 and does thus not suppress HPV-16E6, SiHa cells served as negative controls. Previously, we have found that efficacy enhancement by p53 expression in some cancer cell lines exceeds 100-fold (Van Beusechem et al, Cancer Res. 62 (2002):6165-6171). One of these cell lines, breast cancer cell line MDA-MB-231, was included as positive control. Cells were seeded 5E4 per well in 24-well plates and infection was done the next day at virus doses ranging from 5 to 5E5 infectious viruses per well. After one hour, the medium was replaced and cells were subsequently cultured for 20 days with 50% medium changes every 3-4 days. During culture, the replication competent adenoviruses are allowed to lyse their host cells, releasing their progeny, which can then infect new host cells. The more effective the virus life cycle (replication, cell lysis and reinfection) proceeds the less initial virus inoculum is required to eradicate the cells in culture. After 20 days, the culture medium was removed and remaining adherent cells were fixed for 20 min at room temperature in 4% (v/v) formaldehyde in PBS, and stained using 10 g/l crystal violet dye in 70% (v/v) ethanol for 20 min at room temperature. After several washes with water, the culture plates were air dried and scanned on a Bio-Rad GS-690 imaging densitometer. FIG. 4 shows that AdΔ24-p53 was approximately 1000-times, 10-times and less than 10-times more effective against MDA-MB-231, SiHa and HeLa cells, respectively, than AdΔ24. Thus, p53 expression augmented adenovirus lytic replication in HPV-positive cervical cancer cells, but not by far as profound as in HPV-negative cancer cells. Importantly, AdΔ24.p53(L).H1_{—}18E6 was approximately 10-times more effective against HPV18-positive HeLa cells than AdΔ24-p53, but similarly effective as AdΔ24-p53 against HPV-negative MDA-MB-231 and HPV18-negative SiHa cells. This showed that expressing a short hairpin RNA silencing factor directed against an adenovirus inhibitory factor (HPV18E6) specifically relieved delayed adenovirus replication and/or lysis in host cells expressing said adenovirus inhibitory factor.

### Example 15

### Synoviolin Silencing Induces p53 Activity in A549 Lung Cancer Cells

We screened the A549 non small cell lung cancer (NSCLC) cell line obtained from the American Type Culture Collection (Manassas, Va.) and the U2OS osteosarcoma cell line obtained from Dr. Lens (NKI, Amsterdam) for functional expression of eighteen putative p53 inhibitors using siRNA. Both cell lines carry wild type p53 genes.

To perform the screens, we first constructed two independent A549 derivative cell lines and one U2OS derivative cell line stably expressing the plasmid PG13-Luc (El-Deiry et al., Cell 75 (1993):817-825). PG13-Luc carries the firefly luciferase gene driven by a promoter consisting of 13 p53-binding elements from the ribosomal gene cluster upstream of a polyoma virus minimal promoter sequence. Cells were transfected with PG13-Luc using Lipofectamine PLUS (Life Technologies), according to the manufacturer's protocol. After 48 h of culture at 37° C., hygromycin (Roche) was added to the cells to select for cells stably carrying PG13-Luc. Single cell clones were established after serial dilution of the transfected population and tested for luciferase expression using the Luciferase Chemiluminescent Assay System (Promega), as described below. Two A549 derivative clones and one U2OS derivative clone were selected for use. Cells were routinely grown at 37° C. and 5% CO2 in a humidified incubator in Dulbecco's modified Eagle's medium (DMEM) supplemented with 10% fetal bovine serum and antibiotics (100 U/ml penicillin and 100 μg/ml streptomycin).

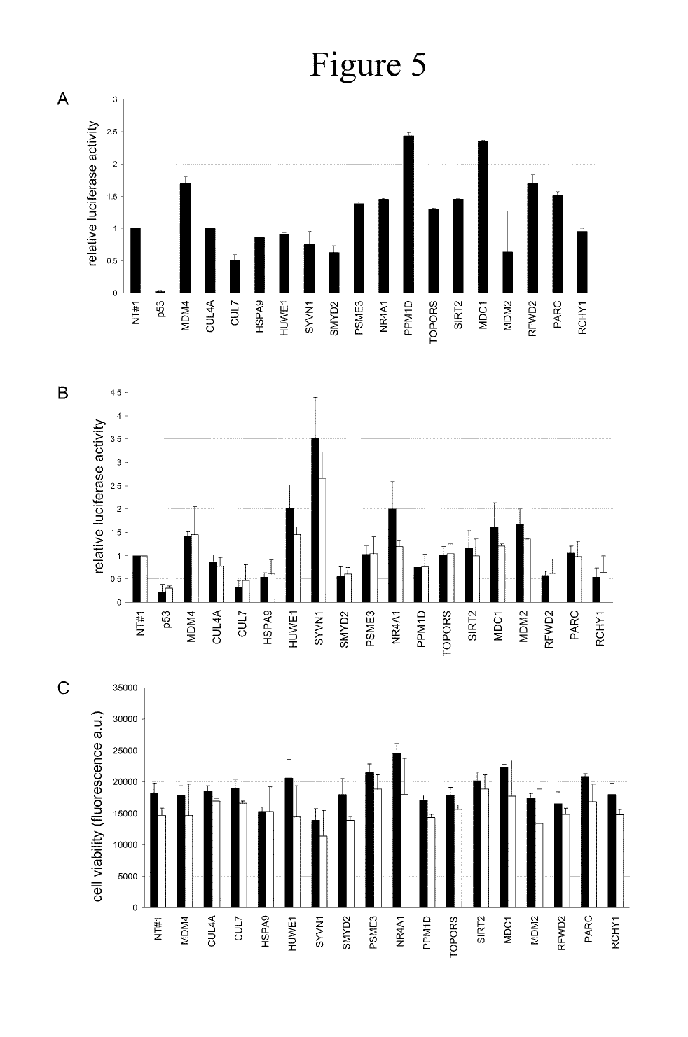

Next, p53 transcriptional activity was determined upon silencing of putative p53 inhibitors (and p53 itself as a control for the opposite effect) by RNAi. The two individual A549-PG13-Luc clones and the U2OS-PG13-Luc clone were transfected with SMARTpool siRNA duplexes from Dharmacon according to the manufacturer's protocol using 50 nM siRNA and 1000-times diluted Dharmafect 1 (Dharmacon), in 96-well plates. The SMARTpool siRNA reagents used were directed against the following human target genes (accession number; SMARTpool catalog number): irrelevant non-targeting control siCONTROL-1 (not applicable; D-001210-01), MDM4 (NM_{—}002393; M-006536-03), CUL4A (NM_{—}001008895, NM_{—}003589; M-012610-01), CUL7 (NM_{—}014780; M-017673-00), HSPA9 (NM_{—}004134; M-004750-03), HUWE1 (NM_{—}031407; M-007185-01), SYVN1 (NM_{—}032431, NM_{—}172230; M-007090-01), SMYD2 (NM_{—}020197, XM_{—}001127274; M-020291-00), PSME3 (NM_{—}005789, NM_{—}176863; M-012133-00), NR4A1 (NM_{—}002135, NM_{—}173157; M-003426-03), PPM1D (NM_{—}003620; M-004554-00), TOPORS (NM_{—}005802; M-020048-00), SIRT2 (NM_{—}012237, NM_{—}030593; M-004826-02), MDC1 (NM_{—}014641; M-003506-04), MDM2 (NM_{—}002392, NM_{—}006878, NM_{—}006879, NM_{—}006880, NM_{—}006881, NM_{—}006882; M-003279-02), COP (NM_{—}001001740, NM_{—}022457; M-007049-00), PARC (NM_{—}015089; M-014128-00), PIRH2 (NM_{—}015436; M-006966-00) and TP53 (NM_{—}000546; M-003329-01). After 72 h, the culture medium was replaced by Luciferase Chemiluminescent Assay System Reporter Lysis Buffer (Promega), and the culture plates were subjected to a single freeze/thaw cycle. Chemiluminescence was measured with a Lumat LB 9507 luminometer during the 10 s immediately after addition of the cell lysate to the Luciferase Assay Reagent. Luminescence values were used to calculate the fold induction over negative control siRNA and are given as the mean+/−standard deviation of 3 individual experiments each performed in triplicate. In parallel, silencing efficiency and toxicity of the transfection procedure were determined using the KDalert Assay (Applied Biosystems/Ambion, Austin, USA) according to the manufacturer's protocol, 96 h after transfection with SMARTpool siRNA against human GAPD or irrelevant negative control siCONTROL-1. Silencing efficiencies typically achieved 75% or higher with toxicity being less then 10%. FIGS. 5A and 5B show that whereas silencing of two putative p53 inhibitors (i.e., PM1D and MDC1) induced p53 activity in U2OS cells by more than 2-fold (FIG. 5A), considerable and reproducible p53 activation was observed in A549 cells only upon silencing SYVN1 (FIG. 5B). Silencing of SYVN1 in A549 cells resulted in 3.5 and 2.5-fold increases in p53 activity over siRNA control levels in clone 1 and 2, respectively. Silencing of SYVN1 did not increase p53 activity in U2OS cells (FIG. 5A). These observations show that PM1D and MDC1 are genuine p53 antagonists in U2OS cells and SYVN1 is a genuine p53 antagonist in A549 cells, which means that these molecules inhibit p53 function in these cells. In addition, these observations show that these genuine p53 antagonists can be suppressed in a sufficient manner to increase p53 activity in said cells by means of RNAi.

Effective SYVN1 silencing in this experiment was confirmed by quantitative real time RT-PCR (qRT-PCR). To this end, cells were collected by centrifugation 96 h after siRNA transfection. RNA was prepared by using the RNeasy kit (Qiagen) according to the manufacturer's protocol. Total RNA (1 μg) was reversely transcribed using the SuperScript III reverse transcriptase (Invitrogen) after priming with random hexamers (Applied Biosystem). Real-time quantitative PCR was carried out on a Roche LS500 instrument in a 20 μl reaction containing 10 μl of SYBR Green PCR mix (Roche), diluted cDNA and primers. QuantiTect Primers for human synoviolin (QT01669983 and QT01669983) were purchased from Qiagen; primers for internal control GAPD (5′-GTCGGAGTCAACGGATT-3′ (SEQ ID NO 55) and 5′-AAGCTTCCCGTTCTCAG-3′ (SEQ ID NO 56)) were from Eurogentec. Relative synoviolin mRNA levels were calculated as percentage of GAPD household gene expression.

This example demonstrates that different p53 wild type cancer cell lines exhibit different p53 inhibitor expression profiles. It furthermore shows that genuine p53 inhibitors limiting p53 activity in a cell can be identified by functional RNAi screening for p53 activity.

### Example 16

### Restoring p53 Function in A549 Cells by Synoviolin Silencing Increases Oncolysis by Replication Competent Adenovirus

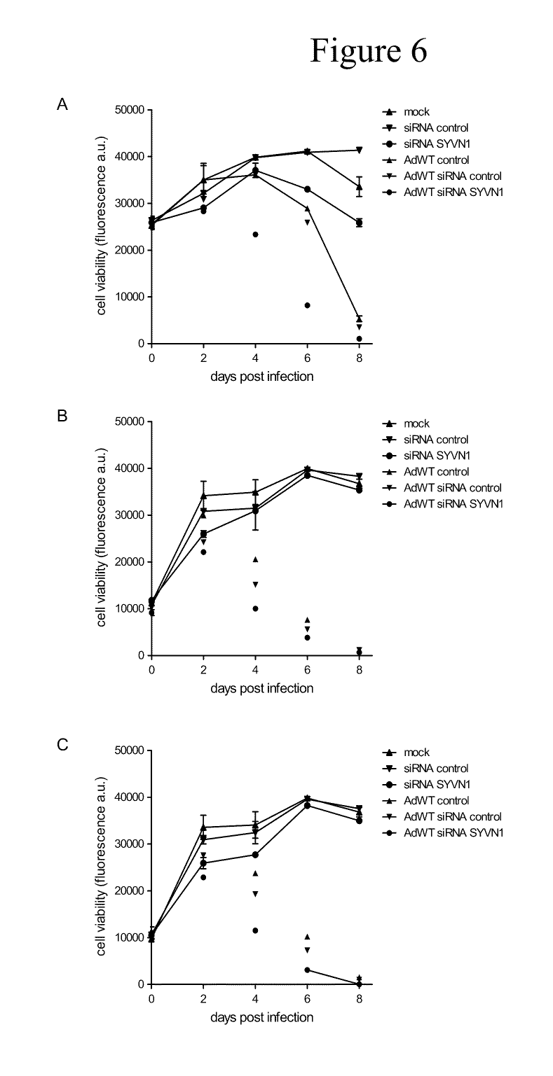

A549 cells were seeded 5×10³ per well in a 96-well plate and cultured overnight. The next day, cells were transfected with siRNAs directed against the set of eighteen putative p53 inhibitors as in Example 15 or left untreated. Twenty-four hours post transfection, cells were infected with wild type adenovirus serotype 5 (AdWT) at 100 IU/cell. Three and five days after infection, 30 μl CellTiter-Blue reagent (Promega) was added to the culture medium and plates were placed at 37° C. in a 5% CO2 incubator for two hours. After two hours, 50 μl 3% SDS was added and cell viability was determined by CellTiter-Blue conversion. Fluorescence was measured at 540 nm exitation and 590 nm emission wave lengths using a Tecan Infinite F200 reader. Cell viability was expressed as mean fluorescence (a.u.) of triplicate wells after subtraction of conversion in the absence of cells. FIG. 6C shows that cell viability of AdWT-infected control siRNA transfected cells was reduced by 50% and 60% compared to uninfected cells, 72 and 120 h hours after infection, respectively. Of all putative p53 inhibitors tested, only silencing of SYVN1 reduced cell viability of AdWT-infected A549 cells in comparison to irrelevant control siRNA/AdWT-treated cells, although only modestly. Thus, successful activation of p53 activity upon p53 inhibitor silencing was associated with increased AdWT-induced cell kill. Functional screening for putative p53 inhibitors identified a target to improve oncolytic adenovirus potency. These initial findings with SYVN1 siRNA were confirmed in several independent experiments in which cell viability was determined in two different ways.

First, SYVN1 siRNA treated, control siRNA treated and untreated A549 cells were infected with AdWT or mock treated; and cell viability was measured by CellTiter-Blue conversion assay on the day of infection and at 2, 4, 6 and 8 days post infection, using the methods described above. As shown in FIG. 6, synoviolin silencing per se had no effect on A549 cells in two experiments and decreased A549 cell viability by approximately 20% to 40%, 6 and 8 days post infection, compared to untreated or control siRNA transfected cells, in a third experiment. In contrast, synoviolin silencing decreased cell viability of AdWT-infected cells reproducibly, as compared to either treatment alone or adenovirus infection combined with irrelevant control siRNA treatment. The time required to reduce cell viability through AdWT replication by 50% was approximately 1.5 day accelerated for synoviolin siRNA treated cells compared to control siRNA treated cells and 2 days compared to untreated cells.

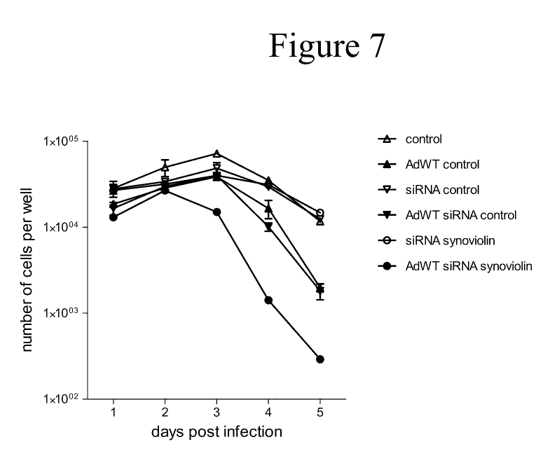

Second, 5×10³ cells were seeded per well in a 96-well plate and cultured overnight. The next day, cells were transfected with control siRNA, SYVN1 siRNA or untreated, as above. Twenty-four hours post transfection, cells were infected with AdWT at 100 IU/cell. On day 1 to 5 after infection, cells were harvested by trypsinization. Cell suspensions were mixed 1:1 with 0.4% trypan blue solution (Sigma-Aldrich) and viable cells were counted using a cell counting chamber (Bürker hemacytometer). FIG. 7 shows the mean number of viable cells counted per well from 2 independent experiments each performed in triplicate. AdWT decreased A549 cell viability on days 3, 4 and 5 after infection. Control siRNA or SYVN1 siRNA treatment alone did not affect viable cell numbers compared to untreated A549 cells. In contrast, silencing synoviolin, but not control silencing, reproducibly and selectively increased adenovirus killing potency. On day 3, AdWT caused approximately 45% cell death in control siRNA transfected A549 cells and approximately 80% cell death in SYVN1 siRNA transfected A549 cells. On days 4 and 5, these values had increased to 70% versus 95%; and 85% versus 98%, respectively (all differences significant; p<0.05).

In the aggregate, several independent experiments using two different cell viability assays showed that SYVN1 silencing increased adenovirus-induced A549 cell death.

### Example 17

### Effect of Synoviolin Silencing on Adenovirus Progeny Production in A549 Cancer Cells

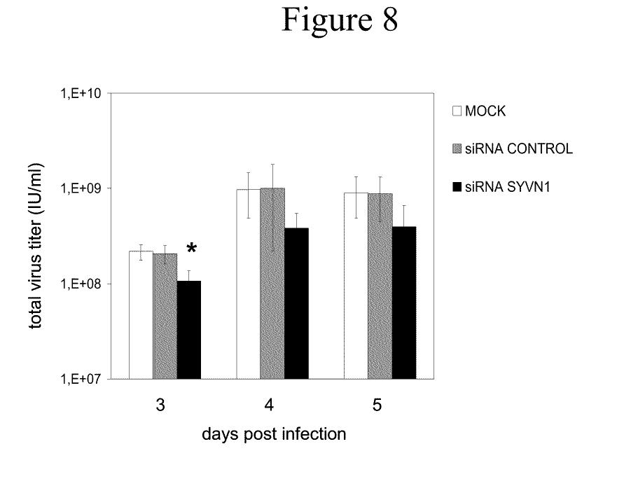

Enhanced kill of adenovirus-infected cancer cells due to SYVN1 silencing could perhaps interfere with virus propagation, if cell death is induced before the production of infectious progeny virus is completed. To investigate this, A549 cells were seeded 5×10³ per well in a 96-well plate and cultured overnight. The next day, cells were transfected with control siRNA, SYVN1 siRNA or untreated as above. Twenty-four hours post transfection, cells were infected with AdWT at 100 IU/cell. After 3 hours, input virus was washed away. Cells and supernatant was harvested 3, 4 and 5 days after infection. Virus was released by three freeze/thaw cycles. The number of infectious virus particles produced in the cells was determined by limiting dilution infection on A549 cells in triplicate. After 48 hours, cells were fixed in methanol and stained for hexon expression using the mouse anti-hexon antibody and rat anti-mouse immunoglobulin from the AdenoX rapid titer kit (BD Biosciences). The number of hexon positive cells per well was counted and virus titers were calculated. FIG. 8 presents the mean total virus titers (IU/cell)+/−standard deviation determined in 3 independent experiments. As can be seen, virus progeny production was completed after 4 days under all three conditions. The total yield of adenovirus progeny was approximately 45% reduced after SYVN1 silencing compared to untreated and control siRNA treated cells. This reduction was only significant on day 3 (p<0.05). In conclusion, induction of cell death by the combined action of AdWT and SYVN1 silencing induced by siRNA transfection one day before AdWT infection did not considerably affect infectious adenovirus progeny production.

### Example 18

### Silencing of Synoviolin Using shRNA Expression Vectors Enhances AdWT Oncolysis

We next explored whether synoviolin expression could be effectively silenced by short hairpin RNA (shRNA) directed against SYVN1. This was done using three different human GIPZ lentiviral shRNAmir expression vectors (Thermo scientific, Open Biosystems). shRNAmir constructs are designed to mimic a natural pri-microRNA transcript. GIPZ vectors express a puromycin resistance gene in addition to the shRNAmir insert. The three vectors each express a shRNAmir directed against a different target sequence on the human SYVN1 mRNA. The sequences of the three shRNAmirs are given as SEQ ID NOs 38-40. A549 cells were transfected with the three vectors and with a GIPZ lentiviral empty vector control (Thermo scientific, Open Biosystems). Stable transfected cells were selected on the basis of puromycin expression. Silencing efficiencies were determined by qRT-PCR as described in Example 15. All three shRNAs silenced SYVN1 expression at least 50%. Maximum silencing was obtained in A549 cells stable expressing the shRNAmir with SEQ ID NO 40, which silenced SYVN1 expression 84%. In addition, we determined p53 mRNA expression by qRT-PCR. This was done using the same qRT-PCR method, but with synoviolin-specific primers replaced by TP53-specific primers QT00060235 and QT00060235 purchased from Qiagen. No significant differences were observed in p53 mRNA level between synoviolin shRNA and empty vector control shRNA expressing A549 cells.

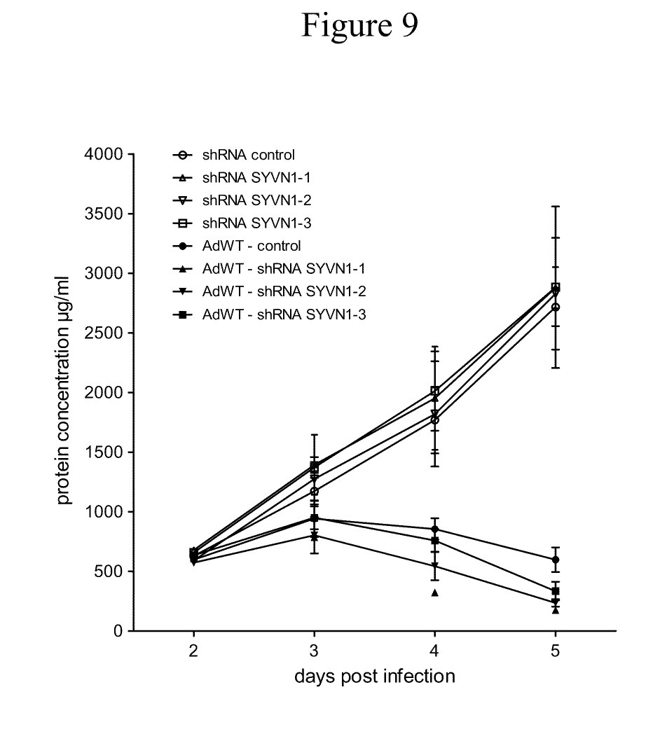

Stable lentiviral shRNAmir transfected A549 cells were seeded 5×10³ per well in a 96-well plate and cultured overnight. The next day, cells were infected with AdWT at 100 IU/cell. On days 2 to 5 after infection, as a measure to quantify cell viability, protein concentrations were determined using the BCA protein assay (Thermo scientific, Pierce) according to the manufacturer's protocol. Briefly, culture medium was replaced by 25 μl lysis buffer after washing with PBS. Cell lysates were mixed with BCA reagent and plates were placed at 37° C. in a 5% CO2 incubator for 30 minutes. After 30 minutes, 50 μl 3% SDS was added and BCA complex formation was measured. Absorbance was measured at 595 nm using a Bio-Rad model microplate reader and quantified. Protein concentration (μg/ml) of each sample was calculated using a BSA standard curve. FIG. 9 shows the mean protein concentrations of triplicate wells of 3 independent replicate experiments. As can be seen, stable synoviolin silencing alone did not affect cell viability. In contrast, it significantly reduced cell viability after AdWT infection. A549 cells stably expressing synoviolin shRNAmir with SEQ ID NO: 38 were most vulnerable to AdWT oncolysis. Hence, more effective cell killing by replication competent adenovirus is also accomplished by SYVN1 silencing using short hairpin RNAs. All three selected SYVN1 shRNAmir sequences are useful for the invention, with SEQ ID NO: 38 providing maximal adenovirus oncolysis enhancement and SEQ ID NO: 40 providing maximal target silencing, respectively.

### Example 19

### Construction of Six Different AdΔ24-shSYVN1 Oncolytic Adenoviruses Expressing a Short Hairpin RNA Directed Against SYVN1

The previous examples provide ways to construct conditionally replication competent adenoviruses with shRNA expression cassettes inserted in place of the adenovirus E3 region or between the adenovirus E4 region and the right-hand ITR, using Gateway recombination methods. Here, these teachings were followed to construct conditionally replication competent adenoviruses expressing shRNAs directed against SYVN1 inserted between the adenovirus E4 region and the right-hand ITR.

First, a shuttle vector was made carrying a Gateway recombination destination cassette between the adenovirus E4 region and the right-hand ITR. To this end, the construct pEndK/SpeI (generously provided by Dr. R. Alemany, Institut Catala d'Oncologia, Barcelona, Spain) was used. pEndK/SpeI was made by first digesting pTG3602 (Chartier et al., J. Virol, 70 (1996):4805-4810) with KpnI and religating the vector fragment comprising Ad5 map units 0-7 and 93-100 to create pEndK. Next, a unique SpeI site was introduced into pEndK by changing Ad5 nucleotide 35813 from A to T by site directed mutagenesis to create pEndK/SpeI. PEndK/SpeI carries PacI restriction sites flanking the two Ad5 ITRs. pEndK/SpeI was made compatible with the Gateway system by ligating the Gateway destination cassette rfa (Gateway Vector Conversion System; Invitrogen, Carlsbad, Calif.) as a blunt fragment into the SpeI site (filled in with Klenow polymerase). A plasmid was selected that contained the Gateway destination cassette with the coding sequence of the ccdB gene on the adenovirus R strand and was designated pEndK/DEST-R.

Second, Ad5-6.24E3 (Suzuki et al., Clin. Cancer Res. 8 (2002):3348-3359) linear dsDNA was isolated from virions and recombined with linearized pEndK/SpeI (supra) in BJ5183 bacteria to obtain plasmid clone pAdΔ24E3, from which full length AdΔ24E3 DNA was released by PacI digestion. This DNA was recombined in BJ5183 bacteria with KpnI-digested pEndK/DEST-R to obtain pAdΔ24E3-DEST-R. This plasmid carries a full length adenovirus genome flanked with PacI sites, comprising the E1AΔ24-mutation (Fueyo et al., Oncogene 19 (2000):2-12) providing conditional replication property and the Gateway destination cassette between the adenovirus E4 region and the right-hand ITR. pAdΔ24E3-DEST-R is propagated in the E. coli STBL2-DB3.1 strain, which contains a gyrase mutation that renders it resistant to the lethal effects of the CcdB protein thereby allowing propagation of plasmids carrying the ccdB gene in the DEST cassette.

Third, SYVN1 shRNA-encoding sequences are introduced into entry clone pSHAG-1 (Paddison et al., Genes Dev. 16 (2002) 948-958; generously provided by Dr. G. J. Hannon, Cold Spring Harbor Laboratory, NY). pSHAG-1 contains a U6 promoter-driven expression cassette flanked by the Gateway attL1 and attL2 recombination sites such that the expression cassette can be transported into destination plasmid vectors including pEndK/DEST-R and pAdΔ24E3-DEST-R using the Gateway system. shRNA-encoding sequences are introduced by ligation of pSHAG-1 digested with BseRI and BamHI with two annealed synthetic oligonucleotides with compatible overhanging DNA sequences. The first of the two oligonucleotides contains in the 5′ to 3′ order: a first stretch of nucleotides complementary to the SYVN1 mRNA (i.e., antisense), a loop sequence, a second stretch of nucleotides of the same length and of reverse complementary sequence to the first stretch of nucleotides, and a stretch of at least 4 thymidines. The second oligonucleotide is reverse complementary to the first oligonucleotide. Furthermore, when annealed the then double-stranded oligonucleotides form overhanging sites compatible with BseRI and BamHI restriction sites. Sequences of 6 shRNAs directed against SYVN1 and of 6 sets of two synthetic oligonucleotides useful to insert these shRNAs into pSHAG-1 are given in the sequence listing. The shRNAs with SEQ ID NOs 41, 42 and 43 comprise the same stem-loop sequence as the shRNAmirs with SEQ ID NOs: 38, 39 and 40, respectively; but lack the pri-miRNA flanking sequences. The shRNAs with SEQ ID NOs: 44, 45 and 46 contain the same target recognition sequences as the shRNAmirs and shRNAs with SEQ ID NOs 38 and 41, 39 and 42 and 40 and 43, respectively, but contain a shorter loop sequence. The oligonucleotides used to create shRNAs with SEQ ID NO 41-46 are listed as SEQ ID NOs 41 and 47; 42 and 48; 43 and 49; 44 and 50; 45 and 51; and 46 and 52, respectively. Annealing of the three sets of complementary oligonucleotides with the long loop sequence followed by ligation into pSHAG-1 digested with BseRI and BamHI results in the formation of pSHAG-shSYVN1-1, pSHAG-shSYVN1-2 and pSHAG-shSYVN1-3. Annealing of the three sets of complementary oligonucleotides with the short loop sequence followed by ligation into pSHAG-1 digested with BseRI and BamHI results in the formation of pSHAG-shSYVN1-4, pSHAG-shSYVN1-5 and pSHAG-shSYVN1-6. pSHAG-shSYVN1-1 and pSHAG-shSYVN1-4 encode shRNAs homologous to nucleotide 648 to 669; pSHAG-shSYVN1-2 and pSHAG-shSYVN1-5 to nucleotide 741 to 762; and pSHAG-shSYVN1-3-pSHAG-shSYVN1-6 to nucleotide 2952 to 2973 of the coding sequence of the synoviolin gene, respectively. Fourth, the shRNA expression cassettes from pSHAG-shSYVN1-1 to pSHAG-shSYVN1-6 were transferred into pAdΔ24E3-DEST-R via an LR GATEWAY in vitro recombination reaction using the GATEWAY LR Clonase enzyme mix (Invitrogen) according to manufacturer's protocol, to create pAdΔ24E3-shSYVN1-1 to pAdΔ24E3-shSYVN1-6. Finally, full-length AdΔ24 CRAd genomes with inserted shSYVN1 expression cassette were released from pAdΔ24E3-shSYVN1 constructs by PacI digestion and transfected using lipofectamine reagent in 911 cells or A549 cells to obtain shRNA-expressing replication competent adenoviruses AdΔ24E3-shSYVN1-1, AdΔ24E3-shSYVN1-2, AdΔ24E3-shSYVN1-3, AdΔ24E3-shSYVN1-4, AdΔ24E3-shSYVN1-5, and AdΔ24E3-shSYVN1-6, which were further propagated on A549 cells according to standard cell culture and virology methods known in the art. The E1Δ24 deletion and the U6-shSYVN1 insertion and orientation are confirmed by PCR, shSYVN1 sequences are confirmed by sequencing and functional virus titers are determined by limiting-dilution titration according to standard techniques.

### Example 20

### Enhanced Adenovirus Induced Cell Kill Upon Insertion of Sequences Coding for shRNAs Against SYVN1 in Conditionally Replicating Adenovirus

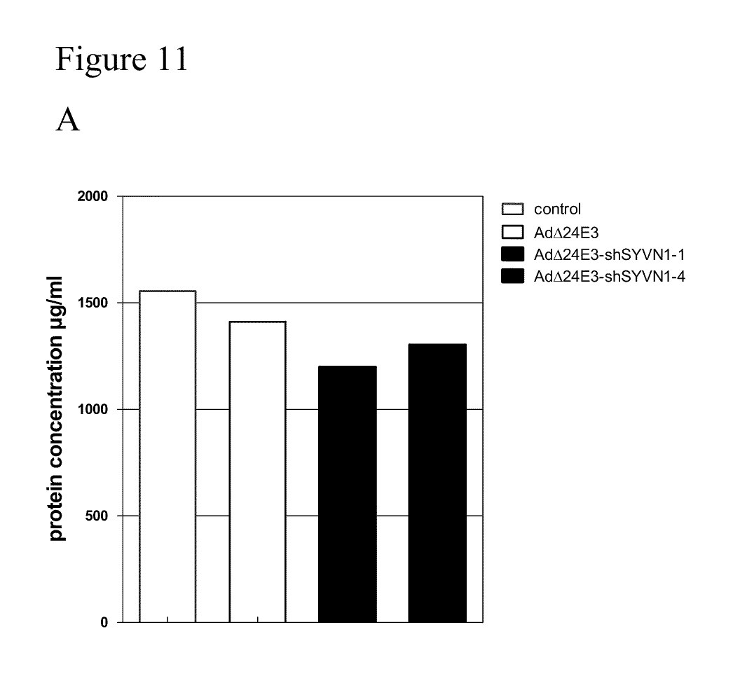

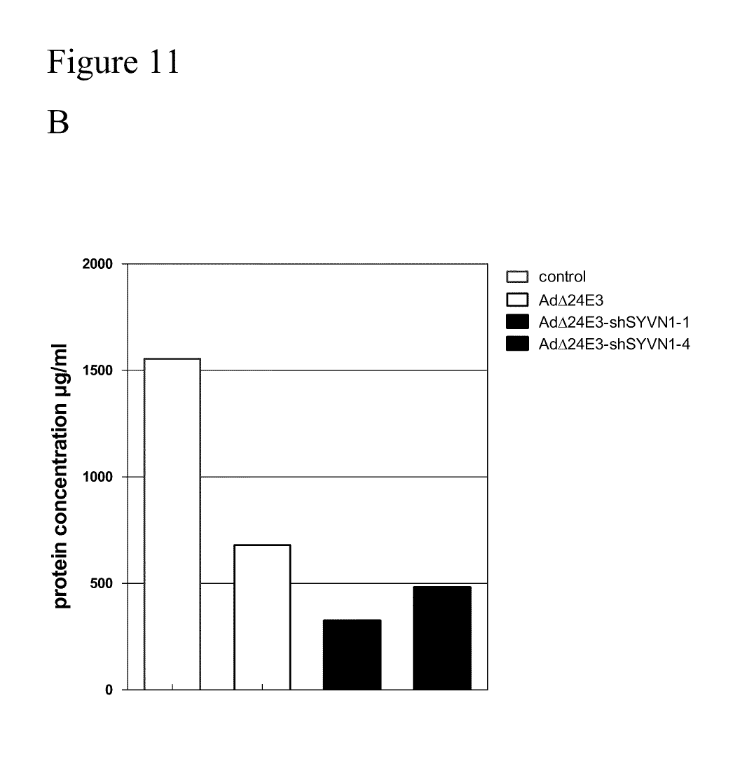

A549 cells were seeded 5×10³ per well in a 96-well plate and cultured overnight. Two days later, cells were infected with AdΔ24E3, AdΔ24E3-shSYVN1-1 or AdΔ24E3-shSYVN1-4 at 10 and 100 IU/cell. On day 3 after infection, as a measure to quantify cell viability, protein concentrations were determined using the BCA protein assay (Thermo scientific, Pierce) according to the manufacturer's protocol. Briefly, culture medium was replaced by 25 μl lysis buffer after washing with PBS. Cell lysates were mixed with BCA reagent and plates were placed at 37° C. in a 5% CO2 incubator for 30 minutes. After 30 minutes, 50 μl 3% SDS was added and BCA complex formation was measured. Absorbance was measured at 595 nm using a Bio-Rad model microplate reader and quantified. Protein concentration (μg/ml) of each sample was calculated using a BSA standard curve. FIG. 11 shows the mean protein concentrations of triplicate wells of one experiment. As can be seen, cell viability after AdΔ24E3 infection at 100 IU/cell was reduced to 44%. Cell viability after infection with Ad24ΔE3-shSYVN1-1 or Ad24ΔE3-shSYVN1-4 was reduced more effectively to 21% and 31%, respectively.

1. Replication competent adenovirus, being capable of replicating and having lytic capacity in host cells, the virus comprising in the genome thereof, at least one DNA sequence coding for a silencing factor functional in reducing expression of a target gene in the said host cells, said virus further provided with one or more expression control sequences that mediate expression of the DNA sequence in the host cell.

2. Replication competent adenovirus according to claim 1, comprising at least one DNA sequence coding for a silencing factor functional in reducing expression of synoviolin in a host cell.

3. The adenovirus according to claim 2, wherein the silencing factor comprises a short hairpin RNA.

4. The adenovirus according to claim 1, wherein the length of the double stranded region of the short hairpin RNA molecule is between 19 nucleotides and 30 nucleotides per strand.

5. The adenovirus according to claim 1, wherein the adenovirus is a human adenovirus, preferably of serotype 5.

6. The adenovirus according to claim 1, wherein the adenovirus is a conditionally replicating virus.

7. The adenovirus according to claim 6, wherein the virus is an adenovirus carrying a mutation in the ElA region encompassing at least a part of the CR2 domain of ElA, preferably a deletion encompassing amino acids 122 to 129 (LTCHEAGF) of ElA.

8. The adenovirus according to claim 1, wherein the expression control sequences comprise a U6 promoter.

9. The adenovirus according to claim 2, wherein the host cell is selected from a lung cancer cell, a breast cancer cell, a head and neck cancer cell, a liver cancer cell, a melanoma cell, a thyroid cancer cell, a colorectal cancer cell and an urothelial cancer cell.

10. The adenovirus according to claim 2 for use in a medicament for the treatment of cancer, wherein the cancer is selected from lung cancer, breast cancer, head and neck cancer, liver cancer, melanoma, thyroid cancer, colorectal cancer and urothelial cancer.

11. The adenovirus according to claim 2, wherein the at least one DNA sequence coding for a silencing factor functional in reducing expression of synoviolin comprises one or more of SEQ ID NO 38-46.

12. A kit of parts comprising at least one DNA sequence coding for a silencing factor functional in reducing expression of synoviolin in cancer cells and a replication competent virus, preferably an adenovirus.

13. The kit of parts of claim 12, for use in a medicament for the treatment of cancer, wherein the cancer is selected from lung cancer, breast cancer, head and neck cancer, liver cancer, melanoma, thyroid cancer, colorectal cancer and urothelial cancer.

14. A method of lysing a cancer cell comprising the step of providing the cancer cell with a virus according to claim 1 thereby inducing lysis of the cancer cell and release of virus progeny from the cancer cell.

15. A method of lysing a cancer cell comprising the step of providing the cancer cell with the kit of parts of claim 12, thereby inducing lysis of the cancer cell and release of virus progeny from the cancer cell.

16. Method for treatment of a subject suffering from a cancer, whereby the cancer is selected from lung cancer, breast cancer, head and neck cancer, liver cancer, melanoma, thyroid cancer, colorectal cancer and urothelial cancer, the method comprising the step of administering to the said subject an effective amount of the replication competent virus according to claim 1.

17. Method for treatment of a subject suffering from a cancer, whereby the cancer is selected from lung cancer, breast cancer, head and neck cancer, liver cancer, melanoma, thyroid cancer, colorectal cancer and urothelial cancer, the method comprising the step of administering to the subject an effective amount of the kit of parts according to claim 12.

18. Replication competent virus according to claim 1, wherein the virus genome further comprises the coding sequence of at least one restoring factor functional in restoring the p53 dependent apoptosis pathway in the said host cells, operably linked to one or more expression control sequences, functional in the said host cells.

19. Method according to claim 14, wherein said host cells are present in an animal body, preferably a human body.

20. Method according to claim 15, wherein said host cells are present in an animal body, preferably a human body.
